SEQUENCE LISTING (109 sequences)

SEQ ID NO: 1 - DNA, 1181 bp, Zea mays
cggtgagggattgacgcataacagaaataactagtacctttctataaggcaaaattgagcacagtgattcactgattgtgtcccctagcttctctttccccacctacaggttgcaacgcaaaagcaagcaggaaaaaaagaaacacatttagcagtctagactctaacatcatccaacgattcatggcctgttggcatgttgctggaccgtcactggttcacaggcctttagaggatacttggccagagcgggcgcgcgtgcgcggcggcggagtgctgtggtatccagtgcgtgtgcgccgatgtggaggccaacgcagcgggcctgggctttgaggccgggatggggagcctcagggagtgcggcgacggctgcgcatctgcgccgtcatgtgggaaccgacggactcagcgcggcgtcgctgtgcggctgtgcgtcttgcgccacctgcacaaggtggtttgtgtgcgagtacgccggtaaagcagggcatatgcctctctctctggattctttgcgcttgcatactaattgttggtttgcattttgagctgaaccaatctattaaaatcgctggacgactattaaaatcgctggacgacgtagttctgtacttctgttccatgacggacgaaatgattccggatgaagctgtgcgcccgtgctgcattccaaactgcaaaaattgctgattgctggacgcggctccgtccgacggcgtacaaactgattctatatggaccgatgaggcgatccaaacagactactaaactcctacttaccaagcaaaacaattgatcaatgattacccgagtgccaaacgggcactatgaacacagaacccataacataataagctaaactgtactaatgcacaagatgcattaaaaaatgccgttttagtagattgttaaccatgtttgtgctctccacatgtcatagatatattcttataatttgttcacctgtgtttccggttagttgttcatctatgcctagctttgctttggttctgttcttgtcatagctgaaaaaagaagcgaattcattgggcaaagccttatatatagttgatacataaggattcatgtaaaatacatttgcagtccggtacagtttttactttttagccttgtgccctttgcaaaaaggactacaagaatcatgccttttttcagcg

SEQ ID NO: 2 - DNA, 1499 bp, Zea mays
ttggtttggatatttgaaaattggaaattagaaattcaaatagaaaaggaatttagaaaagaaatggggggaaattcgtgtatttgggtcgtaattgtccagacagcccaaatccaccgcgctgccctgggtgtgggtctctgaccggtggctcccgctcctgctcactggcctccccttcccgcgtgtggttgttgcgctcactggcacgcgggcctactgctcagtctccagtcgcgctcaggaatctattctcacggacgcgtgggccaatgtggccaaccagaacacttccccatcgctcgcgtggcccatccctttacgctttcgcgcctgctgacatcgcggcccaatcaccagcctcctcaccctcacatgcgctggagaaccctggtcgcagttaaatgggaccacactctcaggtcgccttccccaactcgccgcagatcttgcacggtaacaccatgcttgaccgatgtctacctgccccgcttgcttcgatcgtggctgttacgccctcgcctctgggctataagaccccaagcctcgcgccgctcacctcccaaacaccatggccgtggatctcgctctccggtgaacggcgcccacccttcgtcctcctccctctggccctgtgactagcggacgggccattggttctcgctaaggtcgttggtgtcggcgtcctcgcgattgatgctctccagcagcgcacccgagtcctgtcgccgctgcccttgcttggcctcgtcgcatcctcgacccaggcgcgcccaccgtcgagctttggggagggaatctgtgcgcatacgccggtgtacgcctgcgccggttcccaacccttggaagaccacctgtgatgctccattgcggtgctcggatggtgctcgcggcatcgcactgagtcgtccgtgtcttgggagcacgtgatttctcgctatggttaagtcgccgcctcggtgccgctcttctccctgggccgcgtcgcacatgatctagtttctggtaagttgttacccgctccattcgccttgcactgagcaacgtgtagcatctgtttgaactagaaatagggcgccggagcatgtagaagggggtccgtccatgggcgccgccgtgccctgttgggcggggtgggagaagcgttggtggccgttgatcccagaccgactgtggcaatcatatctcgcatacccttttgcttggatgagtattgaccgttcgatctagatgtgcgtacgagattagaaaaccgcgtaccgctttttagataaaatccgagccgtagattgtagatctgagggctaggtcgagtcgtagacatggaactccctcgaccatcaatctcgtatccaaggcacccgagagcgtaccgatacattaagttgtatggtttaatctgggccgtaggattagatatgagtggtcgtggttggcagataccccttcgccgagccttatttgcaaaagacaccccgtagaatttaataacaaacccg

SEQ ID NO: 3 - DNA, 915 bp, Zea mays
gttcaaagcttgcgtgcagatactcccgtaaaacacgcatccgctcgtgtgcagttcaatcacaagtttgtggatggttggaacgcagtcacccgtcaccccacggccccacgtggccgtgacacggtcggttcaaagtccacggtgtaccacttccacccgtccccaccccaccatccttctctacttctctcctctctctcccaacggacccgatctcgtacctcacttccgtcccgcccagcttcgcttcacggcgcgccgaccggcttggacgcgccgaccggctcgtgatcagctcggtcggatccgacggtcgctcgctgctgcagcgggcgcttgctgtgcgtcgcgtgggctcggccggagcccggagggaggcggagccttccccgggtctcgaagatggcggcgccgccggcgagggcccgggccgactacgactacctcatcaagctgctcctcatcggcgatagcggtgagtgccttcaccatcacgcctcctccccctctcgctctccgtccggatcgattggctgcggtgagatctgagtgcgaaatcgaagttcggttctgattcgtccggatcgactgtgaaacgcggatgctgctgggccctgctcctcccaaaagctgccgctgtttgcctgtttcgtcgatgacgtgcaacggttttatgtatataggaatgatatgctgaaaactcacccaccatcgggtgatgactactttgctagttgatgcttgagagctacatttacaaccacacagacaagtgaaccgaaattccatttggaaaccgggatgcgacaacggacgctaattacaaaataaacagcctttgccgtcttatctaactgttacattggaatggtgattgtcggtgctcacagaaaaggaaatgcatggattttg

SEQ ID NO: 4 - DNA, 1145 bp, Zea mays
gggcgtagcagtgagcgggacacggagcggcggcggcggcgccatcgagcgcgagcaggaacaggcaaacaggagctagcgcgagtgcgtgacctagggacctagcgcgacccagtgggcagcagcgtcttgacgtattcgtattcgatatccgaagcaagtatccgaatccatatccgatatccagaaaaaacgttcgaatatccgaaaaaaatatccaaatcattatttgctctctttggatacggatacggacgggtttacatccctagctggtgggcacgcggacgcggtggggtggggtggggtggggtggtggggtgcgttgcggtgcggctagacgggacggagccttgccgttaacttgagacggcggtttgataggatccgagccgacatggtgcgtcggatcgaaaccaggaccgaccttggtgtctcgttttccaggtcgaaacggctagcggttccttttaaaccgccgtcagcgtctctcggcgtcgtggcgaagaaaccggacgactcgtcggtctctgtgagctgtgacgcaagctactgatcaccatcacctgctggcgcggggacggcggcgacccggcctgcgacgggcacacgcccacacgcggacgcggagctgtttgctgcgcctgcgcggcaggacaccggcggtttggatgctcctgccacctgctcgccggtaggcgggcggcgcaccgcgcgcgtgtcccaaagtcagccgcaaaatgaagagaaacacggaggacgcaacacgtcgtcgacgcgaaggctgatctcgctagcacggagcacgcccatcatcatccgcctcgatctgctaacccgcgccatggccccatttcgcccgtccgtccaatgggatggatctttttttttttttgaacggaccccggacccggagggagtcgatcggaacgaggtacagtaacaggaccacatatgtgtggtctcgcagttccgataagaccagacaccggcccatctcggaccggacgtgctcctctacgaactatgggcggattcccgctttccggaggaagtaaatccctccgttaaaaaaaaactgtgcttgtattcctttgatcacaggagtatggacccgcttgtttcaactttagctttttcaaccg

SEQ ID NO: 5 - DNA, 1163 bp, Zea mays
ggtgcagcagaagcaacaagccccaaagtggatagcggccaaagcaaccagcggaagcttcttaattacggctaggcttcttaattaagtgcccatcaaatcaagctgccaacgcaggcagccagccactctcgagaagcgtatccggggcccacaaggtggaatcaacgttcaccctgccgtgacgccttgtccccagctaccccgccgtctgcctaccccattcaccgcacctccatggcccacgcgtcagtggtcctgtgactgggtgggcaggtgagcgtgagtccgtgagaaggaagcaagccctgttccgccgttcgctcgtcccatccgataactttaccgtgtgtactgtgattgtcgctctgacccgcggcacccgtgctgaagcctcccacgaccctcgtggcacggtgggacatagcgacaggcgcctcacgctcctcccccctcccctctcttcccccctgccaggcaagagagaagaaacgcacgcgagccgcgagcagaatacagcagcacgggcgccagcgccatcctgatctcgctatctcccttctcttctcccccgatgcgatctcgccaaaccctaatcccgccgcccgccgcgcggccgtcgccggagccctagccccgaatgtggcggtgacgcccgtgctgctgtattctgctcgcactggtcctgctcgtggagcacggcatcggtgagcggggccagcgccgcgggcggtgcgcgcacggagaagtccgccggaggctgccgaggcagcgtcaatgagggagatgagcgcgggcttggccttgaacgccgcgacgcggaggagcttgtagaggcacgggaggagcccccggaggtacacggcgaggtcgtcggtgggctccgaggcctcgacggcggcggcggcagcgagcgcggcggccagctgcgcgcacttgtcctgctcgtggagcacgacgtcggtgagcggggccagcgccgcgggcggtgcgcgcacggaggagggacgcagcgcagcgcggcggctaggagcctggtcacgagcggcgcgacggcatcccgcgggtgctccccagcgacgagcgccagcagccgaagcgagtgatgtcggatgcggaggtggttggaagcgcggcggggtttgcgggaggacgtgcgggca

SEQ ID NO: 6 - DNA, 4230 bp, Zea mays
tgtactgacggtgtctgactaaatcaccaaaaaatcgtttggcactcgtcgaattgctcctgctcatggttaggagggggcagagtagctaccgttgctcgctctggttagctttcgctgaggtggtcagcagtcagcacagtggagttctctcaggcaattacgcccctgttgtacagttcctttttttctctcttcggttccgtggaagttgttagcaaattttctgcagtatgtttctcttcgacgcttatacggtccgctctgtaaaagctgggcagctgcactgaaggtctgaagcagcatacaaccttgcggctctcaaaacatgtttgctccattttacagcttctaacaaactacaattcccaacttacagcttgcaacttaggagggaagacggtttcactggacaacaatgagagaaagcaagagcaccgaatgggaatgtcacacacaaacgcacacgtgacgcaccccggcgacgatcaacatggatgaccccaggcccggcaacagcattaacgcgtcacattgcgccggccgaaagaaccgatggatgattccttgcgttcgcagcaaacccttgacacggcggcgagcgaccacctcggcccggcccggccccgtaccaccacgtcggtgcctacacctgcgcctgcgcctgcgcgtctgcactgtccgagacggggacgaatgggcctggtccagccggtggaccacctgatcataaccaactcaaaaggcatggtccctacgcacccgtgtttttcccccctccaccctttttttttcccacggacgcgacgggtgccgtgccgtgccgcgacgccacgtacgtccggtccacgccgaacccagctcccgctgtctgttgctggctggtgcgacgcgacgccgaaatatcctggcctggccccgacccgacgcatcgcgtgcgtcgtgctcgcgcacaactcaccccccatcccgcgccccatgcgcactgcagctgtggtggccggacgcgaggggcgcacggcagggcacgagacgaccgcccacggggggaatctgttccatgcatgcaccagtaggagaaacagaaactgtttcgacgtctctgcaagcaggagcgcgatgctgttgacgccgttggaaagacgacgcgcgcgacacggcaacacaacagaacatactcccgtagctagcgcacgtagtagtagtaggccgctgcaagattcgatctttcatcgtgctggttgcacgctatcgttgttcgctagtacgtattcctactacatacataatagctagccagcgtcgcctagggcgatgcaacacctagtaggtgcctaaaagcgtaggcgcggacgcgcgcgtcgagtcgactcgactcggaccttccacttccaaccaactaaaactctgcgcggttgcggtgcgcccccggcgggcatcaaagagcctccttccaaacccatgcccgagcccgacgacgacagccacccgagactcagaagcacggcacatacgatcgtgacaagtgatggagaggcagacacgaaatctgcttgccgaaaagcctactagcggtggcagataatgggaaacctaacaccactctcgaaaacatcagaaggaaattacaacaaaaaaaggagctttcgctcccgctccagcgatacacgccgtgatctcgaccagagcgcccccccttgccccagtttgacaacagctgcctgctgctgctagcaactcgtgccggcctgttcgtttcgcaacagtgaaagcgtggatggccggtgcaaatgcaatgcaactcatcaccgatcgccacaaaaagtccaggcgcttttggctccctgctcttagtgctctagtgccccctcctgcaaggctgcaatttgcgattcgcgagcagtgaccgtgaccgtgaccgtgaccgtgaaaacgcgcgagctccccggccaccgcactgcacggagcacacacacaactagcccgcgcctttgccaacagctcgaaacccttgcgggcttgccgcggccgcgcgcgcgttttcacctctgatctcgtgcgcggcgcggccgggtcggaccggaacgaaagggccaaaggggtagcgacgctacgtcgctactccgccgtacgtccgcgccaccgcctgcgcaggaaggaaggaagggaacccggccgtccgtccggccgggcactagggtggtgcctgcctgtggcgttgccgcttcgggttggctcgtgctccgtgcttgcaattccagtgccgtactgccgtccatggatggacggacgtcggcagaaaaaggcgaagatggaccagagcggaaaaggagccgagctccagcagcgagcagcgtcgtccgtcccccacaggccacgccaaaggcaaccctgctggctcgcatcgctttcctagccgccctaggcgggggggtcgcctgtgtctttcgggcaggtgctgtgccttggcttgtcgccccattccctcaccgccagcgcccagcagccagcacctcgtttgggttttggatgcagcactgcacgattccctgacagctcggaaagggtacagagcctgcggttggggataagaacagtggtgagccaaaaaggttgggtagctcgttcaaccagcggaaccagtattttttatattagttcatattgttagttaacgagcctatcaagtcaaagctgtcgagcttaaatttatattaatctcatattgtactatgtctcggttgattgttagacctagtcctataaaagaacattatcatgacaatcaatctattttctttaaactggtaagaagataactatggagccctagatagactagtccgtcgttattcctccctccttcctccgggacaggtagctcgcgtcgtgggggcagcgcaagcacgcggtcccggtctttgaatatgtccggggatttcagcggacgcatgtggcgtccagtacgtgagcagggtactatgtttcactttcaagggatgagcaactttcagtttaaccaagccttattaccgaagtagacagaagtaaaacacaagttaccgaaggctgtttccaccactcgaaaagactccgtggcacgcagaggaggacagaattctcaaatcagtacgcgctgcctgctgccctgtgctgtgcttcatcggagcaagagcatgtgctgggctggaatctcgtcccgtaacgattcggttcctcgtcatgttctgggatttttgttcatccttttattttatttatttttttaccgcgacgcgagatcacctttggtcccccctcccccggtcctctcctcctgctggctgccatctgccaagccaagggcgctgccgctactgtgcctgaacgctgccgcgccacagcggcagcctcgcagtgagatcccaggcacccattcaattctgcagttctcgctgccgcccttccctctctgtcttctccgcacggcgaacagtgcgttctcgtccgctgaggccgaggctgtgctgatcgagtttggtcgcttgtgttagtgtccactccaagttgttgagtcgcgaccgtccattggccacctgctgacaagctagcggagcggcggcatcccgcgatcaagacgatatctgacgaacacatgaatcgaggttcgcctgatcgatcgctggcctggcgcgacgacaacgactacgactacgccgtgaacctaaaagatctcgacaccgccggtggagcttcacacaggtaggaaaaaggggcatggaactgcgtgaaagtgagcctcacgcacccaacctccacgggaacatgatcgacggctcgacgccgccgccggctagagtctagagcagccggcgcccgacgcccgaggcgagcggcatggctgggcgagcgaaaaggcaggtaggggtaggcgtagcggactatgggagggacaggggagcgcggtggtggggcgcatgtggctttcacctcctggccacacacacagacggcgacaccgagctgagcagagctgatgccgtggcccggggaccatcgctgcggtgctgcctgccgcgcccgcgctcccgcgcgctacccgtcctcctgtgccgtagcccctgcctgttcagactgcgcgagccgggatgcgcgcacgtcaccggtttgcggccggcacagacgcaaaccacccgattggttcgctgcacgcgagaggccaaagctttgcacgagcctggtcggggatac

SEQ ID NO: 7 - DNA, 993 bp, Zea mays
gtaagcctacattcagatctaaaaaccttcactctaggagttggatgtggataaagctacgaaccatacttgtaaccatctcactaaccaacccaaaacttctgtacctgcatcgtatatgtgtttatcgtgaatgtatctacttgtatttatatatcaataaaactgaatcaaaatcagcagcacgtcgttgaacaaacaaggacaaagacacactaaactcatccatcaattcttccactcatccatcttcttggaacacaatcaaggtagctaacaagctccggagctccaatccgtgccttgcctgaacggagctgaaccgacctcatcctagtcggtcatgtatcgcaagttgcaaggcaaggtcataaacctagcaaaaataaacagaacaggcccgggctggaagcagccgagctggacgcccggacccctcgatggaaaaagaagagaaggggaaaaaggggtaagaagctgggcctcgattacccaccccgaagcgcttcggatcggtcggcggtctcttccccatcgatcacgcgcgcggccgcggcctgcccgaaccgacgccgagcgacacgccgggcgatgcggtcagcaacccggccctacactgcaaaggaacgcgagcgtcagaacgtcctgaggcagcggcggggtcgggggcggaggtggaggaaggggatccggcgaaccctcgaggtcgcggcggtggacggcgacgcacgccaagcgggccgttactgggttggcgacggagaggggaacggacagaaaagggtgttggccggacaccctttttatccagcgtggcatgggcgcttttccacgtggacgtacgacgctgcgatagacacctggcagataatatctgacgggcggatggacaggacgggcgggccgagcgctggcgccagcaggccgcagtacctggaggctggggccccctccgtggtcaggcacgccgtcgcctgtcggagtcggactatc

SEQ ID NO: 8 - DNA, 484 bp, Zea mays
gaaatggctgttaacaggcacgaaatgacccgaatcgaagcctacagtcaccactcgactgccaatcacacgaccaccaatcccttcccttgtccacggcctgcacgaaaagttgtattcattgtccacggcccgtaggcgaaggcgaggcccatttagctttatgtcttgcctagccagaaaccctaaaacgccaccaatcccttcccttatccgctgtccgcctgtccggttctccctgaccctgagccgctcccgtagtcccgtcccaagtggccaggtccagctccaggagctccaggcctccagcgcgcgtcgccgccactttacgcccgtgcccagcgccccaggtagcttacgcccgtgcctagcgccgccaccttacgcccgtgcccagcgctctaggcccctaggtagcttacgctccaggcctccaggctccagacctccagtgcccaggatacatatttgattatatgtttcc

SEQ ID NO: 9 - DNA, 2273 bp, Zea mays
cgaaaatagagggtaagttaaaatctgtcatagaatagacaatctccggcagttctgagaatcaagacgttggctttatggaagccctgcgtggaagtgggattagacaatctccggcagttctgctattccatttcttatcccatcttatttgaagctctagtatgcaagtggcaaaacattgttttgtatagacatatacacaatcgctggacacagcccagcctacgacggggtcgtactctactcacactgtaaacgaaacaacgcaggtctttgctggtgccgatgattccggtcaagagtcatcaaactccctgtctgtaatcgttgcaattgctaacgtgtgcatcgtcattcgacaaaaccgcaacaccgtaccccaggcagcattcttcttcgcgcaagagcagagcttattttaggaagcccgcaaacctcacctctgcaacagccagcaggcggaccaaacaacagcctgcactgcagaagctgcaatcggcaccatacacgtaccaattcaaattcggctgcaatggcggggaatttaatacgaatgcatacacagcgcccatagcgtacgacagcagtagatccaatcagcaatcaccactactcatgccaaattgtaaagtttcaggcgctatccgatcaaggacagtgctaaaattaccgtcggtgctagctgctgccctgcccaatctgttaacgttccattttattcacccgtttgactcgacgaaggtaggcactatggtcccacatgtcatccatgtgcatgcatgccaccacatccgttattgtccattgcaaaatcgtgtagagagaagcgggccacgacagtctatctagtctttgattgcttcttttctgaatgaataccacggtacggtatcccccgtagcatgagcattaaaactttttaaaaaagagagacagagagaagaggttcaataatcaatactacagaagataacgtaacatgaccccttgtagcctattttcccccagattagccggtgcccgcggtcaccccttcaaacttgcttcgccaatcgccatggggggtgcggcgtgccttgtggaggaccatttgcattccaatcgcgctagatgttcgcgccatgcgctggcactggcagtggcaagcaaaccgcgctccttcaaaaaataaaatgccgcatttcgctgctacgaacaacggacatggctggcccagcgggccttgtgcctccttcctccggtaaccacccaaccaacctcaataaaacagcacccccgatctccatccgtgcgcaggtgtcttgaaaagaaagaagcttctccgttcgcacgcagacgcagcgactcgcaggcagatagatgcggccaggccaggcgcccggcacagctggagcgctttgctgcgcctgcacggctcggctcggctggccctggcctgcctctgctctgcctgcctggcacaccactgccgctcgcctgctccagcgcagcggcagggcacgtgcgcgcgccagccagcccggcgcgatccgtctaatcgccgcgattgggagggacctgctctggctgtggctggaatccgagccggtgggcgatggcgcacaacttgcactgcagtactagtacgagccacgtgggtgccgtcccgcccggccccattagctgtgcccagcctggacggagtacggacggacaccaggagtccaggacatggacactcgcactccgctccgtcctctctcgcgtaacagcgcacgcctctcatccttcgctgcctcgtgggggccacaggcgggtgctctggtctctgtgggcgacgccgagctggctgaccggaccggattagttaacggcagccggggaatggaattaacgcggcgtgtttttataattgaggtggcggataccatacgtgcaggcccgccgtggacggagctgcggctgctggacagcacagcgtcggcctttttctttttctctcggtactagtacaaagtacaaagataaaaatacatttgttttcgagatcagcgatgagtcatcgccatgcagtgcgcgctgttcactgcacgtccactccagcagtgggcagtggcccgctggtggcgttctcccgctgagtcagccgagtccgcgcccgtgcgtctctgtctcgtgacgaatccggccctctgtggctgtcacatgacggatccggccctcggacaccctggattagctccagtatttaaagaaaggttggtttagtggatgagggcaccagctgagct

SEQ ID NO: 10 - DNA, 1213 bp, Zea mays
tgattcactgatatccaacatgttatcagcacgatcgcaagattacagaagaggaatctgaaactcgctagatggatcgcaggggccatgctctcacttctgacttgaggacagtaaccagtgcggttggccattgttgatgcgacacgaggtatatcagtcatccatcatcatgattaatgattaaggctagaaaaattgcctctgcatcatcacgtcgccgagaactgggaaagtccttgtgcgcgctacaggtcgacgtggccagtggggccggcaccgccaaggccgtcgcggctatgcccatgccgacggatgccggaggcagtcacgccgtgggaatccacaccgtgcgcgccacgaagggcctgcgcacccgtgtcacgccgccggaccgcggtttcgcgccatggccgtatctgcgcaagggtacggcacccgcgtgccgggggctgtaaccgtacgcgcctgggaccgcgcgcccgtgccgccacacctggccgttacagcacaccagggctccgtcgtcgcgtgcggaggatgcggtcgtgcacgcagatcggggcctaggccggtgcccgcgcgcctgcgctgccagggggctgtgccccgtgcagccgagcccggaccgccgtcggggctgcctcaccgcgcccgccgccgaggctgggggccgcgtcgcctccggtgccgaggctgggtgccacgcacccgcctggctgcctgcaccgccggagccgccaggggcgagaccgtgaccgctcctggtcgggccgaccaccggcgagtcggggctagggcccgcgcgcgcctggccgaggccggggaaggaggccgcgcccgccgctcctggccgggccacccgctggcgagccggggctgggggctgcgccagccgcaatgagaccgcgccatggccgggtctggcccgtcgccggggccgaagcgctcggctgctcaggctcgagcaccgcgcgaggccgcgctgtggcctgcctgtgcgcccgcgtggcagcggcttgcgcacccgcgaggcggcggcggtggcggcgccctgctgctggtggaagagagagagggccgcgacaagacgagttaggaaaccctaactgcttgcccacctcctataggccgcaaatgggctggctgttttggcccatttcgacaaggattattttttttggctgaatatatttacaattattatcatcac

SEQ ID NO: 11 - DNA, 1393 bp, Zea mays
gcctgggccgccactgctcattttcggcccacttcattaacccgccgcgtggcccgattacccccacgcccgcgcgcgcagtcaccctaccgggtccttcgctgaccagcggggcccaatcgtcatcttcttcccccacttactcgacggctcgacgcagacccaatctcaacagaaatcgccgcatgactcggatcccgctactgcttccctggccgtgcggctgtagcgctactgacgggcaggcccgctgaccaaccttcccatcgcgcacggacttctttcgcacgcctagcgagtggagcgcggattggtcatcgggtgagatccgctgcaagctcgtcttcatcctctcgtgattaccgtggactccgcgcaacagaaatccaagcatcgcggcctagcaaatcccatcagagttcgattccaacctgcatataattgtgccctcatactaccgcacccagatgcataggacgttcgggtcttcgcgtgcgccagggagacatctaccgccatgaaacagagagaccgaaggaggaagaattgttgttcctgtttcttgggattcttctactattcaggcgttcatccctctttctctcgtggcgacgaagacgaccgacgccgacgccctgcgcctggtgccaggttccgtcgggtcttcgccggcgatggacatccaccatggccgactggcgagagagagaaaggcaacaagagcgagagaacgggtgtgttgccatcgggagggagttcaacggagccgtgcactggacgtccaagacttccctgtgcgtgcggtgtagagcagacctggcctcctcgccgggcgtgaagactgcgcgaacgccgtcgagtcgtggacctgggcgtgcttcaaagagcgccgcggtttggccttgccatcttgatcgaccggtaaaattctttccaccctgttcgcattaacaccccctccgcgtagcactcttcagatccgggttttgggaaccagagagggaaactatgcatctccggcgaacttcattgtcgtgagcttggcagcgccgccgcgcgtgggctgcttgatggggttgtagctggagaaaggaacagtggctgtcggataggcgatggatggacgagattatggcatcgtataccccttcgcgcgatcggatttgggccgtagatgcggaatcgggccgttctgatttggttagggcgtggcaagtcatcgaccgttgatcccatatccaatggacctagttgcatacagattcgttatatttgagatctaaccctagcccttgatttttaatccaacggctcagaactacagataccctttcggcctggaagacttgcataagagccactctgcttttctagaataaacccgcagtccacg

SEQ ID NO: 12 - DNA, 1201 bp, Zea mays
ggcccaactctgccattggtgcctctcacctgggagcctggcatgtcggcccagcttgtcagggtcgtcttcaagctggaggctcacccgagatctgctcggtgattttcactacttcgtccacctttcccctcggccatgtgcgctctctgttgcccggatggctgaccgcgagatgggggcgctagacattctttcccctctcaatctcgcacatggagtaaacatcggggttgttttgttgtggtaacagcccaaaaatcccgcctatcaagcacccttaggctcggactgtcctaccccggtgataaatacctgggtacggcaccccccttggcgaactcctgtcgagccccgccttagagcacgtcgttgcggcgagtagctgggaaggagatggagggccaccatgcagggtgggtgaagatctggttcaccggcattgtgccctcggcggttggcagggggtcttcctcgtgtctcatgggggacgcccatgacctcatcaggtgaacttgaaaccggtggtgcatggaattgctcaacggaatgtatggccaccaccatgtcgcaggtcctcgaggccatgatcctctgatgctccagtccttaggtaaggtccccagacatcttcgctattttcgtagcaccgtttagaatcaatcggatcgggggtttgatcctgggtgcacggattcggctttctctggcgggccctccaccgtgggcagggcgcctccaccgtcggtaaagttcgtgtgggagaagacgcgtgggggccatcagatcgaaaatggatggacacgataagatctcgcgtatcgttcggacatgggggaatggtgaccgtccgatcgtcaggaacaatgtagatttgactaggtatgcgctctgaactccgcgtccttccacctttcgatcttgatcggtggggcgtgggtaattaaggcttctataacatcctgaccgtcgatcttcgaatcaacaacctcaagcgtgtaccgcttgggttaaagccagatctaatccacaccattgggatcggatctgacaattgggagcacgttctggttcgtctggggtccgatctaatcctgacggtgggcttgtgatccaacagtcctagtcgaagcgtacctcttcggccatgtaaatttgcaaaagaatccttaaatgttttaagaatcaacccgcgctccac

SEQ ID NO: 13 - DNA, 1195 bp, Zea mays
gacatgccaggcggaccaggcaccggcccagcctggctggccggcccaggaggcccaacaagggtccaaaacgcctattatgaggggcggctagggtttcaaaccctaaccgcccacagctcctgcagtagccgccaccagggagccgccgccccgtccgaggccggactgtgccctggctggggccggtagccgcgaccaggcaaggggcgaccgcgccctcgccgggatccaggcgctcgccatgcgccgtggtccgcgaccgccttggagagccgccacggcctccgtcgggccgcccgcagtctgctccatccggcgccaggagcagcagcgcctgggcgtcctcgtcatgcgtgccggtgtcagccaaccccgcaccaggaggcggccacgccgaactccgcgagaggccgaccgcgcccgacgttgccggatccgcaccggcggtcggtgtggcagggacgtgccgcttgcgcagagatgcagagcacccgttcaggcgtccacccgttgacggccgagcgtccgtgcccacgctggcaactggccgcgtccgagccgggggacggccgcagcgtgcccgcgccggggcgctcggcggccggggcgctcggcggccggggcttgcccgcactggggctcgaccgcgcgtaaccgaggctgtcccgcgccgtggtcggttcttgccggactgcgcttggtcgcgagcggccaagggctcgcaacggccgtgccgtgtccattggactggccggggtcgtgcctcacccgcgccgaagtgcacctgggcacatgcggccaagggctcgcagcggccggtgccatgtccggtgggctggccgcatggcttcgtgtggggagcctgcatggccctccctgtgcatgagccgcatggccctgtgtaggcaggcagacgcaccgcgcatggccgaggcgcgctgctctgcgcatggccttgttgtgcatggcccgcaccgtcggtgcgctggggacagggcttgtccgcgccgagccttgccgtcgcggcgccgtccggtgtggtatagttgtggccaccgacgacataaaaatttcacgcatacagtgaaagcggagaggaagagtaggttccataccattatccttctccaactaggagcgatctgttttcttcctaccagcaatttctctccgttttctcgcgagtcgtgct

SEQ ID NO: 14 - DNA, 1176 bp, Zea mays
gaacctaaggttggaccagcacgcaacgctatctgcaggatcaaagatcgacaggctagattttaaacaccaagatccactccaatccgtacgatctgagatctatatttgatcccttcgaagcggtcctaattatctaatcaagaccgatcagatcgagatcgacgactcctactctctctcccttattcccctcaatgccttcggaccgaggccaccatggaggcgccatggccgagaccggactatgagctccctcaatgaatccccttcaaattgaaacttagctacacattgctgagactaaggcgagtagttctagggctctcttacctggagttgggacgacgaggcaccccacccacgtgttctgcggttcggcactggaaggcatgctccggcgacgaattcctctatgcccgaccaccattcgccttaacgcacgctgctccccacttcggtgccctccgtggaacgattctgcgcgcacaccgagggcctgacgctgccatacgtgaatctatcatagcgactgcttctctatgctctttctctgtttctactatggcgccgggtgggtttgctctcggctctaggggatgatggggggttgctcggcgtctggttttatagaccctagcgtgggcaagaccgaaagactcggggtcggggtcggggttacccattttctggtgaagtttgttgtggcatctgtgcaatcttgtctaggaagaagactctggacagctgggcccgtttatcagggttaagggaacgtggcgcggagcggctgattaggctgacgacccggccccagtcgtcagcggcaaaagacgttcgggctgaggaaagccagagctgacgaggcggggcccgcgcgccatacacactggaaaaagaaaaaggagggaaacgtaatgggccagcgaggcatatttggcccatcaggacagggtggacgccgagagggagttgggtcgaggagaggtttcaagcccaaaacgaggtatgtcctctctttccaatttgtttagatttcttttagtgttctaaattctaacttcaagttttgatttaatctgccgttaaaattttgaactcaatttaaatgcgcaaccaaaaatacccagcatgatgcagcaatattcatatattttgcattattttaataattta

SEQ ID NO: 15 - DNA, 913 bp, Zea mays
ccctttgtcgcgtgcggcccattcctcactcgccctgcctcaccgacgtgtgggtcctagcgctcctcctctcttggtgtctatggcatgtgggccgtctggccagaccgtttcaacacgcaagggcgcaagcctgtcgcgagtcttgtacacgctgacatgtggcacccggtcgtcatcactaacccctcaatcgaagacaccacaacagaatagcggcgccaggtttctcggtcgggccgctccaaccgccgttgcccgggtgtgcgggtcagactccatgctataaagagctgagccccgtacccctggaacccttggaccatctctcgccacccagaacaaggtaacctcgtcgccatcaccatagttgtggagcaccgccgcagttggcgaggtgagatcccgcggggcctcttcaatccgagtctgggggtggcaggctccctagcgcaaccaagtgggttcggttgtggggaccggtggcacgcggagtacctcagagtggcggcaattgctcgccgatgcatgccctctggtgcggactacgcctcttcgtgggcagcgaactccacatctctgacttaggtgagacccctctccgcatgttcgccatcttcccctctaggtttagcaccggacaattttaggtttggggtgtttgggcatcggattgcgtgtccgccggcgaagctccatcgcggcctcgcgggtatgccatgcggcgctgtcgtgttcttctgggatgggagatatggtcacgtcagtcgattgctcgcttaacggctgcaattagatcaaatgtaccccttcgcgcataacgatccgggttgccgattcttgatcaggcggtcaaaatcaaatccggatatcggtttgggtaggtgtaacataagtcattcgattcagatca

SEQ ID NO: 16 - DNA, 1149 bp, Zea mays
aattttgaagaaaaacaataaaaaaggggataaagaaaaagcttaccttcggttgggccggcttcctcatcccagggcccaacttggcacaccagcgcggcccactcagtcctccccaccttttgctacgcgacacacgctgacaacagggccccacgcgccagtccctcacccccacggccgcagcgcgccttgtctcgttgacgcatgggtccgagccgtcagcttccttttctccagcgttgtagatcccgcggcgcggccgccgtacctcggaccgggttttcgcgtgcgtgaatctgtctccgtgtgtttctgccaccgtgtgggaccatccctggccatgctgtgccccatccgtgcgtagaaatcggatcgaaacagactcttccccgctcaaatcggggatcgcaacagcattgggcggggctctcggaggctataaaaaaaccgtgtccttgggctctcttcctccgtctcatcctccgtgattgccatccatcgccggcgtgtggctctagacatcgcaatttctcatcggtctgatgcgggcaccaccgtgtcgccctacccgtggccagcgctcgtttcgtcgcggttcgccggtaagccaccccatacagttccctggcccctgggcatcgtgtcggtcacttcggagtagaggatagggctagatcgtctccggcgatgtccgccaccgtctacacccacggcgccaccgttcgtcgcccgcttggggggggctgaagcggtgtgtgggattgtccgtcgtcggatgctcgttcgacgatcgggatcgggtgctggacctgagaggatatatgaatctggggtcgtccgatcgttgttgagcggatgagattagatcgcacaaacaatgcttcaggtatgtagatctggagcattggtctgcgggctgccagctaggattaggggtcgggtaacgattcggtatggggccatctcgaccgttgatcggctgacgttcgacccagaacggaacatgcaatgccgctatgttgtatttgctaaagagacccccgtagatagttaaatcaacccgcggtccactgctgtgtaaaagcaattacacttgggtcttgaaattcacagatcagaccctggggtttataagaatcgtgtccg

SEQ ID NO: 17 - DNA, 2363 bp, Zea mays
tttgtatggaattgttgatcccacgctctttagttgcaccggagcacatatatatttgtatggcctttttctccgtttgcctctccgccatcgagcctatatgcttaacaagttgatacaactgttgcttcgtaataatagagattagacagggttgttgtatatgcacgtccatttctctattcgattttggaaatatgtggcgtcggcgaattaaccatggaatacaaaggctatataattaattaacctgtatatatgacacgttaggtaataatataatgcatcgaatgaccaatatatggtacagtgaaatgtaattatggagggagggagggaggtttttgttttacatgtcatcacagattcgaaaactgtcacgcatcaaactttttctctgcatatatatgctacaaagtagctaggaattagacaagcaggttgtcgcacatgcacctagctgagacgttgtcagatgattatatattatgcaattatgcacgtacgtccgtaaaactttttttgttctctctcagcatgcatggtagctagagatgactagctgcagagtagcaggaacgttgcactagctagggtacgtacgatatgaaatgggctttcggccggggtacacgtgatataggccggccccacggccactgcaaggccatgcatgtttgtcgacttgacattatattatatatattaccctaggcaggctaattattccaacaccgattggttagcaatacaaaaaaaaaaaatcaatgcccagacatgcacgaacacgacgatatctacatgcactccgacatgtacgcatgcatggtccaccgtccacactctcgtgaatatcgagacagcatgcgcgcgtggtaggtaacgattgggaaaaaaaataatgcgtgaacgaacggtgcgtggatatgacacaccaccaagacgtactgagacatactatttgtggctactaaacaatgtactcaccccatcctaaataagatcccaaccgtacgttgctctctgttggagcctagtgtgttggttggtggattcatccaatagggcgcctaaacaaataaataatactggcaggttcgtgcacgttagatctgtgtggtccaccaaacaataatatatatgcaacaaataaataatattattgtgtggtccaagcaataatatgatgtgacccggccgggccatgtcgtcgtcgtcgatcggtccctccatcacatgcaatgacgtgttaagttgtcggaatatgctgcgtttggtcccgtaaccatctatcgtattcctcagctagctctcacagactatatgtacaaaacacaacaagaagcaaggaaccaacagtgagattcgaggtgtgtggtggtgacagcggcagcttggatttactctgtgtccgcgctcatattatgataattaattaattaattaattattgcatcgatcgaccgtgcatgtacgcacacgtacagtacagaggacataaagaaagaaagaaagctactccacgagcgcacatgcatgatgttcgttgcccatcgatgtcaagctaggcagagtctctgtctcgtgtgattattgatcagatacgacacgtttgtgtgcatgaaaaaaaagctgtctcggtatggtgctgggccaggagagacagcagtggctacgtatacgtattatagcatagcatagcagctatattatattgttggagagctcaccactccctcaattctctcgatagtgattttctatgacactatgataaataaatcttaaaccagaacattattttaaatcaaagtggctatataataatactctacatatatggctagctagctaggcaccgattctcttcaacattatatatatgtggctactcataaaagcgggttgttacgagcatatatggcgattaaattaacagtatactctgaagctggcgattcaattcactgttcatccgctatatcccctcctcgtcgcgacagaagctgcgatggccaccaggggtgacgtggaggccctcagctgcggcggcgcggaggagctcgaccggtcaggagaaccaccagggcagaggagctcggccggcgccatggaggaaatctgcggcggcggcggcacggagaagcttgaatatgcgggcgcggaggagcttgaatatgcgaagaagctcttcggcagcatggaagcgtcatgttgttagccctgctattagggtcaaacccacccaaaccctacaaatgagttattttcttcaaccaaacccaccatataataattaaactaaataacccacctaaatccacccatagctactgggttaaacccattacctaaacacatgg

SEQ ID NO: 18 - DNA, 1449 bp, Zea mays
cgctagatgggcatgttgatccttgccaaaacgctcacacagtaggtgacaaaacacataccatgacatgtacactaatttttgtttaacagattgcaaccaacaggcagctgcacctaaaaaactcattgtagcaactttgatccacaccctatgactcattccatacatggcaaaataatcttcataatgtttctgccaaagttttgggttctcgccatcgaaactaggaaagttaaggagtggtaattttcccaactccaactctaaccttgcccttggtggcctaggatcatgatactcatgtggatccagatgagaaagtggagggtgaaacatgaaatcgggcgtaccattgatcgggagatggttttgggcaaggagatgcccaaaaccatgcccccagcgatttttgtcgaagcaattcccatctgggctgtcggtgttgtacgcggcagcaggcgagcgctccgccaccgactggtacgggccgagaatacccgcggcggtatcgagctgtggcggcggcgcttgggcagcccacttggacacctcgtcacgcagaagatgcacggaggtgtggatctcgtcgaccgaagcctccacgtcgggccgccacgactcgaaggccgcgtaagccgacacgagggcaccgaggcgcattacgaaagcggcctcgacttcatcgaggcgctcgatcgctgcggtgacctgggcgtcgagttccacgtggatcgatgtcagactctcgatggcgatgttggcttccagatgttgcggtgacgcggtgttccacgcgacggttgtctcgagcgtgcaccaacgcgtctcctgagcaaagaggtggtcacagatgtcgcgaagaagcttggattgttcctcgatcgccatcttcaggttgggctccatggatccggattccttcgcgcgacgcgtgtagtaggtgtgtgctagcgcttagatcagtgtctccgatatcaatttgtgagcgtccgctccaagcaacataatctaggaatgacgagagtaggagaagggagattctagaggaagaaggagatctgatagggaggggaagtagttgttcgatatatttctctgcctaaaatcgtcacagtgcctgttcacagtatttaagtatcaactattacaaggctcgacggcccattatgccacccagtcctctcctaggaacttggcattgggcttcctgatgcggcggcccaactccagcccagccacgtccgacgacgacgcctgcttcttgacgcacgtcacctgggcgatgctgagagtgctggttacctgtaatcggatacaacgtaatcaatcggatacatgacatatatttttcttagtgctcctccggctccgtgatcggatcacgctgctgcatcttataagctgaaagaaactgggaaggtgatgagttggaccttgtgctgcccg

SEQ ID NO: 19 - DNA, 1105 bp, Zea mays
tgaccagcattgatacacctgatatgtcaatcgcctttaacgaaacaagaataagaagcctctcgggggtagcaataagaaactccggctcacaagttttcagtccagagatctagtgttcaatggatgcgccaggatgcaagctcactgaatgaatcccaagaggcttcaggggtttgcatatcgatcgcacttgcgcggccttatcctgtgacgagacgaggtacagcaggaaggggcaagacacgaccatcccatgcctctctttcctagcgacgatgtcgcaggcagtggagacgagccacgcggtctgcgtcaccgtggcgcacgcgccgctagcgtcgagcacgtcggacggtgcctgggtggagcaacacctccagagctccatgccccggtcaccggcgccatggatgctgccgccgcctttctcgggcgccaccgcatcccggatagcgttgaggcagaccaccaggaacttggacagccggtcgaagtccccgtggccctcatccacgcccgtcgcccttcgccatctcggctgtgctggctaggtcgcttcggcgagatagccgccaaccgtcggggagtggaggggtgggaggtcgacaggagcacgagcagttgggcggttcttggcggaatgggagcgcacatcacgaagagtgcctaaagaagatgcacagttgcttagggtggccggtgggcccggcggcgctagggcaggcacaacgctcgcccatgcgggagcgaggcacgatggccagacgcgttctcgcgagcgcactgggtgggtcgcggttgctgggccagggagggggcgctggcctggcggcctgggcgtgggcgcctgtgcgctgggccacgcggggtgggccggccacgcacgctgtaggagcgagccagggacgcggggcagctgggccgttggcgcgcgcagcggcccgggagcgcgagcaggatgcggggggaaggcgaaggcgcgtgggccggggtggccgagcggcccaggaaggggggggaacgggccacgattggggaaaatgaaggggaaagaattctgggctttttcttttatgttcctcttcttttctttgtccatttc

SEQ ID NO: 20 - DNA, 280 bp, Maize streak virus
ccgacgaccgaggcttgagctggctcaaggcagaaacgggagctcccaaactctatcctaccggtgcgccttcgaaatccgccgtctgccgtcttatatagctgatttatttgggccggactgggccggcccagcaggaaaagaaggcgcgcactaatattaccgcgccttctttttctgcgagggcccgaagggaccgagcgtctttgatttaaagtttggttctgctttgtttgattttaccaaagctgccgcaactaaagacgccggtccaggcgac

SEQ ID NO: 21 - DNA, 1102 bp, Zea mays
catgtgtgttgtgcgagaccagccacgccgatacagttactgtgctcacgataaaattgaaaaggacaaccccattgttccagtcatttagtcatggaagaaatgatcctttccaacccaatctcctagccttttttcttcgaaagaaaggaagatctgcccgtttttatctattcccgcccgcaacaagatcgatgattacccaccagtaaaattccgagaagcctacacctaccacaaacttcctctttttttttttggttgttgttgtttgagaaaagaagaagtccataaagcaagattcccgtaggtgggcactgggcatgcgatgcaaggacaggtccatcagtccatccgtcagtcagggaatagcagctgcggtgcaggagcacgcacacccagctccctccctcccttgttcccaatcatttgctcgctctacgtgctcctgctcttcaccgtcggagggaacgggtcttgtctgacggtacagtcctcagcaaggattaccggtaaacacggcatcgtttatctgtaaacttaaactagcactggatttaacaagcgcacaagctagtggacactgaacaggccccccggccgtgccatacaatcaggtggtaataatgcagcaattatgcggctgcttacccctggggctggggggtgggaagccaataatgttacctcgctgacagaaaaacgcatagggaggaattcctttccggattcccttgggtccacaatcaatccccttgcttttccctcgccggtgcctgagtgactctgatgagctctttggcaaatggatgacctgtgtggaactctgatgagctctttggcaaatggatgacctgtgtggcttggccaaatggtgagtgagtggagcggagcagctgccgctcactcagctcctcctcgactcgccccgggcatcattcacgaatctgcttttcccccagccccggaaatacgtttaaaggaggccgttttcagggacgagatgaggttgattaaagcaaagcagagcagagcagagctaagctttcttgattccttcccagaatgtcgctcgagctttaggcaggcaggtgactgaaaa

SEQ ID NO: 22 - DNA, 549 bp, Zea mays
cggcgcttgggccaaatctttcccccctcgacctaggtagaatcacctgcaccggcccatctccgtggtctccacggctcaaactcccgctgtgctggtgcctttggtgactgccagggcagacccactattcagttccacctctgcgccaatgactgatgggtctgcctcatcatccgcggttccccatccacgtgctcctgctatcaccgtcgtgtgggccatgtttgtcagtattctcctcaagctcaggatcttaaccaatcccgcgctgctcactgatatggttgggaggcaggtgctgggatccctgacctcggtgcattgggcgctcaccttgtccctataaattgtcgtgaccgtccctcgcattgaaaacccaagccgaagtaccgacaccgtgattacagagattgcacaaggaagagcagggtgaagtccgccattggaatctgccgtcgtcaactatgcatcatgggcatcaccgtcgaaccatcgggcaccagccggggagcttcgccatggcgcgaggaacgttttcgtggcccc

SEQ ID NO: 23 - DNA, 92 bp, Agrobacterium tumefaciens
tcaagagcggagaattaagggagtcacgttttgacccccgccgatgacgcgggacaagccgttttacgtttggaactgacagaaccgcaacg

SEQ ID NO: 24 - DNA, 331 bp, Figwort Mosaic Virus
atcaatgaagaatcttcaatcaaagtaaactactgttccagcacatgcatcttggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactattgcgtttgacgaacgcagtgacgaccacaaaa

SEQ ID NO: 25 - DNA, 293 bp, Cauliflower mosaic caulimovirus
acttttcaacaaagggtattatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggttgacgaacaatcccactatccttc

SEQ ID NO: 26 - DNA, 416 bp, Cassava vein mosaic virus
aaaattatccattccagaaggtaattatccaagatgtagcatcaagaatccaatgtttacgggaaaaactatggaagtattatgttagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgag

SEQ ID NO: 27 - DNA, 401 bp, Chlorella virus
caacggagtggtcgtacatactacaaatggattgccaatagtattcgcaatggatacacaccgaggttgttgcgagaaattttgtatcacggtacaattaccagggggccttacgcgatatgattttattggcgccacgattacgaaggtaagatttggtaaagaaaaacgcaaatgcgatattaatttttcggaattaattatagaaacttcggtaggaaatatcgttttactggcagaaaacattcataatggatattactctcatgatgtattcgcttgttttgaaggtaaagttgaaacttttcgtttgtaaatacaaaaaatgtatatgagtatttgttgtcggaatgtcatatcaacaatgttgtgtatatatgtgtaaactaaaatacactata

SEQ ID NO: 28 - DNA, 194 bp, Figwort mosaic virus
agctgcttgtggggaccagacaaaaaaggaatggtgcagaattgttaggcgcacctaccaaaagcaactttgcctttattgcaaagataaagcagattcctctagtacaagtggggaacaaaataacgtggaaaagagctgtcctgacagcccactcactattgcgtttgacgaacgcagtgacgaccacaaaa

SEQ ID NO: 29 - DNA, 351 bp, Figwort mosaic virus
gagcgcttgacggtgttgaattaatttgcaggtatagttcaggaagcttcaaacaagcagaaaagaattatcacagcaatgataaagaattactagcagtcaagcaagtaatcaccaagttttcagcttacctaacaccagtcaggtttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgta

SEQ ID NO: 30 - DNA, 381 bp, Mirabilis mosaic virus
ggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccaca

SEQ ID NO: 31 - DNA, 499 bp, Cestrum yellow leaf curling virus
aagccatcgaaaccggtccactggtaaaccttcaagatgaagaaagaaagattctttgccaaaaaatgcaaaagtacaaagggcacagctgtcagttgtgcaaatccgagtcatctggaccacaaacatcagaagagggtctacaagagtcagaagacgaagacttttcggtgctagtttaattactggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaat

SEQ ID NO: 32 - DNA, 371 bp, Peanut chlorotic streak virus
atcatctggagccaacaagctaggaagccgtctgagcaagatattcagatccttacagagtttttacttcggacagtcaaaaatgagtttaacttctcagccgaggtaaaacaagaaatatgcttacgtctacagagggatttctctgaagatcatgtttgccagctatgcgaacaatcatcgggagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcat

SEQ ID NO: 33 - DNA, 361 bp, Rice Tungro bacilliform virus
aagaatttgggtttgattactacagttgggaaaatatggttgatgaagacgaaggagaagttgtatacatctccaagtatactaagattatcaaagtcactaaagagcatgcatgggcttggccagaacatgatggagacacaatgtcctgcaccacatcaatagaagatgaattgatccatcgtatggacaatgcttaaagaagctttatcaaaagcaactttaagtacgaatcaataaagaaggaccagaagatataaagcgggaacatcttcacatgctaccacatggctagcatctttactttagcatctctattattgtaagagtgtgtaatgaccagtgtgcccctggactccag

SEQ ID NO: 34 - DNA, 291 bp, Sugar cane bacilliform virus
accaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccg

SEQ ID NO: 35 - DNA, 281 bp, Sugar cane bacilliform virus
gaacaccgttcgagtgtcatcgacaggccaaggccaacagatgatcatttcagaccttggggggatgttacatactggctgaataaagaagcagaagagtgccacacaaggggcgacaacgtcgaaggcgcagaagacgcagtcgatctcactgacgtaagcaatgacgaccagtggaggagatcgtaagcaatgacgtatggagcgtggaggacccatgaaagcactgagaaggcatctcaactttcggtgtgtgagtgcgcatcctatgcgatgctttg

SEQ ID NO: 36 - DNA, 500 bp, Blueberry Ringspot virus
ctacagaatttcattggagtaattaatcatttatctaatctgacagcaaacattaaaatgctgtataaatcagatacaatgattatctattccaaagcaacaaaggagattgagccagatcaggaagcagtatttattgaatttgaaaagaattttattgaaaataaaattcccaaaatgacgggggagatgaagaaagaattatgcaatcatatgaccaaagaagatcatccaggacactactgtccaatattgtccattcattcagcagggatgataagaagtcagtatcaagtgaagacgaagaaaagatcacccatggaagtggaagaataaaacgtcttcatccatcactatcaagacgtagacatcacaatgtaaagctacgggctaatattagatggcttaagaatctacgccaatgtaaagtagattccccttatcttttatttttaaagtttttaagtccggagttgagttccgtcctgtgggaattcctc

SEQ ID NO: 37 - DNA, 31 bp, Carnation etched ring virus
cgttgcaggacattcagaggcattaaattgc

SEQ ID NO: 38 - DNA, 129 bp, Carnation etched ring virus
ttggatggaccctaccacgattaaagaggagcgtctgtctaaagtaaagtagatgcgtctttaataattcatctactttagacgtcatgcatgacgtttaacatgcattgtatccagatcctccctggc

SEQ ID NO: 39 - DNA, 195 bp, Commelina yellow mottle virus
cgccgtcatcaatgacatcatcacagtactgaggagatgaatacttagccatgaagtagcgtgcgaatattacctatgcctttattcgcagcgttagtggcactgaaaggcataaagtttgttcgttcttatcaaaaacgaatcttatctttgtaacttggttacccggtatgccggttcccaagctttatttcc

SEQ ID NO: 40 - DNA, 159 bp, Dahlia common mosaic virus
agtaaacaaggaaggttagtccgatggcagatgtggctgtcccactacacattcaaagttgatcatcttaaaggtgaaaagaatgtgctggctgattatctcaccagagaatttcagcaaaaacgtcagcaatgacgtcaaatggccgattcaaacggc

SEQ ID NO: 41 - DNA, 275 bp, Maize streak virus
cgacgacggaggatgaggctaggggatggcagactgggagctcccaaactctatcctaccggtgcgccttcaaaatccgccgctccccctcttatagtggttgtgtttgggccggactgggccggcccgcgggaaaagaaggcgcgcactaatattaccgcgccttcttttcctgcgagggcccgcaagggcccgagcgatttgatttaaagttcagatctgctttgtttgatttatctaaagcagccaaatctaaagaaaccggtcccgggcac

SEQ ID NO: 42 - DNA, 278 bp, Maize streak virus
ccgacgacggaggatgatgctaggggatggcagattgggagctcccaaactctatcctaccggtgcgccttcgaaatccgccgctgccctctttatagtggttgtattctgggccggaccgggccggcccagcaggaaaagaaggcgcgcactaatattaccgcgccttcttttcctgcgagggcccgcaagggcccgagcgatttgatttaaagtttagatctgctttgtttgatttatctaaagcagcccaatctaaagaaaccggtcccggtcac

SEQ ID NO: 43 - DNA, 296 bp, Maize streak virus
atccgacgacggaggctgccgctagcgaaggcagggatgggagctctaacaaactctcctacagctcgccaagtttgttgccgccgcggagagagaaactccatcggcttatatagttgttctaatgggccggaccgggccggcccagcaggtaaagggggcgagcaataatattacctcgccccctttacctgcgagggcccggtagggaccgagcgtctttgatttaaagctcagatttgcttttgtcgtgaaatatcaaagctgccttgtttaaagaagccgtcccacgcgac

SEQ ID NO: 44 - DNA, 285 bp, Maize streak virus
atccgacgacggaggctcaagctaggagaaggcagggaagggagctcgcgaaactctatactgatacctcgcgccgatgaaatccgccgccgcccccttttatagttgttctattatgggccggaccgggccggcccagcaggtaaagaggcgcgcaataatattactgcgcctctttacctgcgagggcccggtagggaccgagcgtctttgatttaaagcttggatctgctttgtcgtattttttcaaagctgccatgtttaaagaatccgtccacatccgac

SEQ ID NO: 45 - DNA, 282 bp, Maize streak virus
atatcggacgactccgagcagcttgcgggatggcaggatgggagctccaaactctatatcaaccggtttgcgccttcgaaatccgccgctcccccttttatagtggttgtttttgggccggaccgggccggcccagcaggaaaagaaggcgcgcaataatattaccgcgccttctttttctgcgagggcccgtagggaccgagcgatttgatttaaagttcggttctgctttgtctgatttatctaaagcagcccaatctaaagaaaccggtcccctgcgac

SEQ ID NO: 46 - DNA, 289 bp, Maize streak virus
atccgacgacggaggctagtgctagctcaaggcagaaacgggagctcccaaactctatgtaaccggtgcgccttcgaaatccgccgcccctatttatagacgatttagctgggccggaacgggccaggccgggccggcccagcagggaaagaaggcgcgcactaatattaccgcgccttcttttcctgcgagggcccgcagggaccgagcgtctttgatttaaaggtttgatctgctttggtggttttttccaaagctgccgtgtttaaagaaaccggtcccgtctcac

SEQ ID NO: 47 - DNA, 277 bp, Maize streak virus
ggacgactctgagcagcttgcgggatggcaggatgggagctcccaaaactctatgtaaccggtgcgccttcgaaatccgccgctcccctctttatagtggttgtatttgggccggaccgggccggcccagcagaaaaagaaggcgcgcaataatattaccgcgccttcttttctgcgagggcccggtagggaccgagcgtttttgattttaagcgtggatctgctttgtttgattttgctaaagcagccatctttgagaaatccggtcccctgcgac

SEQ ID NO: 48 - DNA, 274 bp, Milk Vetch Dwarf Virus
ctggggcggggcttagtattacccccgccccaggatcagcggagtcatcacgtgacccgcacatgcctaatgaatacaatgtatataaccaagtggacatggtccccaccattatatttgaattaaatgcactgaatatatgccttgcttcgtctcgaagcaaagtaaggaataaatcggacccatatgagctgtatcccatgtgcatcgctttatttgtatggtggacattacatagcttttaagcggcgtacatgttacgcttattctttgt

SEQ ID NO: 49 - DNA, 493 bp, Artificial sequence
  Viral double enhancer
acttttcaacaaagggtattatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggttgacgaacaatcccactatccttcctcgagagctgcttgtggggaccagacaaaaaaggaatggtgcagaattgttaggcgcacctaccaaaagcaactttgcctttattgcaaagataaagcagattcctctagtacaagtggggaacaaaataacgtggaaaagagctgtcctgacagcccactcactattgcgtttgacgaacgcagtgacgaccacaaaa

SEQ ID NO: 50 - DNA, 1399 bp, Artificial sequence
  Plant-viral combination double enhancer
catgtgtgttgtgcgagaccagccacgccgatacagttactgtgctcacgataaaattgaaaaggacaaccccattgttccagtcatttagtcatggaagaaatgatcctttccaacccaatctcctagccttttttcttcgaaagaaaggaagatctgcccgtttttatctattcccgcccgcaacaagatcgatgattacccaccagtaaaattccgagaagcctacacctaccacaaacttcctctttttttttttggttgttgttgtttgagaaaagaagaagtccataaagcaagattcccgtaggtgggcactgggcatgcgatgcaaggacaggtccatcagtccatccgtcagtcagggaatagcagctgcggtgcaggagcacgcacacccagctccctccctcccttgttcccaatcatttgctcgctctacgtgctcctgctcttcaccgtcggagggaacgggtcttgtctgacggtacagtcctcagcaaggattaccggtaaacacggcatcgtttatctgtaaacttaaactagcactggatttaacaagcgcacaagctagtggacactgaacaggccccccggccgtgccatacaatcaggtggtaataatgcagcaattatgcggctgcttacccctggggctggggggtgggaagccaataatgttacctcgctgacagaaaaacgcatagggaggaattcctttccggattcccttgggtccacaatcaatccccttgcttttccctcgccggtgcctgagtgactctgatgagctctttggcaaatggatgacctgtgtggaactctgatgagctctttggcaaatggatgacctgtgtggcttggccaaatggtgagtgagtggagcggagcagctgccgctcactcagctcctcctcgactcgccccgggcatcattcacgaatctgcttttcccccagccccggaaatacgtttaaaggaggccgttttcagggacgagatgaggttgattaaagcaaagcagagcagagcagagctaagctttcttgattccttcccagaatgtcgctcgagctttaggcaggcaggtgactgaaaactcgagaccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccg

SEQ ID NO: 51 - DNA, 514 bp, Artificial sequence
  Viral double enhancer
aaaattatccattccagaaggtaattatccaagatgtagcatcaagaatccaatgtttacgggaaaaactatggaagtattatgttagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagctcgagtcaagagcggagaattaagggagtcacgttttgacccccgccgatgacgcgggacaagccgttttacgtttggaactgacagaaccgcaacg

SEQ ID NO: 52 - DNA, 713 bp, Artificial sequence
  Viral double enhancer
aaaattatccattccagaaggtaattatccaagatgtagcatcaagaatccaatgtttacgggaaaaactatggaagtattatgttagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagctcgagaccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccg

SEQ ID NO: 53 - DNA, 803 bp, Artificial sequence
  Viral double enhancer
aaaattatccattccagaaggtaattatccaagatgtagcatcaagaatccaatgtttacgggaaaaactatggaagtattatgttagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagctcgagggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccaca

SEQ ID NO: 54 - DNA, 499 bp, Artificial sequence
  Viral-bacterial combination double enhancer
caacggagtggtcgtacatactacaaatggattgccaatagtattcgcaatggatacacaccgaggttgttgcgagaaattttgtatcacggtacaattaccagggggccttacgcgatatgattttattggcgccacgattacgaaggtaagatttggtaaagaaaaacgcaaatgcgatattaatttttcggaattaattatagaaacttcggtaggaaatatcgttttactggcagaaaacattcataatggatattactctcatgatgtattcgcttgttttgaaggtaaagttgaaacttttcgtttgtaaatacaaaaaatgtatatgagtatttgttgtcggaatgtcatatcaacaatgttgtgtatatatgtgtaaactaaaatacactatactcgagtcaagagcggagaattaagggagtcacgttttgacccccgccgatgacgcgggacaagccgttttacgtttggaactgacagaaccgcaacg

SEQ ID NO: 55 - DNA, 823 bp, Artificial sequence
  Viral double enhancer
caacggagtggtcgtacatactacaaatggattgccaatagtattcgcaatggatacacaccgaggttgttgcgagaaattttgtatcacggtacaattaccagggggccttacgcgatatgattttattggcgccacgattacgaaggtaagatttggtaaagaaaaacgcaaatgcgatattaatttttcggaattaattatagaaacttcggtaggaaatatcgttttactggcagaaaacattcataatggatattactctcatgatgtattcgcttgttttgaaggtaaagttgaaacttttcgtttgtaaatacaaaaaatgtatatgagtatttgttgtcggaatgtcatatcaacaatgttgtgtatatatgtgtaaactaaaatacactatactcgagaaaattatccattccagaaggtaattatccaagatgtagcatcaagaatccaatgtttacgggaaaaactatggaagtattatgttagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgag

SEQ ID NO: 56 - DNA, 479 bp, Artificial sequence
  Viral bacterial combination double enhancer
ggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccacactcgagtcaagagcggagaattaagggagtcacgttttgacccccgccgatgacgcgggacaagccgttttacgtttggaactgacagaaccgcaacg

SEQ ID NO: 57 - DNA, 678 bp, Artificial sequence
  Viral double enhancer
ggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccacactcgagaccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccg

SEQ ID NO: 58 - DNA, 803 bp, Artificial sequence
  Viral double enhancer
ggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccacactcgagaaaattatccattccagaaggtaattatccaagatgtagcatcaagaatccaatgtttacgggaaaaactatggaagtattatgttagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgag

SEQ ID NO: 59 - DNA, 876 bp, Artificial sequence
  Viral double enhancer
aagccatcgaaaccggtccactggtaaaccttcaagatgaagaaagaaagattctttgccaaaaaatgcaaaagtacaaagggcacagctgtcagttgtgcaaatccgagtcatctggaccacaaacatcagaagagggtctacaagagtcagaagacgaagacttttcggtgctagtttaattactggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatctcgagatcatctggagccaacaagctaggaagccgtctgagcaagatattcagatccttacagagtttttacttcggacagtcaaaaatgagtttaacttctcagccgaggtaaaacaagaaatatgcttacgtctacagagggatttctctgaagatcatgtttgccagctatgcgaacaatcatcgggagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcat

SEQ ID NO: 60 - DNA, 796 bp, Artificial sequence
  Viral double enhancer
aagccatcgaaaccggtccactggtaaaccttcaagatgaagaaagaaagattctttgccaaaaaatgcaaaagtacaaagggcacagctgtcagttgtgcaaatccgagtcatctggaccacaaacatcagaagagggtctacaagagtcagaagacgaagacttttcggtgctagtttaattactggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatctcgagaccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccg

SEQ ID NO: 61 - DNA, 906 bp, Artificial sequence
  Viral double enhancer
aagccatcgaaaccggtccactggtaaaccttcaagatgaagaaagaaagattctttgccaaaaaatgcaaaagtacaaagggcacagctgtcagttgtgcaaatccgagtcatctggaccacaaacatcagaagagggtctacaagagtcagaagacgaagacttttcggtgctagtttaattactggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatctcgagcaacggagtggtcgtacatactacaaatggattgccaatagtattcgcaatggatacacaccgaggttgttgcgagaaattttgtatcacggtacaattaccagggggccttacgcgatatgattttattggcgccacgattacgaaggtaagatttggtaaagaaaaacgcaaatgcgatattaatttttcggaattaattatagaaacttcggtaggaaatatcgttttactggcagaaaacattcataatggatattactctcatgatgtattcgcttgttttgaaggtaaagttgaaacttttcgtttgtaaatacaaaaaatgtatatgagtatttgttgtcggaatgtcatatcaacaatgttgtgtatatatgtgtaaactaaaatacactata

SEQ ID NO: 62 - DNA, 668 bp, Artificial sequence
  Viral double enhancer
atcatctggagccaacaagctaggaagccgtctgagcaagatattcagatccttacagagtttttacttcggacagtcaaaaatgagtttaacttctcagccgaggtaaaacaagaaatatgcttacgtctacagagggatttctctgaagatcatgtttgccagctatgcgaacaatcatcgggagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcatctcgagaccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccg

SEQ ID NO: 63 - DNA, 793 bp, Artificial sequence
  Viral double enhancer
atcatctggagccaacaagctaggaagccgtctgagcaagatattcagatccttacagagtttttacttcggacagtcaaaaatgagtttaacttctcagccgaggtaaaacaagaaatatgcttacgtctacagagggatttctctgaagatcatgtttgccagctatgcgaacaatcatcgggagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcatctcgagaaaattatccattccagaaggtaattatccaagatgtagcatcaagaatccaatgtttacgggaaaaactatggaagtattatgttagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgag

SEQ ID NO: 64 - DNA, 778 bp, Artificial sequence
  Viral double enhancer
atcatctggagccaacaagctaggaagccgtctgagcaagatattcagatccttacagagtttttacttcggacagtcaaaaatgagtttaacttctcagccgaggtaaaacaagaaatatgcttacgtctacagagggatttctctgaagatcatgtttgccagctatgcgaacaatcatcgggagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcatctcgagcaacggagtggtcgtacatactacaaatggattgccaatagtattcgcaatggatacacaccgaggttgttgcgagaaattttgtatcacggtacaattaccagggggccttacgcgatatgattttattggcgccacgattacgaaggtaagatttggtaaagaaaaacgcaaatgcgatattaatttttcggaattaattatagaaacttcggtaggaaatatcgttttactggcagaaaacattcataatggatattactctcatgatgtattcgcttgttttgaaggtaaagttgaaacttttcgtttgtaaatacaaaaaatgtatatgagtatttgttgtcggaatgtcatatcaacaatgttgtgtatatatgtgtaaactaaaatacactata

SEQ ID NO: 65 - DNA, 728 bp, Artificial sequence
  Viral double enhancer
gagcgcttgacggtgttgaattaatttgcaggtatagttcaggaagcttcaaacaagcagaaaagaattatcacagcaatgataaagaattactagcagtcaagcaagtaatcaccaagttttcagcttacctaacaccagtcaggtttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtactcgagatcatctggagccaacaagctaggaagccgtctgagcaagatattcagatccttacagagtttttacttcggacagtcaaaaatgagtttaacttctcagccgaggtaaaacaagaaatatgcttacgtctacagagggatttctctgaagatcatgtttgccagctatgcgaacaatcatcgggagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcat

SEQ ID NO: 66 - DNA, 1603 bp, Artificial sequence
  Plant viral combination double enhancer
cggtgagggattgacgcataacagaaataactagtacctttctataaggcaaaattgagcacagtgattcactgattgtgtcccctagcttctctttccccacctacaggttgcaacgcaaaagcaagcaggaaaaaaagaaacacatttagcagtctagactctaacatcatccaacgattcatggcctgttggcatgttgctggaccgtcactggttcacaggcctttagaggatacttggccagagcgggcgcgcgtgcgcggcggcggagtgctgtggtatccagtgcgtgtgcgccgatgtggaggccaacgcagcgggcctgggctttgaggccgggatggggagcctcagggagtgcggcgacggctgcgcatctgcgccgtcatgtgggaaccgacggactcagcgcggcgtcgctgtgcggctgtgcgtcttgcgccacctgcacaaggtggtttgtgtgcgagtacgccggtaaagcagggcatatgcctctctctctggattctttgcgcttgcatactaattgttggtttgcattttgagctgaaccaatctattaaaatcgctggacgactattaaaatcgctggacgacgtagttctgtacttctgttccatgacggacgaaatgattccggatgaagctgtgcgcccgtgctgcattccaaactgcaaaaattgctgattgctggacgcggctccgtccgacggcgtacaaactgattctatatggaccgatgaggcgatccaaacagactactaaactcctacttaccaagcaaaacaattgatcaatgattacccgagtgccaaacgggcactatgaacacagaacccataacataataagctaaactgtactaatgcacaagatgcattaaaaaatgccgttttagtagattgttaaccatgtttgtgctctccacatgtcatagatatattcttataatttgttcacctgtgtttccggttagttgttcatctatgcctagctttgctttggttctgttcttgtcatagctgaaaaaagaagcgaattcattgggcaaagccttatatatagttgatacataaggattcatgtaaaatacatttgcagtccggtacagtttttactttttagccttgtgccctttgcaaaaaggactacaagaatcatgccttttttcagcgctcgagaaaattatccattccagaaggtaattatccaagatgtagcatcaagaatccaatgtttacgggaaaaactatggaagtattatgttagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgag

SEQ ID NO: 67 - DNA, 1603 bp, Artificial sequence
  Plant viral combination double enhancer
aaaattatccattccagaaggtaattatccaagatgtagcatcaagaatccaatgtttacgggaaaaactatggaagtattatgttagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagctcgagcggtgagggattgacgcataacagaaataactagtacctttctataaggcaaaattgagcacagtgattcactgattgtgtcccctagcttctctttccccacctacaggttgcaacgcaaaagcaagcaggaaaaaaagaaacacatttagcagtctagactctaacatcatccaacgattcatggcctgttggcatgttgctggaccgtcactggttcacaggcctttagaggatacttggccagagcgggcgcgcgtgcgcggcggcggagtgctgtggtatccagtgcgtgtgcgccgatgtggaggccaacgcagcgggcctgggctttgaggccgggatggggagcctcagggagtgcggcgacggctgcgcatctgcgccgtcatgtgggaaccgacggactcagcgcggcgtcgctgtgcggctgtgcgtcttgcgccacctgcacaaggtggtttgtgtgcgagtacgccggtaaagcagggcatatgcctctctctctggattctttgcgcttgcatactaattgttggtttgcattttgagctgaaccaatctattaaaatcgctggacgactattaaaatcgctggacgacgtagttctgtacttctgttccatgacggacgaaatgattccggatgaagctgtgcgcccgtgctgcattccaaactgcaaaaattgctgattgctggacgcggctccgtccgacggcgtacaaactgattctatatggaccgatgaggcgatccaaacagactactaaactcctacttaccaagcaaaacaattgatcaatgattacccgagtgccaaacgggcactatgaacacagaacccataacataataagctaaactgtactaatgcacaagatgcattaaaaaatgccgttttagtagattgttaaccatgtttgtgctctccacatgtcatagatatattcttataatttgttcacctgtgtttccggttagttgttcatctatgcctagctttgctttggttctgttcttgtcatagctgaaaaaagaagcgaattcattgggcaaagccttatatatagttgatacataaggattcatgtaaaatacatttgcagtccggtacagtttttactttttagccttgtgccctttgcaaaaaggactacaagaatcatgccttttttcagcg

SEQ ID NO: 68 - DNA, 1568 bp, Artificial sequence
  Plant viral combination double enhancer
cggtgagggattgacgcataacagaaataactagtacctttctataaggcaaaattgagcacagtgattcactgattgtgtcccctagcttctctttccccacctacaggttgcaacgcaaaagcaagcaggaaaaaaagaaacacatttagcagtctagactctaacatcatccaacgattcatggcctgttggcatgttgctggaccgtcactggttcacaggcctttagaggatacttggccagagcgggcgcgcgtgcgcggcggcggagtgctgtggtatccagtgcgtgtgcgccgatgtggaggccaacgcagcgggcctgggctttgaggccgggatggggagcctcagggagtgcggcgacggctgcgcatctgcgccgtcatgtgggaaccgacggactcagcgcggcgtcgctgtgcggctgtgcgtcttgcgccacctgcacaaggtggtttgtgtgcgagtacgccggtaaagcagggcatatgcctctctctctggattctttgcgcttgcatactaattgttggtttgcattttgagctgaaccaatctattaaaatcgctggacgactattaaaatcgctggacgacgtagttctgtacttctgttccatgacggacgaaatgattccggatgaagctgtgcgcccgtgctgcattccaaactgcaaaaattgctgattgctggacgcggctccgtccgacggcgtacaaactgattctatatggaccgatgaggcgatccaaacagactactaaactcctacttaccaagcaaaacaattgatcaatgattacccgagtgccaaacgggcactatgaacacagaacccataacataataagctaaactgtactaatgcacaagatgcattaaaaaatgccgttttagtagattgttaaccatgtttgtgctctccacatgtcatagatatattcttataatttgttcacctgtgtttccggttagttgttcatctatgcctagctttgctttggttctgttcttgtcatagctgaaaaaagaagcgaattcattgggcaaagccttatatatagttgatacataaggattcatgtaaaatacatttgcagtccggtacagtttttactttttagccttgtgccctttgcaaaaaggactacaagaatcatgccttttttcagcgctcgagggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccaca

SEQ ID NO: 69 - DNA, 1302 bp, Artificial sequence
  Plant viral combination double enhancer
gttcaaagcttgcgtgcagatactcccgtaaaacacgcatccgctcgtgtgcagttcaatcacaagtttgtggatggttggaacgcagtcacccgtcaccccacggccccacgtggccgtgacacggtcggttcaaagtccacggtgtaccacttccacccgtccccaccccaccatccttctctacttctctcctctctctcccaacggacccgatctcgtacctcacttccgtcccgcccagcttcgcttcacggcgcgccgaccggcttggacgcgccgaccggctcgtgatcagctcggtcggatccgacggtcgctcgctgctgcagcgggcgcttgctgtgcgtcgcgtgggctcggccggagcccggagggaggcggagccttccccgggtctcgaagatggcggcgccgccggcgagggcccgggccgactacgactacctcatcaagctgctcctcatcggcgatagcggtgagtgccttcaccatcacgcctcctccccctctcgctctccgtccggatcgattggctgcggtgagatctgagtgcgaaatcgaagttcggttctgattcgtccggatcgactgtgaaacgcggatgctgctgggccctgctcctcccaaaagctgccgctgtttgcctgtttcgtcgatgacgtgcaacggttttatgtatataggaatgatatgctgaaaactcacccaccatcgggtgatgactactttgctagttgatgcttgagagctacatttacaaccacacagacaagtgaaccgaaattccatttggaaaccgggatgcgacaacggacgctaattacaaaataaacagcctttgccgtcttatctaactgttacattggaatggtgattgtcggtgctcacagaaaaggaaatgcatggattttgctcgagggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccaca

SEQ ID NO: 70 - DNA, 1302 bp, Artificial sequence
  Plant viral combination double enhancer
ggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccacactcgaggttcaaagcttgcgtgcagatactcccgtaaaacacgcatccgctcgtgtgcagttcaatcacaagtttgtggatggttggaacgcagtcacccgtcaccccacggccccacgtggccgtgacacggtcggttcaaagtccacggtgtaccacttccacccgtccccaccccaccatccttctctacttctctcctctctctcccaacggacccgatctcgtacctcacttccgtcccgcccagcttcgcttcacggcgcgccgaccggcttggacgcgccgaccggctcgtgatcagctcggtcggatccgacggtcgctcgctgctgcagcgggcgcttgctgtgcgtcgcgtgggctcggccggagcccggagggaggcggagccttccccgggtctcgaagatggcggcgccgccggcgagggcccgggccgactacgactacctcatcaagctgctcctcatcggcgatagcggtgagtgccttcaccatcacgcctcctccccctctcgctctccgtccggatcgattggctgcggtgagatctgagtgcgaaatcgaagttcggttctgattcgtccggatcgactgtgaaacgcggatgctgctgggccctgctcctcccaaaagctgccgctgtttgcctgtttcgtcgatgacgtgcaacggttttatgtatataggaatgatatgctgaaaactcacccaccatcgggtgatgactactttgctagttgatgcttgagagctacatttacaaccacacagacaagtgaaccgaaattccatttggaaaccgggatgcgacaacggacgctaattacaaaataaacagcctttgccgtcttatctaactgttacattggaatggtgattgtcggtgctcacagaaaaggaaatgcatggattttg

SEQ ID NO: 71 - DNA, 781 bp, Artificial sequence
  Plant viral combination double enhancer
gaaatggctgttaacaggcacgaaatgacccgaatcgaagcctacagtcaccactcgactgccaatcacacgaccaccaatcccttcccttgtccacggcctgcacgaaaagttgtattcattgtccacggcccgtaggcgaaggcgaggcccatttagctttatgtcttgcctagccagaaaccctaaaacgccaccaatcccttcccttatccgctgtccgcctgtccggttctccctgaccctgagccgctcccgtagtcccgtcccaagtggccaggtccagctccaggagctccaggcctccagcgcgcgtcgccgccactttacgcccgtgcccagcgccccaggtagcttacgcccgtgcctagcgccgccaccttacgcccgtgcccagcgctctaggcccctaggtagcttacgctccaggcctccaggctccagacctccagtgcccaggatacatatttgattatatgtttccctcgagaccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccg

SEQ ID NO: 72 - DNA, 781 bp, Artificial sequence
  Plant viral combination double enhancer
accaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgctcgaggaaatggctgttaacaggcacgaaatgacccgaatcgaagcctacagtcaccactcgactgccaatcacacgaccaccaatcccttcccttgtccacggcctgcacgaaaagttgtattcattgtccacggcccgtaggcgaaggcgaggcccatttagctttatgtcttgcctagccagaaaccctaaaacgccaccaatcccttcccttatccgctgtccgcctgtccggttctccctgaccctgagccgctcccgtagtcccgtcccaagtggccaggtccagctccaggagctccaggcctccagcgcgcgtcgccgccactttacgcccgtgcccagcgccccaggtagcttacgcccgtgcctagcgccgccaccttacgcccgtgcccagcgctctaggcccctaggtagcttacgctccaggcctccaggctccagacctccagtgcccaggatacatatttgattatatgtttcc

SEQ ID NO: 73 - DNA, 1460 bp, Artificial sequence
  Plant viral combination double enhancer
ggtgcagcagaagcaacaagccccaaagtggatagcggccaaagcaaccagcggaagcttcttaattacggctaggcttcttaattaagtgcccatcaaatcaagctgccaacgcaggcagccagccactctcgagaagcgtatccggggcccacaaggtggaatcaacgttcaccctgccgtgacgccttgtccccagctaccccgccgtctgcctaccccattcaccgcacctccatggcccacgcgtcagtggtcctgtgactgggtgggcaggtgagcgtgagtccgtgagaaggaagcaagccctgttccgccgttcgctcgtcccatccgataactttaccgtgtgtactgtgattgtcgctctgacccgcggcacccgtgctgaagcctcccacgaccctcgtggcacggtgggacatagcgacaggcgcctcacgctcctcccccctcccctctcttcccccctgccaggcaagagagaagaaacgcacgcgagccgcgagcagaatacagcagcacgggcgccagcgccatcctgatctcgctatctcccttctcttctcccccgatgcgatctcgccaaaccctaatcccgccgcccgccgcgcggccgtcgccggagccctagccccgaatgtggcggtgacgcccgtgctgctgtattctgctcgcactggtcctgctcgtggagcacggcatcggtgagcggggccagcgccgcgggcggtgcgcgcacggagaagtccgccggaggctgccgaggcagcgtcaatgagggagatgagcgcgggcttggccttgaacgccgcgacgcggaggagcttgtagaggcacgggaggagcccccggaggtacacggcgaggtcgtcggtgggctccgaggcctcgacggcggcggcggcagcgagcgcggcggccagctgcgcgcacttgtcctgctcgtggagcacgacgtcggtgagcggggccagcgccgcgggcggtgcgcgcacggaggagggacgcagcgcagcgcggcggctaggagcctggtcacgagcggcgcgacggcatcccgcgggtgctccccagcgacgagcgccagcagccgaagcgagtgatgtcggatgcggaggtggttggaagcgcggcggggtttgcgggaggacgtgcgggcactcgagaccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccg

SEQ ID NO: 74 - DNA, 1091 bp, Artificial sequence
  Plant bacterial combination double enhancer
gtaagcctacattcagatctaaaaaccttcactctaggagttggatgtggataaagctacgaaccatacttgtaaccatctcactaaccaacccaaaacttctgtacctgcatcgtatatgtgtttatcgtgaatgtatctacttgtatttatatatcaataaaactgaatcaaaatcagcagcacgtcgttgaacaaacaaggacaaagacacactaaactcatccatcaattcttccactcatccatcttcttggaacacaatcaaggtagctaacaagctccggagctccaatccgtgccttgcctgaacggagctgaaccgacctcatcctagtcggtcatgtatcgcaagttgcaaggcaaggtcataaacctagcaaaaataaacagaacaggcccgggctggaagcagccgagctggacgcccggacccctcgatggaaaaagaagagaaggggaaaaaggggtaagaagctgggcctcgattacccaccccgaagcgcttcggatcggtcggcggtctcttccccatcgatcacgcgcgcggccgcggcctgcccgaaccgacgccgagcgacacgccgggcgatgcggtcagcaacccggccctacactgcaaaggaacgcgagcgtcagaacgtcctgaggcagcggcggggtcgggggcggaggtggaggaaggggatccggcgaaccctcgaggtcgcggcggtggacggcgacgcacgccaagcgggccgttactgggttggcgacggagaggggaacggacagaaaagggtgttggccggacaccctttttatccagcgtggcatgggcgcttttccacgtggacgtacgacgctgcgatagacacctggcagataatatctgacgggcggatggacaggacgggcgggccgagcgctggcgccagcaggccgcagtacctggaggctggggccccctccgtggtcaggcacgccgtcgcctgtcggagtcggactatcctcgagtcaagagcggagaattaagggagtcacgttttgacccccgccgatgacgcgggacaagccgttttacgtttggaactgacagaaccgcaacg

SEQ ID NO: 75 - DNA, 1593 bp, Artificial sequence
  Plant viral combination double enhancer
gcctgggccgccactgctcattttcggcccacttcattaacccgccgcgtggcccgattacccccacgcccgcgcgcgcagtcaccctaccgggtccttcgctgaccagcggggcccaatcgtcatcttcttcccccacttactcgacggctcgacgcagacccaatctcaacagaaatcgccgcatgactcggatcccgctactgcttccctggccgtgcggctgtagcgctactgacgggcaggcccgctgaccaaccttcccatcgcgcacggacttctttcgcacgcctagcgagtggagcgcggattggtcatcgggtgagatccgctgcaagctcgtcttcatcctctcgtgattaccgtggactccgcgcaacagaaatccaagcatcgcggcctagcaaatcccatcagagttcgattccaacctgcatataattgtgccctcatactaccgcacccagatgcataggacgttcgggtcttcgcgtgcgccagggagacatctaccgccatgaaacagagagaccgaaggaggaagaattgttgttcctgtttcttgggattcttctactattcaggcgttcatccctctttctctcgtggcgacgaagacgaccgacgccgacgccctgcgcctggtgccaggttccgtcgggtcttcgccggcgatggacatccaccatggccgactggcgagagagagaaaggcaacaagagcgagagaacgggtgtgttgccatcgggagggagttcaacggagccgtgcactggacgtccaagacttccctgtgcgtgcggtgtagagcagacctggcctcctcgccgggcgtgaagactgcgcgaacgccgtcgagtcgtggacctgggcgtgcttcaaagagcgccgcggtttggccttgccatcttgatcgaccggtaaaattctttccaccctgttcgcattaacaccccctccgcgtagcactcttcagatccgggttttgggaaccagagagggaaactatgcatctccggcgaacttcattgtcgtgagcttggcagcgccgccgcgcgtgggctgcttgatggggttgtagctggagaaaggaacagtggctgtcggataggcgatggatggacgagattatggcatcgtataccccttcgcgcgatcggatttgggccgtagatgcggaatcgggccgttctgatttggttagggcgtggcaagtcatcgaccgttgatcccatatccaatggacctagttgcatacagattcgttatatttgagatctaaccctagcccttgatttttaatccaacggctcagaactacagataccctttcggcctggaagacttgcataagagccactctgcttttctagaataaacccgcagtccacgctcgagagctgcttgtggggaccagacaaaaaaggaatggtgcagaattgttaggcgcacctaccaaaagcaactttgcctttattgcaaagataaagcagattcctctagtacaagtggggaacaaaataacgtggaaaagagctgtcctgacagcccactcactattgcgtttgacgaacgcagtgacgaccacaaaa

SEQ ID NO: 76 - DNA, 1598 bp, Artificial sequence
  Plant viral combination double enhancer
gaacctaaggttggaccagcacgcaacgctatctgcaggatcaaagatcgacaggctagattttaaacaccaagatccactccaatccgtacgatctgagatctatatttgatcccttcgaagcggtcctaattatctaatcaagaccgatcagatcgagatcgacgactcctactctctctcccttattcccctcaatgccttcggaccgaggccaccatggaggcgccatggccgagaccggactatgagctccctcaatgaatccccttcaaattgaaacttagctacacattgctgagactaaggcgagtagttctagggctctcttacctggagttgggacgacgaggcaccccacccacgtgttctgcggttcggcactggaaggcatgctccggcgacgaattcctctatgcccgaccaccattcgccttaacgcacgctgctccccacttcggtgccctccgtggaacgattctgcgcgcacaccgagggcctgacgctgccatacgtgaatctatcatagcgactgcttctctatgctctttctctgtttctactatggcgccgggtgggtttgctctcggctctaggggatgatggggggttgctcggcgtctggttttatagaccctagcgtgggcaagaccgaaagactcggggtcggggtcggggttacccattttctggtgaagtttgttgtggcatctgtgcaatcttgtctaggaagaagactctggacagctgggcccgtttatcagggttaagggaacgtggcgcggagcggctgattaggctgacgacccggccccagtcgtcagcggcaaaagacgttcgggctgaggaaagccagagctgacgaggcggggcccgcgcgccatacacactggaaaaagaaaaaggagggaaacgtaatgggccagcgaggcatatttggcccatcaggacagggtggacgccgagagggagttgggtcgaggagaggtttcaagcccaaaacgaggtatgtcctctctttccaatttgtttagatttcttttagtgttctaaattctaacttcaagttttgatttaatctgccgttaaaattttgaactcaatttaaatgcgcaaccaaaaatacccagcatgatgcagcaatattcatatattttgcattattttaataatttactcgagaaaattatccattccagaaggtaattatccaagatgtagcatcaagaatccaatgtttacgggaaaaactatggaagtattatgttagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgag

SEQ ID NO: 77 - DNA, 1320 bp, Artificial sequence
  Plant viral combination double enhancer
ccctttgtcgcgtgcggcccattcctcactcgccctgcctcaccgacgtgtgggtcctagcgctcctcctctcttggtgtctatggcatgtgggccgtctggccagaccgtttcaacacgcaagggcgcaagcctgtcgcgagtcttgtacacgctgacatgtggcacccggtcgtcatcactaacccctcaatcgaagacaccacaacagaatagcggcgccaggtttctcggtcgggccgctccaaccgccgttgcccgggtgtgcgggtcagactccatgctataaagagctgagccccgtacccctggaacccttggaccatctctcgccacccagaacaaggtaacctcgtcgccatcaccatagttgtggagcaccgccgcagttggcgaggtgagatcccgcggggcctcttcaatccgagtctgggggtggcaggctccctagcgcaaccaagtgggttcggttgtggggaccggtggcacgcggagtacctcagagtggcggcaattgctcgccgatgcatgccctctggtgcggactacgcctcttcgtgggcagcgaactccacatctctgacttaggtgagacccctctccgcatgttcgccatcttcccctctaggtttagcaccggacaattttaggtttggggtgtttgggcatcggattgcgtgtccgccggcgaagctccatcgcggcctcgcgggtatgccatgcggcgctgtcgtgttcttctgggatgggagatatggtcacgtcagtcgattgctcgcttaacggctgcaattagatcaaatgtaccccttcgcgcataacgatccgggttgccgattcttgatcaggcggtcaaaatcaaatccggatatcggtttgggtaggtgtaacataagtcattcgattcagatcactcgagcaacggagtggtcgtacatactacaaatggattgccaatagtattcgcaatggatacacaccgaggttgttgcgagaaattttgtatcacggtacaattaccagggggccttacgcgatatgattttattggcgccacgattacgaaggtaagatttggtaaagaaaaacgcaaatgcgatattaatttttcggaattaattatagaaacttcggtaggaaatatcgttttactggcagaaaacattcataatggatattactctcatgatgtattcgcttgttttgaaggtaaagttgaaacttttcgtttgtaaatacaaaaaatgtatatgagtatttgttgtcggaatgtcatatcaacaatgttgtgtatatatgtgtaaactaaaatacactata

SEQ ID NO: 78 - DNA, 1836 bp, Artificial sequence
  Plant viral combination double enhancer
cgctagatgggcatgttgatccttgccaaaacgctcacacagtaggtgacaaaacacataccatgacatgtacactaatttttgtttaacagattgcaaccaacaggcagctgcacctaaaaaactcattgtagcaactttgatccacaccctatgactcattccatacatggcaaaataatcttcataatgtttctgccaaagttttgggttctcgccatcgaaactaggaaagttaaggagtggtaattttcccaactccaactctaaccttgcccttggtggcctaggatcatgatactcatgtggatccagatgagaaagtggagggtgaaacatgaaatcgggcgtaccattgatcgggagatggttttgggcaaggagatgcccaaaaccatgcccccagcgatttttgtcgaagcaattcccatctgggctgtcggtgttgtacgcggcagcaggcgagcgctccgccaccgactggtacgggccgagaatacccgcggcggtatcgagctgtggcggcggcgcttgggcagcccacttggacacctcgtcacgcagaagatgcacggaggtgtggatctcgtcgaccgaagcctccacgtcgggccgccacgactcgaaggccgcgtaagccgacacgagggcaccgaggcgcattacgaaagcggcctcgacttcatcgaggcgctcgatcgctgcggtgacctgggcgtcgagttccacgtggatcgatgtcagactctcgatggcgatgttggcttccagatgttgcggtgacgcggtgttccacgcgacggttgtctcgagcgtgcaccaacgcgtctcctgagcaaagaggtggtcacagatgtcgcgaagaagcttggattgttcctcgatcgccatcttcaggttgggctccatggatccggattccttcgcgcgacgcgtgtagtaggtgtgtgctagcgcttagatcagtgtctccgatatcaatttgtgagcgtccgctccaagcaacataatctaggaatgacgagagtaggagaagggagattctagaggaagaaggagatctgatagggaggggaagtagttgttcgatatatttctctgcctaaaatcgtcacagtgcctgttcacagtatttaagtatcaactattacaaggctcgacggcccattatgccacccagtcctctcctaggaacttggcattgggcttcctgatgcggcggcccaactccagcccagccacgtccgacgacgacgcctgcttcttgacgcacgtcacctgggcgatgctgagagtgctggttacctgtaatcggatacaacgtaatcaatcggatacatgacatatatttttcttagtgctcctccggctccgtgatcggatcacgctgctgcatcttataagctgaaagaaactgggaaggtgatgagttggaccttgtgctgcccgctcgagggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccaca

SEQ ID NO: 79 - DNA, 1054 bp, Artificial sequence
  Plant viral combination double enhancer
cggcgcttgggccaaatctttcccccctcgacctaggtagaatcacctgcaccggcccatctccgtggtctccacggctcaaactcccgctgtgctggtgcctttggtgactgccagggcagacccactattcagttccacctctgcgccaatgactgatgggtctgcctcatcatccgcggttccccatccacgtgctcctgctatcaccgtcgtgtgggccatgtttgtcagtattctcctcaagctcaggatcttaaccaatcccgcgctgctcactgatatggttgggaggcaggtgctgggatccctgacctcggtgcattgggcgctcaccttgtccctataaattgtcgtgaccgtccctcgcattgaaaacccaagccgaagtaccgacaccgtgattacagagattgcacaaggaagagcagggtgaagtccgccattggaatctgccgtcgtcaactatgcatcatgggcatcaccgtcgaaccatcgggcaccagccggggagcttcgccatggcgcgaggaacgttttcgtggccccctcgagaagccatcgaaaccggtccactggtaaaccttcaagatgaagaaagaaagattctttgccaaaaaatgcaaaagtacaaagggcacagctgtcagttgtgcaaatccgagtcatctggaccacaaacatcagaagagggtctacaagagtcagaagacgaagacttttcggtgctagtttaattactggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaat

SEQ ID NO: 80 - DNA, 1482 bp, Artificial sequence
  Plant viral combination double enhancer
tgaccagcattgatacacctgatatgtcaatcgcctttaacgaaacaagaataagaagcctctcgggggtagcaataagaaactccggctcacaagttttcagtccagagatctagtgttcaatggatgcgccaggatgcaagctcactgaatgaatcccaagaggcttcaggggtttgcatatcgatcgcacttgcgcggccttatcctgtgacgagacgaggtacagcaggaaggggcaagacacgaccatcccatgcctctctttcctagcgacgatgtcgcaggcagtggagacgagccacgcggtctgcgtcaccgtggcgcacgcgccgctagcgtcgagcacgtcggacggtgcctgggtggagcaacacctccagagctccatgccccggtcaccggcgccatggatgctgccgccgcctttctcgggcgccaccgcatcccggatagcgttgaggcagaccaccaggaacttggacagccggtcgaagtccccgtggccctcatccacgcccgtcgcccttcgccatctcggctgtgctggctaggtcgcttcggcgagatagccgccaaccgtcggggagtggaggggtgggaggtcgacaggagcacgagcagttgggcggttcttggcggaatgggagcgcacatcacgaagagtgcctaaagaagatgcacagttgcttagggtggccggtgggcccggcggcgctagggcaggcacaacgctcgcccatgcgggagcgaggcacgatggccagacgcgttctcgcgagcgcactgggtgggtcgcggttgctgggccagggagggggcgctggcctggcggcctgggcgtgggcgcctgtgcgctgggccacgcggggtgggccggccacgcacgctgtaggagcgagccagggacgcggggcagctgggccgttggcgcgcgcagcggcccgggagcgcgagcaggatgcggggggaaggcgaaggcgcgtgggccggggtggccgagcggcccaggaaggggggggaacgggccacgattggggaaaatgaaggggaaagaattctgggctttttcttttatgttcctcttcttttctttgtccatttcctcgagatcatctggagccaacaagctaggaagccgtctgagcaagatattcagatccttacagagtttttacttcggacagtcaaaaatgagtttaacttctcagccgaggtaaaacaagaaatatgcttacgtctacagagggatttctctgaagatcatgtttgccagctatgcgaacaatcatcgggagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcat

SEQ ID NO: 81 - DNA, 493 bp, Artificial sequence
  Viral double enhancer
agctgcttgtggggaccagacaaaaaaggaatggtgcagaattgttaggcgcacctaccaaaagcaactttgcctttattgcaaagataaagcagattcctctagtacaagtggggaacaaaataacgtggaaaagagctgtcctgacagcccactcactattgcgtttgacgaacgcagtgacgaccacaaaactcgagacttttcaacaaagggtattatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggttgacgaacaatcccactatccttc

SEQ ID NO: 82 - DNA, 1090 bp, Brachypodium distachyon
gtacgcccgtttcccgtcgatcctcctccttccgttcgtgttctgtagccgatcgattcgattcccttacacccgttcgtgttctctcgtggatcgatcgattgtttgttgctagaaggaactcgtagatctggcgtttatgaactgtgattcgggttagtccagatcgattcaggtcggtcgtcgttgagcctctcggctatgtctggattatcgtgtagatctgctggttcagttgattatgttcttctaggagtaatttcgttgggtcagcgcgatttctgcttaatctatgctgcttattgcgcctgtacctatctactaagctatgtgcacctgtaattttgctagattattcgttcatcctcgtagttggtttgtcacagtaatccgtatgggttctgacgatgttattgttggtcatacctacgcttctccagattttattttgttaaaattggatagatctgctactgatagttgatgatggaatttggtgctgaatctatgctatttattgcgcctatacctgatctatcgggctatgtacggctgtagtttactggattattcgttcatcctcggtagttggttcatcgtttgggttctgacgataatattgttgattatgcgtaggcttctgcagattgttgttaaaattggatacatcggttactgatggttgatgatagatttgtgctgaacctatctgtttattgctcctatacctgatctatagggctatgtatgcctgtaatttaccagattattcgttcatcctcgtagttggttcatctctataattcgtatgggttcttatgatgttatcgttgattatgcctagtcttatacagattattgtgtcaagattgaatatacctgctactgatcggtgataatttggttagtagtttgcaatctgctaggaacacgttaccactgtaatctgtaaacatggtttgccagagtagtttgttctactactcttgatatggttgctgattttagtcgcctccttttggatcatgtattgatgtccttgcagatttccgtgtacttaccccggcttttgtgtacttcgtgttaacag

SEQ ID NO: 83 - DNA, 707 bp, Brachypodium distachyon
gtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccttggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcag

SEQ ID NO: 84 - DNA, 510 bp, Zea mays
gtacgccgctcgtcctccccccccccccctctctaccttctctagatcggcgttccggtccttggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 85 - DNA, 1291 bp, Panicum virgatum
gtacggcgatcctcctctcccttctcccctcgatcgattatgcgtgttccgtttccgtttccgatcgagcgaatcgatggttaggacccttgggggacccttggggtgtcgtgtggtggtctggtttgatccgcgatatttctccgttcgtagtgtagatctgatcgaatccctggtgaaatcgttgatcgtgctattcgtgtgagggttcttaggtttggagttgtggaggtagttctgatcggtttgtaggtgagattttccccatgattttgcttggctcgtttgtcttggttagattagatctgcccgcattttgttcgatatttctgatgcagatatgatgaataatttcgtccttgtatcccgcgtccgtatgtgtattaagtttgcaggtgctagttaggtttttcctactgatttgtcttatccattctgtttagcttgcaaggtttggtaatggtccggcatgtttgtctctatagattagagtagaataagattatctcaacaagctgttggcttatcaattttggatctgcatgtgtttcgcatctatatctttgcaattaagatggtagatggacatatgctcctgttgagttgatgttgtaccttttacctgaggtctgaggaacatgcatcctcctgctactttgtgcttatacagatcatcaagattatgcagctaatattcgatcagtttctagtatctacatggtaaacttgcatgcacttgctacttatttttgatatacttggatgataacatatgctgctggttgattcctacctacatgatgaacattttacaggccattagtgtctgtctgtatgtgttgttcctgtttgcttcagtctatttctgtttcattcctagtttattggttctctgctagatacttaccctgctgggcttagttatcatcttatctcgaatgcattttcatgtttatagatgaatatacactcagataggtgtagatgtatgctactgtttctctacgttgctgtaggttttacctgtggcaactgcatactcctgttgcttcgctagatatgtatgtgcttatatagattaagatatgtgtgatggttctttagtatatctgatgatcatgtatgctcttttaacttcttgctacacttggtaacatgctgtgatgctgtttgttgattctgtagcactaccaatgatgaccttatctctctttgtatatgatgtttctgtttgtttgaggcttgtgttactgctagttacttaccctgttgcctggctaatcttctgcag

SEQ ID NO: 86 - DNA, 129 bp, Zea mays
gtgcggtgcccccctccctcgctcttcctggccccgatctgatccgctctctgcgttagggtatgggtgacggggtggcactcgatctgccgggttcggatctgattttcttgttgtcggtttatgcag

SEQ ID NO: 87 - DNA, 980 bp, Zea mays
gtaaggccccctccacccctccgcttcccctcccccgggcgcgctctggcttcctccccggatcggcgcggggcgtgctggctccgcgcctgatttcgggccttttgtttccttctcgcggagcgctcgtgtaacgcttcggatctagctggattcaggcgggatcgcggccgctcggcttcctcgtggcctgattcgtggttttcctcggggagggaatcctgatcggatcatcgggattcctcgtgcggccgggacacgcttgcgagccagaaacatagtctgcgtggccgggattccacgatctgtgatctagacgtcgggcgcttcgtctatgtgctcgctgcaggctgtggcgtactggcgtggtgcgcggccgctatggatcagtgcttgtttgttcgccctgtagcgtgtgaaatcgagctgtgtagatctatggtctgcgaggtgcggtggcggtggaatctcggttgatctttacctcagcggcgccagtgtagctcgtgtggctgcagttcatctgcgaatttggctctcggcggcttaggtcgcggagcttggattatggagcaccagctgcagcgtgaccctgttggttctcatgtggatctgttggctgaggttgcagacttcaagtgccactgccattgaccggagctgctgcacgattatactggaatatctagcggtagtatactctgctagtactcaatacgggtctcctgacaaatgtctttcgtgtttagggacctagcactctagtgtcaagactatttgctggaatatctaatattagcagtttctgtagtggctcagttgcagcctggtttagaatgatggggacagttggctgtgccatgcaaaataaagtgtgtgaaagcaactgcctcttaaactatgggtggtgcaagcaggttatttgaagggactctccacactgtatctccagttaactatgactgaacttgtggtcgcag

SEQ ID NO: 88 - DNA, 714 bp, Zea mays
gtagctaatcctctgtcgctcgatctttgtccttgaggtgctcgatgagataggcagtggttgaaagctgtgctctgttgcctccccgtgcctgaagtgattcggtcttctgtttgcgattaattgtagctactgcgtctccattctcttagttttttgtggttttgtgtgcttccatgtttggatgacctgattcttacggatagttgcttctggatttgataaattacatacgcaccgtgccaccgtagttggacgacttcttaattgcgcttgcggtcgtgttacgtggtcttaataaagtcagatcgaatttttgaggtgtagagctgctgcagtttctatttgaccgatacttgtctttgtagttcatgcgcagtaagagttgtaacctcataatatgaccaaattcctgatctgctttatagagttgttgtgcctaattttgaattcgtcttactggctcaaaattatgtgcgcacgcttattagtttgccctttattttctctgcctgctttatttttttatattacattgattgctctgtttaatatttgcttctttatttttacactgcttatgcgtatcaagctcttttcaagctcaccggtgacaacgttagatctagtttctgatcccatctgataaattgtgtcgttactggtagtattaatttactagtgtttctaatcgttgattcgtcttgttaacag

SEQ ID NO: 89 - DNA, 199 bp, Zea mays
gtatgcttgctgccttgctcttcctgttgagatgaatcatatatagtatagctgcatactacaaatctgtttttcaaatttaggttgctttggcatgatctatttttttgtcagacagactttctaagtggtagctcttgatttcttgttcttgtacaactggtgctgctgaatcttgaccgtatagctcgaattgcag

SEQ ID NO: 90 - DNA, 106 bp, Zea mays
gtactgatgtctctctctctctctctctcttactctcccctcgattttagatctgcctgaaggacgaatcatagtgacctcacgttggtgcgtttttctccaccag

SEQ ID NO: 91 - DNA, 783 bp, Zea mays
gtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcag

SEQ ID NO: 92 - DNA, 1010 bp, Zea mays
gtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcag

SEQ ID NO: 93 - DNA, 999 bp, Oryza sativa
tcagccagtttggtggagctgccgatgtgcctggtcgtcccgagcctctgttcgtcaagtatttgtggtgctgatgtctacttgtgtctggtttaatggaccatcgagtccgtatgatatgttagttttatgaaacagtttcctgtgggacagcagtatgctttatgaataagttggatttgaacctaaatatgtgctcaatttgctcatttgcatctcattcctgttgatgttttatctgagttgcaagtttgaaaatgctgcatattcttattaaatcgtcatttacttttatcttaatgagctttgcaatggcctatgggatataaaagattattctggagggaagtgatgctggaaggactatgctgtcctgatttatatttggagccactatgagcatttgggctttcttttcagaacgctgtaggcgtgtgttgaaatctttgcgacattcaatttgatatatgattcgaggtaattgggctttaatttgtcatctcatgtaacatctttttgtttcttcgctgcttgatttctctatttcgtagcattggaagataatagtagaatgatgatatactccaatacttgcaatttcaaaaccgttagaaagaaaggaaaatcacggctaggggaaaaattctctctgatgcgtgcatcaccaaaatctgattgattgacataagcattggaacacaaatagaagatggagaggtggacatgtttccgagacatagcatttgagctgacattgtttttgatccgtggctgtgccatacgcgataatttactaaactttcccttgccccttggtaatataaagcttataatgtatacaccgaaattgaaaaacgtattagttcactcttcttcctttttctcagcaaagcgtgttcatcaattttattccacatgaatgtgattgcgaattgtgaccaggaatgcatgatttaacaatcccgatcaaccctgttgtcggtggtggcactaaaaatt

SEQ ID NO: 94 - DNA, 1013 bp, Zea mays
cgcatcatgatcatgcatcatggactcggcctactactgtggatttgtatgccattatagacttggtgctgtgaaagactgcttgatgatttgcgggtttgttgctgtgtaaaaaaaggtcccttggctcccagaagaccatgaaggttcggatctatcatgtaattccttgttatctgccaattatgtatggactatggacatgtgttgcgctgttcaacttactactacaaataagtaatcgatatgttcccttcccatgtctcggtgacaattgtctggagaagcttaggggtcgtttgtttgggattatgtctggagaaacttattttaaactaagtgtgagttcaagttaagttagattatataatctaggcagattataattccaagcgaacaggtccttagtgtttttggaaaatcctaggtgttcttttggctacattgttgtgtgtgcagatcccttgttggtctgtaagcgtggggaagtaagaatcgtccgtttctactgaagacctgctcgagttaggcaccgaggatgccggtaaccaaacagagcaatagtgtctctgtgggcacagtggagtgtgaatctgtgtgatgcaaatccgtcatttgtttagcaaaatttccagcgttgcatgatgcagtttctttaacacggacttaagggaagggaaaaaaatgttgagccaggagatccttcaatgtgttagactgacgtgatagccaactaaaccacgacgcaatgttgtcgttaatgacaaaaaaactatttgttcctaaatccttggcgacattgcatggctgtctcatgagataatggtctcatctcttatttatctcttatttatagccggaagtggtagtgacccctgcttgattgctcgtatgccatctcaagttctcaaccgtgtcgagcagccattttcccatctcaagcgcatcatcgtttcgtttgacctcatctgctatcctgctcctagtgcaaatcacatgcgacagaaagtgtg

SEQ ID NO: 95 - DNA, 1304 bp, Zea mays
gctcatgacgatcgatctgttgggtgatgcctactctgtccatcgtgctcgacttgtcttcctactactgtaccactgtctgtgctcactttcacgttcctgacggtgttgtgttctagtgtatcattgtgcgatcagttccatgttcttggccatgttttagaataaaacagaaaacttgtgttcttggtgtaccactgtgctcactttcatgttcttgacggcgagttggaataaaacaagacttgtgtcatggtgtaccattgccatcattttcatgttaatataaagatttgacgcaagacgcaacattcgtaaacacatatatccacatcaaataaattagggcgtgtttggtttacggtagattttaagaatctgattttaagaatatattatcgatccaaacagagtttattctgtgccttagattctacgctcatagaaacaaaatctgtagaatcaatccatagttgggttttcttgggtaacgtgaacatgcagattttaaaaatctgcggaaaccaaacgcggccttagagacatttgtgacggtagctatagatgtccaaacgggccacccggcacggacccggcccgaacccgtttcggcccgatacgaacagggccaggccaggcacgacccgttgatgcttcgggccgggtcgagctggcccacgtgcctagaaacatgcccaaacccggcacgcacagggaaaaatcgtgtcgggccggcccgttggcccggtgggcctcaagggaccgggtcgggcactggcccgaaccaacagtagaacggtatataaatacatatatttaacagaattaatcatatataataacatattatttatatatattaagacaacaaaatatttatgtatgcattttatatttatgtatgcattttatatatgttttggccgcgtatttagtatttatatactatgagagaattaaatgtatttacatatttagtatttatataaatatgcgggccgccgtcgggccgacccatttatcgggccgggccggggccacagcacgcgggccgccgtatctgtccatacccggcccgctaaacgggccgtgccggcccggacccgtaaccgaccgtgtcgggccgtgtttggaccgggctaaattcgtgtcgtgccacgggccaaacgggcggcccgcactgtttggacatctataacggtagccttctcccaccttacaattatgacattgcatcttttttttattttttaatcattaattttaattttcactaatagtgttaaaagagataggtaaacggc

SEQ ID NO: 96 - DNA, 1000 bp, Panicum virgatum
gccattggtcatccaagctgctgctgtacctgggtcgtctggtctctgccatgaagggcgtcttctttgaagtatctgtgtcgtttgtctaaactgtgtttggttttcatgaactggttgtgtccttgtgttcctgaaacagtcattgcggccgcagttggcttaagcatgaataagtaagactcaaatttgtgctctgtttatacattatgccagttctatataaattctatagcttttgagttctctttggcttgtgaggtgtttgttgcacctccagattcttgctgactcccattttgtactgtatgtccatctctgttctgccaagctcaaatttctaactgattgtttgcaagtgttggattcgatttggaggatctgacatgtttggtttgttgctcgagtgttactagtagattaattaagaagcttttcgctagttatggaatcgagtaggtctgtgctatgcagaaccttatttttggtagactcgtgacattgctctgaaccaggtaattggggatcatatttaactggaggtagacaaagtgactctctgcgtgtatgtagttttgtgtttgaccattgcaataagccaataactgaagtagatacgtttcatactcctaattcaaactgaatcgattgtcgtggtttgttgctcctacaatttgctagtttttttttaactgaaccaatgcctctgtttgctgtgttgtggccgctggctggtgtttgatttgttgtgagagaaaagtactgctggcagacagatggctgttgactggtgctggtttagtacgagaaaaacactatcggctggatgcagcgaacagagtgaatatcggagtgtgtctgttgcccacacattcatgctttctttttgtctggatgctgtgtacaaaataaatattctgcttcgccattgccaaacaggcttgcaggcgaaaatctgtctggcacctatcctagtaccctaccaataccgttccaggcc

SEQ ID NO: 97 - DNA, 980 bp, Oryza sativa
ttcttcggacccaagaatgctaagccaagaggagctgttatcgccgtcctcctgcttgtttctctctttttgttgctgtttcttcattagcgtggacaaagttttcaaccggcctatctgttatcattttcttctattcaaagactgtaatacctattgctacctgtggttctcacttgtgattttggacacatatgttcggtttattcaaatttaatcagatgcctgatgagggtaccagaaaaaatacgtgttctggttgtttttgagttgcgattattctatgaaatgaataacatcgaagttatcatcccagtattttcgcatgaatgttcttttcttctgtcttgtgcatcagtgatctagtgcatgggagtttgtattgtgatgttcgacatcacgtaacttccactttgcctttgctgttcgatattttaatgacatgtcacacacacttctgatacttttctttcttggctattgtgccagcatgatgcaagatgcatcacagcatcagatatattctcatcgtcaggctttagcagcacacgagcacgctttgccgcttaaaagttgtacggcgcagcttagacatcccctgtagaagtgataatcttttcacttttccttaaacaaattgagaggggaaatggaaccatgtggatcagagaagcttttgtttctttacacaagaatatttggtacagtgggggtcctatgttcgtgggttcgtggcttggctgcctgtcttcaaccaagtgttttcagttcaacatgttagcgtgtagaaagagcacaattctgtttatctccaaggtaaaatgtggcattctgttaaagaacatgatcctgccaattttttaagtttcaatggaagaggaatgtaaagctttctatggtttgtgtacacaacacagtggaagaggagtgcaagctttctatggtttgtgtgcgcgttgtgtgtcagcacttcaattttg

SEQ ID NO: 98 - DNA, 1035 bp, Zea mays
gtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 99 - DNA, 1000 bp, Saccharum officinarum
tcctgggccatgaagctgtccttccaggttcacaagtctggtgccttcttctgtccctccgatggagattatctgcatgtcgtggtcgtgtcctgatcgaatcctcgttgaatccctatgtttttcttcaagaaatgtgagtcctatgtcagtctggttgcgtttgtgaacatttctgctgctgagcagcactttggctggaactgtgcaatgaaataaatggaaccctggtttctggttatgtgtgtgttagctaatgtttttgaagtggaagctctaatcttctatcgcgttgctactacaattctgcttgtgttttgatgttcttggtttctgttagttggttcagaggaagttttgcttccacagactaagatgcagttgaactttggttgccctggtttctagatttcatttgtgctggttgagtgatagtaagaaacaaccggtgttcacatataatcaggttttgtgctgctcgagtgatcgtcaaaaaccaccggtgttcacatctaaaaaggtttcgatccccaggtttagatctcccgtttaattccaaaaaaaaagttctgtgtacttgcatttagttgggtggttgatgctggaaagagtaactttcaagagtaataatctttggtgactactctgtttcaactgatcaatccctaggaaaggtacacctttacttagggaagaaattcttagaaccttgcactttgtttcaactgataatagtatactttattagataaaaaatattcagatatattagacaccggatgtcatccactcatccttacaaacctctgtcatggtcctgcagaaatgtttgccagctccagtggcttcctgataaatctgtggagtgcctgttaatcggctgccaatttttgctgagcactgtatatatgttagtaagtactattgggccaccaattcgattttgacacagcactattggtccaccaattcgattctgacacagcactgcataatttga

SEQ ID NO: 100 - DNA, 999 bp, Brachypodium distachyon
tttgttctgtgttggggattgagatgtctggctgcagttttagtgtttactaagttttgtctatgtgcattgctcgtattgctgagactcttgaaactatgtaagcaatgttcatgtctgtcgtttaaatactgtattcctatttcggttcggttaaaacctgtgtgctatgatgcttgtgtccttttaacttctcagggatctgattgcttagattgaacttagttaactcatgttctgtttccttgatttcttcgcaatttgtttttgttctgctattccatctttatgtagcagcgtagcctgtaatcatgtgatacgtttatgtcctaatttttcttttttcactgttctccgtttttctgattagaggttggttaggtttctccgcttctttcgtccgcagcagattttccacttgttgctgctggcttgaagctagagtacataataatttcaatataaataattactctttgtttgtaacactgcaactaggctatcttgacgttggcctcttaactccaaagtattggtttgtattcccttttctcgcattcgctgcatacggcgatcaacttcgtagtagatcgttatattctggcctcgaagcacaatttgtcctcagagaatttgtgcacttgagttccagattctaactaaaatacgaatactgtatgttattctggccccgaagtactctttgtcctttcagaggaatcatttgggcacctgggtttccagattctaaccaaaaagttgatattctggtccccaagcactctgtcctctcacggcagaatttgtgccacttggcgttcagattctaatcgccttcttgcttgaaggaacttcccttccgactaaaataatcaaaagctctttgcccttaaaaagaagcttaattcatctcaagaattaaaaaaacctactaattcatctaaatattcaaatcgacgggaattaactccaacacgcgaggctttgacttttgaatta

SEQ ID NO: 101 - DNA, 253 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 102 - DNA, 2627 bp, Zea mays
tctgcctttctgttcttcaaacgatgtctcatgtctgcgctggacaactttcttgttgccgcctgtcgcttgcgctgtgctgactggacgcagctccggaggtttggttgtgcttggttttcgtagagaactcgccacttgccgcccgcacgttcttggtgtttcctcctccccgctgtgttctgcgcacgggctttttctgagagacccatgtttcccttttacttttataaacagtatacatgctatgtttctagaaggaggggaaacctaatccccctaatccaatggcggggaggaaatagggtggggtggggtggggggagggaaatatctcgctactttttaatccggacaagctcatttgcgtttgcgtctgaatgatgatgactgcaatgctgatcgcacctcgggtgtcggatcaccagcttttggctgctctcaccaaatcagctgcaagaagattagagcacaaaagaattacagaaagagagcctttttcttttcttccttgtggggttcctttcatttcgtgctctcctttctctgccagccagtccgtccttgcgtccactgcacctgcacacaggtcaccccgacccgcactgttctagactccattagaaaaaaaaaggtctgaacctttccgaaaccagccagccattggtctggcaggccagcatatgctaattggatttttttgccgcatcattgagtgcgccatcaggatttggaaatcctggttttgagtaatacagtaatttggcattatccattgccgaattcccaagctccgtcagcttgaacgtggacccctaccatctgcaccagctcggcacctcacgctcgcagcgctaggagcctaggagcagctgcccgtctatttattggtccctctcccgtcccagagaaaccctccctccctcctccattggactgcttgctccctgttgaccattggggtatgcttgctgccttgctctcctgttcatctccgtgctaaacctctgtcctctgggtgggtttttgctgggattttgagctaatctgctggtcccggtagaaaaagatcatgtcccctgacgtgctcaagcgctcgccttagccgcgtccttgccccccgccattttttgcggtttcggtgtgttcccgtgactcgccgggtgcgtcatcgcctgaatcttgtctgggctctgctgacatgttcttggctagttgggtttatagattcctctgatctaaaccgtgcctgtgctgcgcacagaactctcccctgtcctttcctggggttttggttacgtggtggtagtaagcttggatttgcacatggataaagttgttctaagctccgtgggttgcttgagatcttgctgttattgcgtgccgtgctcactttttttgcaatccgaggaatgaatttgtcgtttactcgttttggtggattattagcgcgaaaaaaaaactcttttttttttgttcttttactacgaaaagcatcttcttggattttgctatcttcttttactacgaaaaactcttgagtctaggaatttgaatttgtgatgtccattcttgcagtgcgctgtgctttattgggaagccaaatcctattattttctgcctctagggtctgaatggaatcagtactcttgagacagaaaatcaatccaatcaagttgatttctttctttaaaaatattatcacagaactaagtgcttgtgcggaatcagtactggcttttgtttggtggaggatcaatacttgcttttgtttgggggtggcaactgttttgctataagattccatgtgttcctgttgagatgaatcatatatagtatagctgcatactacaaatctgtttttcaaatttaggttgctttggcatgatctatttttttgtcagacagactttctaagtggtagctcttgatttcttgttcttgtacaactggtgctgctgaatcttgaccgtatagctcgaattgcagtattctgaaccatcgagccaaggctgccaagctgactcgcctccacagtcttcgcgaacgccttggtgccaccttctcctcccatcccaatgaactgatagcactcttttccaggtgggcttaccaaaatcatataacttgcatttcattcggtactgaaagttgttaatttgttattctcttcatgcctgtcttaatagcacacccagatgtaaacacgagattatgcaacttcttacttggtttcttttgttggcaccatcatgcatgctaattgctaaggatgttacctattcatccttgactcatattatcatatgtaatgattttatgatcacgagactattgattgtgaagcatagtatagctgttcttcagtttttgtacccttttgtttttttccttaagctagaactggtacaatttagttgataagacagtgtagtttgtagtacgtcatttgacagattgtttgtctttagctggtaaagtgccatttaatatctgtatccttcagatctaataaaaaggatatgagatgtccatcacaagaggggaaaaattacatgatctgagatgtaacatccgtttttatttgtgaaataccacttctacaggtatcttcactagggtaaacc

SEQ ID NO: 103 - DNA, 1947 bp, Artificial sequence
  Truncated, codon optimized for expression in maize
atggacaacaaccccaacatcaacgagtgcatcccctacaactgcctgagcaaccccgaggtggaggtgctgggcggcgagcgcatcgagaccggctacacccccatcgacatcagcctgagcctgacccagttcctgctgagcgagttcgtgcccggcgccggcttcgtgctgggcctggtggacatcatctggggcatcttcggccccagccagtgggacgccttcctggtgcagatcgagcagctgatcaaccagcgcatcgaggagttcgcccgcaaccaggccatcagccgcctggagggcctgagcaacctgtaccaaatctacgccgagagcttccgcgagtgggaggccgaccccaccaaccccgccctgcgcgaggagatgcgcatccagttcaacgacatgaacagcgccctgaccaccgccatccccctgttcgccgtgcagaactaccaggtgcccctgctgagcgtgtacgtgcaggccgccaacctgcacctgagcgtgctgcgcgacgtcagcgtgttcggccagcgctggggcttcgacgccgccaccatcaacagccgctacaacgacctgacccgcctgatcggcaactacaccgaccacgccgtgcgctggtacaacaccggcctggagcgcgtgtggggtccagacagccgcgactggatcaggtacaaccagttccgccgcgagctgaccctgaccgtgctggacatcgtgagcctgttccccaactacgacagccgcacctaccccatccgcaccgtgagccagctgacccgcgagatttacaccaaccccgtgctggagaacttcgacggcagcttccgcggcagcgcccagggcatcgagggcagcatccgcagcccccacctgatggacatcctgaacagcatcaccatctacaccgacgcccaccgcggcgagtactactggagcggccaccagatcatggccagccccgtcggcttcagcggccccgagttcaccttccccctgtacggcactatgggcaacgctgcacctcagcagcgcatcgtggcacagctgggccagggagtgtaccgcaccctgagcagcaccctgtaccgtcgacctttcaacatcggcatcaacaaccagcagctgagcgtgctggacggcaccgagttcgcctacggcaccagcagcaacctgcccagcgccgtgtaccgcaagagcggcaccgtggacagcctggacgagatcccccctcagaacaacaacgtgccacctcgacagggcttcagccaccgtctgagccacgtgagcatgttccgcagtggcttcagcaacagcagcgtgagcatcatccgtgcacctatgttcagctggattcaccgcagtgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtag

SEQ ID NO: 104 - protein, 648 aa, Artificial sequence
  Truncated protein sequence
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQV

SEQ ID NO: 105 - DNA, 1627 bp, Oryza sativa
ggaagctaactagtcacggcgaatacatgacgacatcggcctacaacgcacaacttcttggcataaaagcttcaatttcaatgcccctatctggaagccgtaggcgccgcgcaaatgtaaaacattcgcttcgcttggcttgttatccaaaatagagtatggacctccgacagattggcaacccgtgggtaatcgaaaatggctccatctgcccctttgtcgaaggaatcaggaaacggccctcacctcctggcggagtgtagatatgtgaaagaatctaggcgacacttgcagactggacaacatgtgaacaaataagaccaacgttatggcaacaagcctcgacgctactcaagtggtgggaggccaccgcatgttccaacgaagcgccaaagaaagccttgcagactctaatgctattagtcggctaggatatttggaatgaaaggaaccgcagagtttttcagcaccaagagcttccggtggctagtctgatagccaaaattaaggaggatgccaaaacatgggtcttggcgggcgcgaaacaccttgataggtggcttaccttttaacatgttcgggccaaaggccttgagacggtaaagttttctatttgcgcttgcgcatgtacaattttattcctctattcaatgaaattggtggctcactggttcattaataaaaaaagaatctagcctgttcgggaagaagaggattttgttcgtgagagagagagagagagagagagagagagagagagagaaggaggaggaggattttcaggcttcgcattgcccaacctctgcttctgttggcccaagaagaatcccaggcgcccttgggctggcagtttaccacggacctacctagcctaccttagctatctaagcgggccgacctagtagctacgtgcctagtgtagattaaagttgccgggccagcaggaagccacgatgcaatggcatcttcccctgtccttcgcgtacgtgaaaacaaacccaggtaagcttagaatcttcttgcccgttggactgggacacccaccaatcccaccatgccccgatattcctccggtctcggttcatgtgatgtcctctcttgtgtgatcacggagcaagcattcttaaacggcaaaagaaaatcaccaacttgctcacgcagtcacgctgcaccgcgcgaagcgacgcccgataggccaagatcgcgagataaaataacaaccaatgatcataaggaaacaagcccgcgatgtgtcgtgtgcagcaatcttggtcatttgcgggatcgagtgcttcacagctaaccaaatattcggccgatgatttaacacattatcagcgtagatgtacgtacgatttgttaattaatctacgagccttgctagggcaggtgttctgccagccaatccagatcgccctcgtatgcacgctcacatgatggcagggcagggttcacatcagctctaacggtcgattaattaatcccggggctcgactataaatacctccctaatcccatgatcaaaaccatctcaagcagcctaatcatctccagctgatcaagagcacttaattagctagctagtgattagctgcgcttgtgatcgatcgatctcgggtacgtagca

SEQ ID NO: 106 - DNA, 1000 bp, Oryza sativa
gcagcgtgcatatgcaatgcctgcatgggttgatcctacgtacggtgattagttggctttgacgactcttgatttgatttgcttgctgctctgtttatttgctactacgttacgtacgtactttgcatgcaacgcaacgcatgatcgatcgtgcatgctggctgtttgtacgtatcaccgtaccagtttggattctctctgtactctctcctttgtcttctttgtagtactcttattcccgctatccgtacgtgcgcatttgttgtaagggccggtgctagcttgtgtgcccgtaccaacttctaataaagctttggtttgcaacttcagatatatatgtacttctgtttgtgattgtttgctgtgtgtgtacatatgtgtgcgtgcacgactacagctccgcgcgtgccaaatttgtgagaagtttaacatcgttatatgatttatgcttttagttttaaaaaaatacttttataatatgtactcgtatgttgttaaactatacaaaattttagataccgtagttaaagataaaaatagttgactttaggacaaaactaaaatatataccatatatatatgaaacggagggagtaatgctaatactctagctttcggtactacatctctagaatcaaaatcgttcatactctctccatccatattataagtcatttgatttgtagtaagtcacacttgaatagtttgaaaaagctcatttaaaaaatcaataatattctaacatgaaacaaatgtactcttgaaaatatattcaaggatggatttactaaaactagttttgtattgtagatgcttctatgttttctagacttagttaaacttaaaacaaaaataatataaaacaaacatcaatgtcttataatatgtaatagagagagcccatacattcgtccaggttatgaaataacacgttcaattgttaataatgactatcgaacaactttaaaattttctatatgctttgtagttggtttcaaaa

SEQ ID NO: 107 - DNA, 1962 bp, Bacillus thuringiensis
atgactagtaacggccgccagtgtgctggtattcgcccttatgacggccgacaacaacaccgaggcctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtag

SEQ ID NO: 108 - DNA, 23 bp, Zea mays
gcgagaggggcgtgggacccacg

SEQ ID NO: 109 - DNA, 107 bp, Zea mays
  misc_feature (51)..(57): n is a, c, g, or t
ctcccttccccccactaataaattactaacacatcgtgggtcccacgcccnnnnnnncccacgcccctctggggttacacgcacgcgccgtccttttctcccctcta

(19) United States
(12) Patent Application Publication
(10) Pub. No.: US 2024/0301440 A1
(43) Pub. Date: Sep. 12, 2024

(54) MAIZE REGULATORY ELEMENTS AND USES THEREOF

(71) Applicant: SYNGENTA CROP PROTECTION AG, Basel (CH)

(72) Inventors: Kasimalai Azhakanandam, Research Triangle Park, NC (US); Jonathan Cohn, Research Triangle Park, NC (US); Zhongying Chen, Research Triangle Park, NC (US); Qiudeng Que, Research Triangle Park, NC (US); Pei Su, Research Triangle Park, NC (US)

(73) Assignee: SYNGENTA CROP PROTECTION AG, Basel (CH)

(21) Appl. No.: 18/268,343

(22) PCT Filed: Dec. 20, 2021
(86) PCT No.: PCT/US2021/064343

§ 371 Date: Jun. 20, 2023

Related U.S. Application Data
(60) Provisional application No. 63/128,917, filed on Dec. 22, 2020.

(51) Int. Cl. C12N 15/82

(52) U.S. Cl. CPC C12N 15/8286; C12N 15/8205; C12N 15/8227

(57) ABSTRACT

Provided herein are regulatory elements, such as transcriptional enhancers, introns, and terminators derived or obtained from various sources including Zea mays and plant viruses. Such regulatory elements are useful for expression cassettes for plants, including monocots (e.g., maize) and dicots (e.g., soy). Enhancers which increase gene expression of tissue specific promoters while maintaining tissue specific expression are provided.

### RELATED APPLICATIONS

This application claims the benefit of provisional application 63/128,917, filed Dec. 22, 2020, and incorporated by reference in its entirety herein.

### FIELD OF THE INVENTION

The present invention relates to regulatory elements, e.g., transcriptional enhancers, introns, and terminators, which are useful for expression cassettes in plants, such as corn.

### SEQUENCE LISTING

This application is accompanied by a sequence listing entitled “82215-WO-REG-ORG-P-1_ST25.txt”, created Dec. 20, 2021, which is approximately 141 kilobytes in size. This sequence listing is incorporated herein by reference in its entirety. This sequence listing is submitted herewith via EFS-Web, and is in compliance with 37 C.F.R. § 1.824(a)(2)-(6) and (b).

### BACKGROUND

Genetically modified plants are designed to contain desirable traits, such as insect resistance and herbicide tolerance. In general, to create such traits, one or more nucleic acids are introduced into a plant via expression cassettes that express one or more coding sequences for one or more traits. Such expression cassettes generally contain a promoter and a terminator sequence to control expression of each coding sequence. Some expression cassettes also contain transcriptional enhancer elements that promote transcription and increase activity of nearby promoters. Similarly, the expression cassettes could contain introns which play a role in modulating the expression of trait proteins. For certain traits, such as insect resistance and herbicide tolerance, it may be desirable to use regulatory elements which would provide medium to high constitutive expression and/or medium to high tissue specific expression. However, the choices for such regulatory elements remain limited in part due to the low predictability of the effect of putative regulatory elements on gene expression. There remains a need for additional regulatory sequences that drive gene expression for robust protein production, ideally in all or most maize tissues.

### SUMMARY

The present disclosure provides novel transcriptional enhancer elements, including chimeric enhancer elements comprising various combinations of viral and plant enhancer elements that can be used to augment the transcriptional activity of an associated promoter. The enhancer elements disclosed herein may be operably linked, in expression cassettes, to promoters, terminators, introns, and other regulatory elements to drive higher levels of expression of a gene of interest.

### BRIEF DESCRIPTION OF THE FIGURES

FIG. 1 shows an example of a chimeric transcriptional enhancer operably linked to a promoter to augment the expression of a heterologous nucleic acid driven by the promoter.

FIG. 2 shows expression vector 25356 with a chimeric transcriptional enhancer comprising enhancers eZm18 and eMMV.

### BRIEF DESCRIPTION OF THE SEQUENCES IN THE SEQUENCE LISTING

SEQ ID NO: 1 is plant enhancer eZm00001d002338 (eZm1).
SEQ ID NO: 2 is plant enhancer eZm00001d004841 (eZm2).
SEQ ID NO: 3 is plant enhancer eZm00001d005179 (eZm3).
SEQ ID NO: 4 is plant enhancer eZm00001d005798 (eZm4).
SEQ ID NO: 5 is plant enhancer eZm00001d017850 (eZm5).
SEQ ID NO: 6 is plant enhancer eZm00001d020804 (eZm6).
SEQ ID NO: 7 is plant enhancer eZm00001d029426 (eZm7).
SEQ ID NO: 8 is plant enhancer eZm00001d030618 (eZm8).
SEQ ID NO: 9 is plant enhancer eZm00001d034223 (eZm9).
SEQ ID NO: 10 is plant enhancer eZm00001d034223 (eZm10).
SEQ ID NO: 11 is plant enhancer eZm00001d035657 (eZm11).
SEQ ID NO: 12 is plant enhancer eZm00001d035693 (eZm12).
SEQ ID NO: 13 is plant enhancer eZm00001d039526 (eZm13).
SEQ ID NO: 14 is plant enhancer eZm00001d041480 (eZm14).
SEQ ID NO: 15 is plant enhancer eZm00001d045635 (eZm15).
SEQ ID NO: 16 is plant enhancer eZm00001d046472 (eZm16).
SEQ ID NO: 17 is plant enhancer eZm00001d048707 (eZm17).
SEQ ID NO: 18 is plant enhancer eZm00001d052734 (eZm18).
SEQ ID NO: 19 is plant enhancer eZm00001d053090 (eZm19).
SEQ ID NO: 20 is viral enhancer eMSV8.
SEQ ID NO: 21 is plant enhancer eZm00001d050403 (eZm21).
SEQ ID NO: 22 is plant enhancer eZm00001d015463 (eZm22).
SEQ ID NO: 23 is enhancer eNOS.
SEQ ID NO: 24 is viral enhancer eFMV.
SEQ ID NO: 25 is viral enhancer e35S.
SEQ ID NO: 26 is viral enhancer eCsVMV.
SEQ ID NO: 27 is viral enhancer eCvi.
SEQ ID NO: 28 is viral enhancer eFMV.
SEQ ID NO: 29 is viral enhancer eFMVsg.
SEQ ID NO: 30 is viral enhancer eMMV.
SEQ ID NO: 31 is viral enhancer eCmYLCV.
SEQ ID NO: 32 is viral enhancer ePCISV.
SEQ ID NO: 33 is viral enhancer eRTBV.
SEQ ID NO: 34 is viral enhancer eScBV.
SEQ ID NO: 35 is viral enhancer eScBVIM.
SEQ ID NO: 36 is viral enhancer eBBRV.
SEQ ID NO: 37 is viral enhancer eCERV-s.
SEQ ID NO: 38 is viral enhancer eCERV-L.
SEQ ID NO: 39 is viral enhancer eCoYMV.
SEQ ID NO: 40 is viral enhancer eDCMV_p2.
SEQ ID NO: 41 is viral enhancer eMSV1.
SEQ ID NO: 42 is viral enhancer eMSV2.
SEQ ID NO: 43 is viral enhancer eMSV3.
SEQ ID NO: 44 is viral enhancer eMSV4.
SEQ ID NO: 45 is viral enhancer eMSV5.
SEQ ID NO: 46 is viral enhancer eMSV6.
SEQ ID NO: 47 is viral enhancer eMSV7.
SEQ ID NO: 48 is viral enhancer eMDV.
SEQ ID NO: 49 is viral double enhancer e35s:eFMV.
SEQ ID NO: 50 is plant-viral combination double enhancer eZm21:eScBV
SEQ ID NO: 51 is viral double enhancer eCsVMV:eNOS.
SEQ ID NO: 52 is viral double enhancer eCsVMV:eScBV.
SEQ ID NO: 53 is viral double enhancer eCsVMV:eMMV.
SEQ ID NO: 54 is viral-bacterial combination double enhancer eCvi:eNOS.
SEQ ID NO: 55 is viral double enhancer eCvi:eCsVMV.
SEQ ID NO: 56 is viral-bacterial combination double enhancer eMMV:eNOS.
SEQ ID NO: 57 is viral double enhancer eMMV:eScBV.
SEQ ID NO: 58 is viral double enhancer eMMV:eCsVMV.
SEQ ID NO: 59 is viral double enhancer eCmYLCV:ePCISV.
SEQ ID NO: 60 is viral double enhancer eCmYLCV:eScBV.
SEQ ID NO: 61 is viral double enhancer eCmYLCV:eCvi.
SEQ ID NO: 62 is viral double enhancer ePCISV:eScBV.
SEQ ID NO: 63 is viral double enhancer ePCISV:eCsVMV.
SEQ ID NO: 64 is viral double enhancer ePCISV:eCvi.
SEQ ID NO: 65 is viral double enhancer eFMVsg:ePCISV.
SEQ ID NO: 66 is plant-viral combination double enhancer eZm1:eCsVMV.
SEQ ID NO: 67 is plant-viral combination double enhancer eCsVMV:eZm1.
SEQ ID NO: 68 is plant-viral combination double enhancer eZm1:eMMV.
SEQ ID NO: 69 is plant-viral combination double enhancer eZm3:eMMV.
SEQ ID NO: 70 is plant-viral combination double enhancer eMMV:eZm3.
SEQ ID NO: 71 is plant-viral combination double enhancer eZm8:eScBV.
SEQ ID NO: 72 is plant-viral combination double enhancer eScBV:eZm8.
SEQ ID NO: 73 is plant-viral combination double enhancer eZm5:eScBV.
SEQ ID NO: 74 is plant-bacterial combination double enhancer eZm7:eNOS.
SEQ ID NO: 75 is plant-viral combination double enhancer eZm11:eFMV.
SEQ ID NO: 76 is plant-viral combination double enhancer eZm14:eCsVMV.
SEQ ID NO: 77 is plant-viral combination double enhancer eZm15:eCvi.
SEQ ID NO: 78 is plant-viral combination double enhancer eZm18:eMMV.
SEQ ID NO: 79 is plant-viral combination double enhancer eZm22:eCmYLCV.
SEQ ID NO: 80 is plant-viral combination double enhancer eZm19:ePCISV.
SEQ ID NO: 81 is viral double enhancer eFMV-06:e35s.
SEQ ID NO: 82 is intron iBdUbi10.
SEQ ID NO: 83 is intron iBdEF1a.
SEQ ID NO: 84 is intron iUbi1.
SEQ ID NO: 85 is intron iPvUbi1.
SEQ ID NO: 86 is intron iZmGRMZM2G088088.
SEQ ID NO: 87 is intron iZmABP3.
SEQ ID NO: 88 is intron iZmGRMZM2G149768.
SEQ ID NO: 89 is intron iZm061393.
SEQ ID NO: 90 is intron iZm009722.
SEQ ID NO: 91 is intron iZmHSP70.
SEQ ID NO: 92 is intron iUbi1.
SEQ ID NO: 93 is plant terminator tOsRubiq2.
SEQ ID NO: 94 is plant terminator tZmABP.
SEQ ID NO: 95 is plant terminator tZmUGTBx9.
SEQ ID NO: 96 is plant terminator tPvUbi1.
SEQ ID NO: 97 is plant terminator tOsAct1.
SEQ ID NO: 98 is plant terminator tUbi1.
SEQ ID NO: 99 is plant terminator tSoUbi4.
SEQ ID NO: 100 is plant terminator tBdEF1a.
SEQ ID NO: 101 is terminator tNOS from Agrobacterium Nopaline Synthase gene.
SEQ ID NO: 102 is promoter prZm061393.
SEQ ID NO: 103 is the coding sequence for a truncated, maize optimized Cry1Ab gene.
SEQ ID NO: 104 is the polypeptide sequence for a truncated, maize optimized Cry1Ab protein.
SEQ ID NO: 105 is the DNA sequence for the RCC3 promoter.
SEQ ID NO: 106 is the DNA sequence for the RCC3 terminator.
SEQ ID NO: 107 is the DNA sequence for the FR8A gene.

### Definitions

Although the following terms are believed to be well understood by one of ordinary skill in the art, the following definitions are set forth to facilitate understanding of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

All patents, patent publications, non-patent publications referenced herein are incorporated by reference in their entireties for the teachings relevant to the sentence or paragraph in which the reference is presented. In case of a conflict in terminology, the present specification is controlling.

As used herein, the terms “a” or “an” or “the” may refer to one or more than one, unless the context clearly and unequivocally indicates otherwise. For example, “an” endogenous nucleic acid can mean one endogenous nucleic acid or a plurality of endogenous nucleic acids.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means ±1° C., preferably +0.5° C. Where the term “about” is used in the context of this invention (e.g., in combinations with temperature or molecular weight values) the exact value (i.e., without “about”) is preferred.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above.

As used herein, a “biologically active fragment” refers to a fragment of a reference sequence that has activity that is substantially equivalent to (e.g., at least 90% equivalent to) or greater than the activity of a reference sequence. For example, a biologically active fragment of a reference promoter would be a fragment that is capable of driving expression of a coding sequence at a substantially equivalent or higher level compared to the reference promoter. In some instances, a biologically active fragment may also be referred to as a “functional fragment”.

A “coding sequence” is a nucleic acid sequence that is transcribed into RNA such as mRNA, rRNA, tRNA, snRNA, sense RNA or antisense RNA. In some embodiments, the RNA is then translated in an organism to produce a protein.

As used herein, the terms “elite” and/or “elite line” refer to any line that is substantially homozygous and has resulted from breeding and selection for desirable agronomic performance.

A “transcriptional enhancer” (herein also referred to as “enhancer”, “enhancer sequence” or “enhancer element”) is a nucleotide sequence that can stimulate transcription, such as by stimulating promoter activity. The transcriptional enhancer can be an innate element of the promoter or a heterologous element inserted to enhance the transcriptional level and/or tissue specificity of an associated promoter. The primary sequence of the transcriptional enhancer can be present on either strand of a double-stranded DNA molecule and is capable of functioning even when placed either upstream or downstream from the promoter. Typically, transcriptional enhancers are 50-1000 bps in length and are bound by activator proteins, including transcription factors. However, some enhancers, such as plant enhancers, may be larger than 1 kb in size. When bound, they result in an accessible chromatic structure which promotes gene expression. Enhancer sequences are distinct from sequences corresponding to promoters and transcription start sites. In some examples, enhancers include GC rich motifs.

Enhancers augment transcriptional activity from an operably linked promoter and heterologous polynucleotide sequence by at least 10%, relative to transcriptional values obtained without the enhancer. As non-limiting examples, the inclusion of an enhancer, such as the enhancer elements of the present disclosure, can augment transcription of a gene of interest via an operably linked promoter by at least 10%, at least 20%, at least 50%, at least 100%, at least 150%, at least 200%, at least 300%, or any value there-between. Still higher levels of transcription are also possible.

Enhancers may be identified by applying a computational approach and by using a combination of genomic and epigenomic technologies. For example, sequence conservation of non-coding regions can be used to predict the presence of enhancers. By comparing the sequence data to experimental data, such as one or more of measured DNA methylation levels, DNase sensitivity, histone modification levels, specific chromatin features (such as measured chromatin accessibility) and differential expression levels of nucleotide sequences, enhancer sequences and their putative targets may be predicted with higher accuracy.

Enhancer sequences may be classified based on their source of origin. For example, plant enhancer sequences used herein refer to enhancer sequences retrieved from plant genomic data that, in their natural state, promote transcription of plant genes from plant promoters. As another example, viral enhancer sequences used herein refer to enhancer sequences retrieved from viral genomic data that, in their natural state, promote transcription of viral genes from viral promoters. Enhancer sequences may be heterologous to a promoter they are operably linked to in an expression cassette to induce the expression of a polynucleotide sequence. That is, the enhancer is derived from a different organism than the promoter which it is enhancing, as well as the gene whose expression it is enhancing. Further, in expression cassettes comprising multiple enhancers operably linked to each other and operably linked to a promoter, the enhancer elements may be heterologous to each other. For example, a viral enhancer may be operably linked to a plant enhancer in an expression cassette.

The term “chimeric enhancer” or “chimeric transcriptional enhancer” as used herein refers to the juxtaposition of multiple (e.g., two or more) transcriptional enhancer elements in a construct, wherein each of the multiple enhancer elements are operably coupled to the same promoter. In one example embodiment, the multiple enhancer elements are heterologous to each other; that is, derived from different sources. Heterologous enhancers may include enhancer elements derived from different organisms (e.g., bacterial versus viral versus plant). Alternatively, heterologous enhancers may include enhancer elements derived from different species of the same organism (e.g., multiple plant enhancers from different plant species; or multiple viral enhancers from different virus species). Typically, the multiple transcriptional enhancer elements are positioned abutting each other in the chimeric enhancer with minimal space between them (e.g., no space between them or separated by one or more restriction site sequences). Each of the enhancer elements of the chimeric enhancer are positioned upstream of a common promoter and a coding sequence driven by the promoter in the construct. The enhancer elements included in the chimeric enhancer may include any combination of plant, viral, bacterial, and synthetic enhancers. In some examples, the chimeric enhancer comprises multiple copies (e.g., two, three, or more copies) of the same enhancer element positioned consecutively. In other examples, the chimeric enhancer comprises at least a first enhancer element contiguous to a second, different enhancer element. Additional enhancer elements may also be present. The at least first and at least second enhancer elements may both be plant enhancers or viral enhancers or bacterial enhancers. Alternatively, the chimeric enhancer may comprise at least a plant enhancer juxtaposed next to a viral enhancer, such as a plant enhancer upstream of the viral enhancer, or a viral enhancer upstream of a plant enhancer. As another example, the chimeric enhancer may comprise at least a plant enhancer juxtaposed next to a bacterial enhancer, such as a plant enhancer upstream of the bacterial enhancer, or a bacterial enhancer upstream of a plant enhancer. As yet another example, the chimeric enhancer may comprise at least a bacterial enhancer, a viral enhancer, a plant enhancer, or fragments thereof, in any combination.

The term “double enhancer” as used herein refers to the juxtaposition of two transcriptional enhancer elements in a construct, wherein each of the two enhancer elements are operably coupled to the same promoter. Typically, the enhancer elements are positioned abutting each other with a minimal space between them (e.g., no space between them or separated by one or more restriction site sequences). Both enhancer elements of the double enhancer are positioned upstream of a promoter and a coding sequence driven by the promoter in the construct. The enhancers included in the double enhancer may include any combination of plant, viral, bacterial, and synthetic enhancers. In some examples, the double enhancer comprises two copies of the same enhancer element positioned consecutively. In other examples, the double enhancer comprises a first enhancer element contiguous to a second, different enhancer element. The first and second enhancer elements may both be plant enhancers or viral enhancers or bacterial enhancers. Alternatively, the double enhancer may comprise a plant enhancer juxtaposed next to a viral enhancer, such as a plant enhancer upstream of the viral enhancer, or a viral enhancer upstream of a plant enhancer. As another example, the double enhancer may comprise a plant enhancer juxtaposed next to a bacterial enhancer, such as a plant enhancer upstream of the bacterial enhancer, or a bacterial enhancer upstream of a plant enhancer. While the double enhancers of the present disclosure are illustrated herein via combinations of viral and plant enhancers, this is not meant to be limiting. In further examples, any combination of viral, bacterial or plant transcriptional enhancer elements (including identified or putative enhancer elements) may be used without departing from the scope of the invention.

The term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g., if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In embodiments, “expression” refers to the transcription and stable accumulation of sense (mRNA) or functional RNA. “Expression” may also refer to the production of protein.

“Expression cassette” as used herein means a nucleic acid molecule capable of directing expression of a particular polynucleotide or polynucleotides in an appropriate host cell, comprising a promoter operably linked to the polynucleotide or polynucleotides of interest which is/are operably linked to termination signals. It also typically comprises polynucleotides required for proper translation of the polynucleotide or polynucleotides of interest. Further, the expression cassette can include one or more enhancers operably linked to the promoter to augment activity (general or tissue-specific activity) of the promoter, and thereby transcription of the polynucleotide sequence driven by the promoter. The expression cassette may also comprise polynucleotides not necessary in the direct expression of a polynucleotide of interest, but which are present due to convenient restriction sites for removal of the cassette from an expression vector. The expression cassette comprising the polynucleotide(s) of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e. the particular polynucleotide of the expression cassette does not occur naturally in the host cell and must have been introduced into the host cell or an ancestor of the host cell by a transformation process known in the art. The expression of the polynucleotide(s) in the expression cassette is generally under the control of a promoter. In the case of a multicellular organism, such as a plant, the promoter can also be specific or preferential to a particular tissue, or organ, or stage of development. An expression cassette, or fragment thereof, can also be referred to as “inserted polynucleotide” or “insertion polynucleotide” when transformed into a plant.

As used herein, the term “genome editing agent” refers to an agent that is capable of inducing a deletion, insertion, indel, or other modification in the genome of a cell, e.g., by creating a single or double-stranded break in the genome. Examples of genome editing agents include CRISPR/Cas agents (e.g., Cas proteins and guide RNAs), transcription activator-like effector nucleases (TALENs), DNA-guided nucleases, meganucleases, recombinases, and zinc finger nucleases. Cas proteins include Cas9, Cpf1 (also known as Cas12a), C2c1, C2c2, and C2c3, and functional variants thereof. Example Cas9 and Cpf1 proteins include Streptococcus pyogenes Cas9 (SpCas9), Streptococcus thermophilus Cas9 (StCas9), Streptococcus pasteurianus (SpaCas9), Campylobacter jejuni Cas9 (CjCas9), Staphylococcus aureus (SaCas9), Francisella novicida Cas9 (FnCas9), Neisseria cinerea Cas9 (NcCas9), Neisseria meningitis Cas9 (NmCas9), Francisella novicida Cpf1 (FnCpf1), Acidaminococcus sp. Cpf1 (AsCpf1), or Lachnospiraceae bacterium ND2006 Cpf1 (LbCpf1). A “variant” of a Cas protein refers to a protein or polypeptide derivative of a wild type Cas protein, e.g., a protein having one or more point mutations, insertions, deletions, truncations, a fusion protein, or a combination thereof. In certain embodiments, the Cas variant is a functional variant which substantially retains the nuclease activity of or has better nuclease activity than the wild type Cas protein. Example guide RNAs include single guide RNAs and dual guide RNAs.

A “heterologous” nucleic acid sequence is a nucleic acid sequence not naturally associated with a host cell into which it is introduced, including non-naturally occurring multiple copies of a naturally occurring nucleic acid sequence. A nucleic acid sequence can also be heterologous to other nucleic acid sequences with which it may be associated, for example in a nucleic acid construct, such as e.g., an expression vector. As one nonlimiting example, a promoter may be present in a nucleic acid construct in combination with one or more regulatory element (e.g., enhancer element) and/or coding sequences that do not naturally occur in association with that particular promoter, i.e., they are heterologous to the promoter.

In some embodiments, the heterologous sequence is a nucleic acid or gene of interest that encodes an RNA or protein of interest. In some embodiments, the RNA or protein of interest is capable of conferring upon a plant a desired characteristic such as antibiotic resistance, virus resistance, insect resistance, disease resistance, resistance to other pests, herbicide tolerance, improved nutritional value, improved performance in an industrial process, and altered reproductive capability. As one example, such as in the constructs described herein, the gene of interest that is operably coupled to a promoter and an enhancer sequence is truncated, maize optimized Cry1Ab gene that codes for a Bt toxin protein that confers resistance to insects (e.g., one or more insects of order Lepidoptera).

As used herein, the term “intron” refers to a nucleotide sequence provided within a gene (that is, in an intragenic region) and that is removed by splicing during maturation of a final RNA product. Thus, introns are non-coding regions of an RNA transcript, or the DNA encoding it. Introns separate exons such that splicing results in removal of introns and joining of exons. Introns are marked by the presence of conserved sequences known as splice sites at 5′ and 3′ ends. Typically, the splice site at the 5′ end includes an AG sequence and the splice site at the 3′ end includes a GU sequence. Splicing of the introns is catalyzed by a spliceosome comprising RNA and proteins. Promoter sequences can, in some embodiments (e.g., for larger promoter sequences such as those for proximal or distal promoters) include an intron. In other embodiments, as described with reference to the constructs disclosed herein, introns may be optionally coupled to a minimal or core promoter sequence.

An “isolated” nucleic acid molecule or nucleotide sequence or an “isolated” polypeptide is a nucleic acid molecule, nucleotide sequence or polypeptide that, by the hand of man, exists apart from its native environment and/or has a function that is different, modified, modulated and/or altered as compared to its function in its native environment and is therefore not a product of nature. An isolated nucleic acid molecule or isolated polypeptide may exist in a purified form or may exist in a non-native environment such as, for example, a recombinant host cell. Thus, for example, with respect to polynucleotides, the term isolated means that it is separated from the chromosome and/or cell in which it naturally occurs. A polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and nucleotide sequences of the invention can be considered to be “isolated” as defined above.

Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a translation start site or transcription start site of a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid molecule encoding an additional polypeptide or peptide sequence. An “isolated nucleic acid molecule” or “isolated nucleotide sequence” can also include a nucleotide sequence derived from and inserted into the same natural, original cell type, but which is present in a non-natural state, e.g., present in a different copy number, and/or under the control of different regulatory sequences than that found in the native state of the nucleic acid molecule.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not necessarily mean that the preparation is technically pure (homogeneous), but it is sufficiently pure to provide the polypeptide or nucleic acid in a form in which it can be used for the intended purpose.

In representative embodiments of the invention, an “isolated” nucleic acid molecule, nucleotide sequence, and/or polypeptide is at least about 5%, 10%, 15%, 20%, 25%, 30%, 40%, 50%, 60%, 70%, 75%, 80%, 85%, 90%, 95%, 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10,000-fold, 100,000-fold or more enrichment of the nucleic acid (w/w) is achieved as compared with the starting material.

The term “introducing” or “introduce” in the context of a plant cell, plant and/or plant part means contacting a nucleic acid molecule with the plant, plant part, and/or plant cell in such a manner that the nucleic acid molecule gains access to the interior of the plant cell and/or a cell of the plant and/or plant part. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol. Thus, the term “transformation” as used herein refers to the introduction of a heterologous nucleic acid into a cell. Transformation of a cell may be stable or transient. Thus, a transgenic plant cell, plant and/or plant part of the invention can be stably transformed or transiently transformed. In a transient transformation, the introduced heterologous nucleic acid does not get integrated into the genome of the transgenic plant cell, plant and/or plant part. In a stable transformation, the introduced heterologous nucleic acid does get integrated into the genome of the transgenic plant cell, plant and/or plant part and can be passed on to future generations of the cell.

The terms “percent sequence identity” or “percent identity” are used interchangeably herein and is used herein refers to the percentage of identical nucleotides or amino acids in a linear polynucleotide or amino acid sequence of a reference (“query”) sequence (or its complementary strand) as compared to a test (“subject”) sequence (or its complementary strand) when the two sequences are optimally aligned. Optimal alignment of sequences for aligning a comparison window are known to those skilled in the art and may be conducted using known methods, e.g., using known software or computer programs such as the Smith and Waterman algorithm implemented in the EMBOSS-6.6.0 water tool using default matrix files EBLOSUM62 for protein, EDNAFULL for DNA with default gap penalties. EMBOSS-6.6.0 is available, e.g., from the following Bio-soft and Open-Bio such as at the following websites: en.bio-soft.net/format/emboss.html or emboss.open-bio.org/html/adm/ch01s01.html.

The terms “nucleic acid” or “polynucleotide” are used interchangeably herein and refer to any physical string of monomer units that can be corresponded to a string of nucleotides, including a polymer of nucleotides (e.g., a typical DNA polymer or polydeoxyribonucleotide or RNA polymer or polyribonucleotide), modified oligonucleotides (e.g., oligonucleotides comprising bases that are not typical to biological RNA or DNA, such as 2′-O-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated. The nucleic acid can be present in a vector, such as in a cell, virus or plasmid.

“Operably linked” refers to the association of polynucleotides on a single nucleic acid fragment so that the function of one affects the function of the other. For example, a promoter is operably linked with a coding polynucleotide when it is capable of affecting the expression of that coding polynucleotide (i.e., that the coding polynucleotide is under the transcriptional control of the promoter). As another example, one or more enhancers are operably linked with a promoter when the enhancer(s) are capable of affecting the binding of transcription factors to the promoter and thereby augmenting the expression of a coding polynucleotide under the transcriptional control of the promoter. Coding polynucleotide in sense or antisense orientation can be operably linked to regulatory polynucleotides. Enhancers can be operably linked to the promoter in a sense or antisense orientation.

The term “plant” refers to any plant, particularly to agronomically useful plants (e.g. seed plants), and “plant cell” is a structural and physiological unit of the plant, which comprises a cell wall but may also refer to a protoplast. The plant cell may be in form of an isolated single cell or a cultured cell, or as a part of higher organized units such as for example, a plant tissue, or a plant organ differentiated into a structure that is present at any stage of a plant's development. A plant may be a monocotyledonous or dicotyledonous plant species.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

The term “plant part,” as used herein, includes but is not limited to embryos, pollen, ovules, seeds, leaves, stems, shoots, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell of the present invention can be in the form of an isolated single cell or can be a cultured cell or can be a part of a higher-organized unit such as, for example, a plant tissue or a plant organ.

“Plant tissue” as used herein means a group of plant cells organized into a structural and functional unit. Any tissue of a plant in planta or in culture is included. This term includes, but is not limited to, whole plants, plant organs, plant seeds, tissue culture and any group of plant cells organized into structural and/or functional units. The use of this term in conjunction with, or in the absence of, any specific type of plant tissue as listed above or otherwise embraced by this definition is not intended to be exclusive of any other type of plant tissue.

The term “promoter,” as used herein, refers to a polynucleotide, usually upstream (5′) of the translation start site of a coding sequence, which controls the expression of the coding sequence by providing the recognition for RNA polymerase and other factors required for proper transcription. For example, a promoter may contain a region containing basal promoter elements recognized by RNA polymerase, a region containing the 5′ untranslated region (UTR) of a coding sequence, and optionally an intron. In some embodiments, a promoter comprises or consists of the about 2 kb region upstream (5′) of the translation start site of a known or predicted coding sequence. In some embodiments, such as in the constructs described herein, the promoter is a minimal or core promoter comprising only those elements that are required to initiate transcription. For example, a minimal promoter may consist of a transcription start site (TSS), a binding site for RNA polymerase, and a transcription factor binding site (such as a TATA box or B recognition element). Such minimal promoter may not comprise any introns or splice sites.

“Regulatory elements” and “regulatory sequences” are used interchangeably herein and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include transcriptional enhancers, promoters, translational enhancer sequences, introns, terminators, and polyadenylation signal sequences. They include natural and synthetic sequences as well as sequences which may be a combination of synthetic and natural sequences. Regulatory sequences may determine expression level, the spatial and temporal pattern of expression and, for a subset of promoters, expression under inductive conditions (regulation by external factors such as light, temperature, chemicals and hormones). Regulatory sequences may be short regions of DNA sequence 6-100 base pairs that define the binding sites for trans-acting factors, such as transcription factors. Regulatory sequences may also be enhancers, longer regions of DNA sequence that can act from a distance from the core promoter region, sometimes over several kilobases from the core region. Regulatory sequence activity may be influenced by trans-acting factors including general transcription machinery, transcription factors and chromatin assembly factors.

A “terminator,” as used herein, is responsible for the termination of transcription beyond the translation stop site of a coding sequence and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV 35S terminator, the tml terminator, the nopaline synthase terminator and the pea rbcs E9 terminator. These can be used in both monocotyledons and dicotyledons. In addition, a gene's native transcription terminator may be used. For example, a terminator may contain a region containing the 3′ untranslated region (UTR) of a coding sequence, and optionally additional 3′ non-transcribed sequence. In some embodiments, a terminator comprises or consists of the about 1 kb region downstream (3′) of the translation stop site of a known or predicted coding sequence.

A “selectable marker” or “selectable marker gene” refers to a gene whose expression in a plant cell gives the cell a selective advantage. “Positive selection” refers to a transformed cell acquiring the ability to metabolize a substrate that it previously could not use or could not use efficiently, typically by being transformed with and expressing a positive selectable marker gene. This transformed cell thereby grows out of the mass of non-transformed tissue. Positive selection can be of many types from inactive forms of plant growth regulators that are then converted to active forms by the transferred enzyme to alternative carbohydrate sources that are not utilized efficiently by the non-transformed cells, for example mannose, which then become available upon transformation with an enzyme, for example phosphomannose isomerase, that allows them to be metabolized. Non-transformed cells either grow slowly in comparison to transformed cells or not at all. Other types of selection may be due to the cells transformed with the selectable marker gene gaining the ability to grow in presence of a negative selection agent, such as an antibiotic or an herbicide, compared to the ability to grow of non-transformed cells. A selective advantage possessed by a transformed cell may also be due to the loss of a previously possessed gene in what is called “negative selection”. In this, a compound is added that is toxic only to cells that did not lose a specific gene (a negative selectable marker gene) present in the parent cell (typically a transgene).

Examples of selectable markers include, but are not limited to, genes that provide resistance or tolerance to antibiotics such as kanamycin (Dekeyser et al. 1989, Plant Phys 90: 217-23), spectinomycin (Svab and Maliga 1993, Plant Mol Biol 14: 197-205), streptomycin (Maliga et al. 1988, Mol Gen Genet 214: 456-459), hygromycin B (Waldron et al. 1985, Plant Mol Biol 5: 103-108), bleomycin (Hille et al. 1986, Plant Mol Biol 7: 171-176), sulphonamides (Guerineau et al. 1990, Plant Mol Biol 15: 127-136), streptothricin (Jelenska et al. 2000, Plant Cell Rep 19: 298-303), or chloramphenicol (De Block et al. 1984, EMBO J 3: 1681-1689). Other selectable markers include genes that provide resistance or tolerance to herbicides, such as the S4 and/or Hra mutations of acetolactate synthase (ALS) that confer resistance to herbicides including sulfonylureas, imidazolinones, triazolopyrimidines, and pyrimidinyl thiobenzoates; 5-enol-pyrovyl-shikimate-3-phosphate-synthase (EPSPS) genes, including but not limited to those described in U.S. Pat. Nos. 4,940,935, 5,188,642, 5,633,435, 6,566,587, 7,674,598 (as well as all related applications) and the glyphosate N-acetyltransferase (GAT) which confers resistance to glyphosate (Castle et al. 2004, Science 304:1151-1154, and U.S. Patent Application Publication Nos. 20070004912, 20050246798, and 20050060767); BAR which confers resistance to glufosinate (see e.g., U.S. Pat. Nos. 5,561,236); aryloxy alkanoate dioxygenase or AAD-1, AAD-12, or AAD-13 which confer resistance to 2,4-D; genes such as Pseudomonas HPPD which confer HPPD resistance; Sprotophorphyrinogen oxidase (PPO) mutants and variants, which confer resistance to peroxidizing herbicides including fomesafen, acifluorfen-sodium, oxyfluorfen, lactofen, fluthiacet-methyl, saflufenacil, flumioxazin, flumiclorac-pentyl, carfentrazone-ethyl, sulfentrazone); and genes conferring resistance to dicamba, such as dicamba monoxygenase (Herman et al. 2005, J Biol Chem 280: 24759-24767 and U.S. Pat. No. 7,812,224 and related applications and patents). Other examples of selectable markers can be found in Sundar and Sakthivel (2008, J Plant Physiology 165: 1698-1716), herein incorporated by reference.

Other selection systems include using drugs, metabolite analogs, metabolic intermediates, and enzymes for positive selection or conditional positive selection of transgenic plants. Examples include, but are not limited to, a gene encoding phosphomannose isomerase (PMI) where mannose is the selection agent, or a gene encoding xylose isomerase where D-xylose is the selection agent (Haldrup et al. 1998, Plant Mol Biol 37: 287-96). Finally, other selection systems may use hormone-free medium as the selection agent. One non-limiting example the maize homeobox gene kn1, whose ectopic expression results in a 3-fold increase in transformation efficiency (Luo et al. 2006, Plant Cell Rep 25: 403-409). Examples of various selectable markers and genes encoding them are disclosed in Miki and McHugh (J Biotechnol, 2004, 107: 193-232; incorporated by reference).

In some embodiments of the disclosure, the selectable marker may be plant derived. An example of a selectable marker which can be plant derived includes, but is not limited to, 5-enolpyruvylshikimate-3-phosphate synthase (EPSPS). The enzyme 5-enolpyruvylshikimate-3-phosphate synthase (EPSPS) catalyzes an essential step in the shikimate pathway common to aromatic amino acid biosynthesis in plants. The herbicide glyphosate inhibits EPSPS, thereby killing the plant. Transgenic glyphosate-tolerant plants can be created by the introduction of a modified EPSPS transgene which is not affected by glyphosate (for example, U.S. Pat. No. 6,040,497; incorporated by reference). Other examples of a modified plant EPSPS which can be used as a selectable marker in the presence of glyphosate includes a P106L mutant of rice EPSPS (Zhou et al 2006, Plant Physiol 140: 184-195) and a P106S mutation in goosegrass EPSPS (Baerson et al 2002, Plant Physiol 129: 1265-1275). Other sources of EPSPS which are not plant derived and can be used to confer glyphosate tolerance include but are not limited to an EPSPS P101S mutant from Salmonella typhimurium (Comai et al 1985, Nature 317: 741-744) and a mutated version of CP4 EPSPS from Agrobacterium sp. Strain CP4 (Funke et al 2006, PNAS 103: 13010-13015). Although the plant EPSPS gene is nuclear, the mature enzyme is localized in the chloroplast (Mousdale and Coggins 1985, Planta 163:241-249). EPSPS is synthesized as a preprotein containing a transit peptide, and the precursor is then transported into the chloroplast stroma and proteolytically processed to yield the mature enzyme (della-Cioppa et al. 1986, PNAS 83: 6873-6877). Therefore, to create a transgenic plant which has tolerance to glyphosate, a suitably mutated version of EPSPS which correctly translocates to the chloroplast could be introduced. Such a transgenic plant then has a native, genomic EPSPS gene as well as the mutated EPSPS transgene. Glyphosate could then be used as a selection agent during the transformation and regeneration process, whereby only those plants or plant tissue that are successfully transformed with the mutated EPSPS transgene survive.

The term “transformation” as used herein refers to the transfer of a nucleic acid into a host cell, preferably resulting in genetically stable integration, which includes integration into a chromosome and heritable extrachromosomal events. In some particular embodiments, the introduction into a plant, plant part and/or plant cell is via bacterial-mediated transformation, particle bombardment transformation (also called biolistic particle transformation), calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of a nucleic acid into the plant, plant part and/or cell thereof, or a combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (2002, Cell Mol Biol Lett 7:849-858 (2002)).

Procedures for transforming plants are well known and routine in the art and are described throughout the literature. Non-limiting examples of methods for transformation of plants include transformation via bacterial-mediated nucleic acid delivery (e.g., via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell Mol Biol Lett 7:849-858 (2002)).

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. 1993, Plant Cell 5:159-169). The transfer of the recombinant binary vector to Agrobacterium can be accomplished by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen and Willmitzer 1988, Nucleic Acids Res 16:9877).

Transformation of a plant by recombinant Agrobacterium usually involves co-cultivation of the Agrobacterium with explants from the plant and follows methods well known in the art. Transformed tissue is typically regenerated on selection medium carrying an antibiotic or herbicide resistance marker between the binary plasmid T-DNA borders.

Another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacteria or a bacteriophage, each containing one or more nucleic acids sought to be introduced) also can be propelled into plant tissue.

Thus, in particular embodiments of the present invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

A “transgenic plant” is a plant having one or more plant cells that contain a heterologous DNA sequence.

As used herein, “vector” includes reference to a nucleic acid used in transfection of a host cell and into which can be inserted a polynucleotide. Vectors are often replicons. Expression vectors permit transcription of a nucleic acid inserted therein. “Vector” is defined to include, inter alia, any plasmid, cosmid, phage or Agrobacterium binary vector in double or single stranded linear or circular form which may or may not be self-transmissible or mobilizable, and which can transform prokaryotic or eukaryotic host either by integration into the cellular genome or exist extrachromosomally (e.g. autonomous replicating plasmid with an origin of replication). Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

### DETAILED DESCRIPTION

Aspects of the disclosure relate to regulatory elements, such as transcriptional enhancers, introns, and terminators, useful for expression of heterologous sequences in plants, such as maize.

In one embodiment, the disclosure provides an expression cassette comprising two operably linked nucleotide sequences. In one aspect of the embodiment, the first nucleotide sequence is a plant transcriptional enhancer. In another aspect of this embodiment, the second nucleotide sequence is a viral transcriptional enhancer. In further aspects, the operably linked transcriptional enhancers are adjacent or contiguous to each other and the expression cassette comprises a third nucleotide sequence having at least 90% identity (e.g., at least 91%, at least 92%, at least 93%, at least 94%, at least 95%, at least 96%, at least 97%, at least 98%, at least 99%, or 100% identity) with one or more of SEQ ID NOs: 49-80. In another aspect of this embodiment, the third nucleotide sequence comprises one or more of SEQ ID NOs: 49-80. In yet another aspect, the operably linked transcriptional enhancers are separated only by one or more restriction enzyme digestion sites.

In another embodiment, the disclosure provides a nucleotide sequence that is a plant transcriptional enhancer having at least 90% identity (e.g., at least 91%, at least 92%, at least 93%, at least 94%, at least 95%, at least 96%, at least 97%, at least 98%, at least 99%, or 100% identity) with one or more of SEQ ID NOs: 1-21. In one aspect of this embodiment, the plant transcriptional enhancer comprises one or more of SEQ ID NOs: 1-21.

In another embodiment, the disclosure provides a nucleotide sequence that is a viral transcriptional enhancer having at least 90% identity (e.g., at least 91%, at least 92%, at least 93%, at least 94%, at least 95%, at least 96%, at least 97%, at least 98%, at least 99%, or 100% identity) with one or more of SEQ ID NOs: 22-48. In one aspect of this embodiment, the viral transcriptional enhancer comprises one or more of SEQ ID NOs: 22-48.

In another embodiment, the disclosure provides a plant transcriptional enhancer that is selected from the group consisting of SEQ ID NO: 18, SEQ ID NO: 19, SEQ ID NO: 8, SEQ ID NO: 11, and SEQ ID NO: 12.

In yet another embodiment, the disclosure provides a viral transcriptional enhancer that is selected from the group consisting of SEQ ID NO: 30, SEQ ID NO: 32, SEQ ID NO: 26, and SEQ ID NO: 34.

In further embodiments, the disclosure provides an expression cassette comprising two heterologous transcriptional enhancers (herein also referred to as dual transcriptional enhancer sequences) positioned contiguous to each other, wherein the dual transcriptional enhancer sequences are operably linked to an additional heterologous sequence. In one aspect of the embodiment, the additional heterologous sequence comprises a nucleotide sequence having at least 90% identity (e.g., at least 91%, at least 92%, at least 93%, at least 94%, at least 95%, at least 96%, at least 97%, at least 98%, at least 99%, or 100% identity) with one or more of SEQ ID NOs: 82-91. In another aspect of this embodiment, the heterologous sequence comprises one or more of SEQ ID NOs: 82-91.

In some other embodiments, the additional heterologous sequence comprises a nucleotide sequence having at least 90% identity (e.g., at least 91%, at least 92%, at least 93%, at least 94%, at least 95%, at least 96%, at least 97%, at least 98%, at least 99%, or 100% identity) with one or more of SEQ ID NOs: 93-100. In another aspect of this embodiment, the heterologous sequence comprises one or more of SEQ ID NOS: 93-100.

In some embodiments, the heterologous sequence is a nucleic acid of interest that encodes an RNA or protein of interest. In some embodiments, the RNA or protein of interest is capable of conferring upon a plant a desired characteristic such as antibiotic resistance, virus resistance, insect resistance, disease resistance, resistance to other pests, herbicide tolerance, improved nutritional value, improved performance in an industrial process, or altered reproductive capability.

In some embodiments, the expression cassette further comprises a selectable marker.

In some embodiments, the expression cassette is comprised within a vector, such as a plasmid, virus, or Agrobacterium. In other embodiments, the expression cassette is comprised within a plant cell. In some embodiments, the plant cell is a monocot cell. In some embodiments, the plant cell is a dicot cell. In some embodiments, the plant cell is a Zea mays cell. In some embodiments, the Zea mays cell is an elite Zea mays cell.

In some embodiments, the expression cassette is comprised within a transgenic plant. In some embodiments, the plant is a monocot. In some embodiments, the plant is a Zea mays plant. In some embodiments, the Zea mays plant is an elite Zea mays plant. In some embodiments, the plant is a dicot. In some embodiments, the dicot plant is a soy plant cell or a tobacco plant.

In some embodiments, the disclosure provides a seed from a transgenic plant, e.g., a seed comprising the expression cassette.

In still further embodiments, the disclosure provides for a polynucleotide comprising a chimeric enhancer sequence, wherein the chimeric enhancer sequence comprises a first plant enhancer sequence operably linked to a second viral enhancer sequence. In some aspects of this embodiment, the chimeric enhancer sequence has a higher transcriptional enhancing activity on a promoter operably linked to the chimeric enhancer sequence than either enhancer sequence alone. In some embodiments, the polynucleotide further comprises a promoter operably linked to the chimeric enhancer and a heterologous polynucleotide transcribable by the promoter, the heterologous polynucleotide encoding a gene of interest that imparts a desirable trait upon transformation. In still further embodiments, the polynucleotide further comprises a terminator operably linked to the heterologous polynucleotide.

In another embodiment, the disclosure provides for an expression cassette comprising, as operably linked components, a chimeric enhancer comprising a first plant enhancer contiguous to a second viral enhancer; a promoter; a heterologous polynucleotide transcribable by the promoter, wherein transcription of the polynucleotide by the promoter is increased by the chimeric enhancer; and optionally, a terminator. In further representations of this embodiment, the first plant enhancer sequence is a polynucleotide comprising one of SEQ ID NO: 18, SEQ ID NO: 19, SEQ ID NO: 8, SEQ ID NO: 11, and SEQ ID NO: 12; or a polynucleotide having at least 90% sequence identity to one of SEQ ID NO: 18, SEQ ID NO: 19, SEQ ID NO: 8, SEQ ID NO: 11, and SEQ ID NO: 12. In further representations of this embodiment, the second viral enhancer sequence is a polynucleotide comprising one of SEQ ID NO: 30, SEQ ID NO: 32, SEQ ID NO: 26, and SEQ ID NO: 34 or a polynucleotide having at least 90% sequence identity to or comprises one of SEQ ID NO: 30, SEQ ID NO: 32, SEQ ID NO: 26, and SEQ ID NO: 34. In further representations of this embodiment, the promoter of the expression cassette is a plant promoter that comprises SEQ ID NO: 102; or a polynucleotide having at least 90% sequence identity to SEQ ID NO: 102 or a functional fragment of SEQ ID NO: 102. In further representations of this embodiment, the optional terminator included in the expression cassette comprises SEQ ID NO: 101; or a polynucleotide having at least 90% sequence identity to SEQ ID NO: 102.

In some embodiments of the expression cassette, the heterologous polynucleotide comprises SEQ ID no: 103; or a polynucleotide encoding for a polypeptide comprising SEQ ID NO: 104; or a polynucleotide encoding for a polypeptide having at least 90% sequence identity to SEQ ID NO: 104. In further embodiments, the heterologous polynucleotide encodes a selectable marker protein such as a protein providing a visible marker (e.g., GFP, GUS etc.), a protein providing antibiotic resistance, etc.

In further embodiments, the disclosure provides for polynucleotide sequences comprising novel introns. In one example representation of such an embodiment, a recombinant DNA expression cassette comprises a promoter functioning in plants or plant cells; and an intron comprising (a) the sequence of any one of SEQ ID NOS: 82-91; (b) a polynucleotide sequence having at least 95% sequence identity to any one of SEQ ID NOS: 82-91; or (c) a functional equivalent thereof, wherein the functional equivalent confers intron mediated enhancement of transcription and/or translation of a heterologous nucleic acid driven by the promoter.

In other embodiments, the disclosure provides for polynucleotide sequences comprising novel terminators. In one example representation of such an embodiment, a recombinant DNA expression cassette comprises a promoter functioning in plants or plant cells; and a terminator comprising (a) the sequence of any one of SEQ ID NOS: 93-100; (b) a polynucleotide sequence having at least 95% sequence identity to any one of SEQ ID NOS: 93-100; or (c) a functional equivalent thereof, wherein the functional equivalent confers terminator mediated enhancement of transcription and/or translation of a heterologous nucleic acid driven by the promoter.

Other aspects of the disclosure relate to a method, e.g., a transformation method, comprising introducing an expression cassette or vector as described herein into a plant or plant cell. In some embodiments, the introducing comprises Agrobacterium-mediated transformation. In some embodiments, the introducing comprises particle bombardment. In some embodiments of the method, the method further comprises placing the plant or plant cell under conditions whereby an RNA or protein of interest and/or a selectable marker is expressed from the expression cassette or vector. In some embodiments, the conditions are appropriate growth or maintenance conditions for the plant or plant cell. In some embodiments of the method, the method further comprises crossing the plant to a second plant to produce a progeny plant. In some embodiments, the plant or plant cell is a monocot plant or plant cell. In some embodiments, the plant or plant cell is a Zea mays plant or plant cell. In some embodiments, the plant or plant cell is an elite Zea mays plant or plant cell. In some embodiments, the second plant in an elite Zea mays plant.

In some embodiments, a method of evaluating gene expression in a nascent leaf comprising the steps of transforming the expression construct into Agrobacterium, cutting a nine day old Maize plant above the second node and removing leaves, wounding the stem with a needle matrix, wherein a nascent leaf inside the stem is also wounded, infiltrating the transformed agrobacterium into the wounded stem, harvesting the nascent leaf to evaluate gene expression is provided.

Some embodiments provide an expression cassette comprising a transcriptional enhancer operably linked to a promoter sequence, wherein the promoter sequence provides tissue preferred expression, and wherein the transcriptional enhancer increases the tissue preferred expression.

Some embodiments provide an expression cassette comprising a transcriptional enhancer operably linked to a promoter sequence, wherein the promoter sequence provides tissue preferred expression, and wherein the transcriptional enhancer increases the tissue preferred expression, wherein the tissue preferred expression is root preferred.

Some embodiments provide an expression cassette comprising a transcriptional enhancer operably linked to a promoter sequence, wherein the promoter sequence provides tissue preferred expression, and wherein the transcriptional enhancer increases the tissue preferred expression, wherein the transcriptional enhancer is SEQ ID NO. 30.

Some embodiments provide an expression cassette comprising a transcriptional enhancer operably linked to a promoter sequence, wherein the promoter sequence provides tissue preferred expression, and wherein the transcriptional enhancer increases the tissue preferred expression, wherein the promoter sequence is SEQ ID NO. 102.

Some embodiments provide an expression cassette comprising a transcriptional enhancer operably linked to a promoter sequence, wherein the promoter sequence provides tissue preferred expression, and wherein the transcriptional enhancer increases the tissue preferred expression, wherein the protein encoding polynucleotide is transcribed in leaf tissue and not pollen.

Some embodiments provide an expression cassette comprising a transcriptional enhancer operably linked to a promoter sequence, wherein the promoter sequence provides tissue preferred expression, and wherein the transcriptional enhancer increases the tissue preferred expression, wherein the transcriptional enhancer is selected from the group consisting of SEQ ID NOs. 30, 78, 80, 71, 81, 32, 34, and 8. Some embodiments provide an expression cassette comprising a transcriptional enhancer operably linked to a promoter sequence, wherein the promoter sequence provides tissue preferred expression, and wherein the transcriptional enhancer increases the tissue preferred expression, wherein the promoter sequence is SEQ ID NO. 103.

Other embodiments provide, a method for increasing the expression of a native gene, comprising the following steps: (a) introducing into at least one cell of a plant the nucleic acid molecule of an enhancer element; (b) introducing of a site-directed nuclease and a repair matrix into at least one cell of a plant, wherein the site-directed nuclease is able to generate at least one double strand break of the DNA in the genome in the promoter, the 5′ UTR, the first intron, the 3′ UTR, or the terminator, or any other part of the gene or surrounding gene regulatory sequence of the native gene of the at least one cell and the repair matrix comprises the nucleic acid molecule of an enhancer element or a fragment thereof; (c) cultivation of the at least one cell of (b) under conditions that allow a homology-directed repair or a homologous recombination, wherein the nucleic acid molecule is integrated from the repair matrix into the genome of the plant in the promoter, the 5′ UTR, the first intron, the 3′ UTR, or the terminator, or any other part of the gene or surrounding gene regulatory sequence of the native gene; and (d) obtaining the plant having increased expression of the native gene from the at least one cell. Other embodiments provide, a method for increasing the expression of a native gene, comprising the following steps: (a) introducing into at least one cell of a plant the nucleic acid molecule of an enhancer element; (b) introducing of a site-directed nuclease and a repair matrix into at least one cell of a plant, wherein the site-directed nuclease is able to generate at least one double strand break of the DNA in the genome in the promoter, the 5′ UTR, the first intron, the 3′ UTR, or the terminator, or any other part of the gene or surrounding gene regulatory sequence of the native gene of the at least one cell and the repair matrix comprises the nucleic acid molecule of an enhancer element or a fragment thereof; (c) cultivation of the at least one cell of (b) under conditions that allow a homology-directed repair or a homologous recombination, wherein the nucleic acid molecule is integrated from the repair matrix into the genome of the plant in the promoter, the 5′ UTR, the first intron, the 3′ UTR, or the terminator, or any other part of the gene or surrounding gene regulatory sequence of the native gene; and (d) obtaining the plant having increased expression of the native gene from the at least one cell, wherein the enhancer element is selected from the group of SEQ ID NO. 1-81. Other embodiments provide, a method for increasing the expression of a native gene, comprising the following steps: (a) introducing into at least one cell of a plant the nucleic acid molecule of an enhancer element; (b) introducing of a site-directed nuclease and a repair matrix into at least one cell of a plant, wherein the site-directed nuclease is able to generate at least one double strand break of the DNA in the genome in the promoter, the 5′ UTR, the first intron, the 3′ UTR, or the terminator, or any other part of the gene or surrounding gene regulatory sequence of the native gene of the at least one cell and the repair matrix comprises the nucleic acid molecule of an enhancer element or a fragment thereof; (c) cultivation of the at least one cell of (b) under conditions that allow a homology-directed repair or a homologous recombination, wherein the nucleic acid molecule is integrated from the repair matrix into the genome of the plant in the promoter, the 5′ UTR, the first intron, the 3′ UTR, or the terminator, or any other part of the gene or surrounding gene regulatory sequence of the native gene; and (d) obtaining the plant having increased expression of the native gene from the at least one cell, wherein the native gene is selected from the group of the maize benzoxazinone synthesis9 (bx9) gene and maize enolpyruvylshikimate phosphate synthase1 gene.

Other embodiments provide, a method for increasing the expression of a native gene, comprising the following steps: (a) replacing in at least one cell of a plant the nucleic acid molecule of an intron; (b) introducing of a site-directed nuclease and a repair matrix into at least one cell of a plant, wherein the site-directed nuclease is able to generate at least two double strand breaks of the DNA in the genome in an intron of the native gene of the at least one cell and the repair matrix comprises the nucleic acid molecule of an intron or a fragment thereof; (c) cultivation of the at least one cell of (b) under conditions that allow a homology-directed repair or a homologous recombination, wherein the nucleic acid molecule is integrated from the repair matrix into the genome of the plant in the intron of the native gene; and (d) obtaining the plant having a replaced intron and the resulting increased expression of the native gene from the at least one cell. Other embodiments provide, a method for increasing the expression of a native gene, comprising the following steps: (a) replacing in at least one cell of a plant the nucleic acid molecule of an intron; (b) introducing of a site-directed nuclease and a repair matrix into at least one cell of a plant, wherein the site-directed nuclease is able to generate at least two double strand breaks of the DNA in the genome in an intron of the native gene of the at least one cell and the repair matrix comprises the nucleic acid molecule of an intron or a fragment thereof; (c) cultivation of the at least one cell of (b) under conditions that allow a homology-directed repair or a homologous recombination, wherein the nucleic acid molecule is integrated from the repair matrix into the genome of the plant in the intron of the native gene; and (d) obtaining the plant having a replaced intron and the resulting increased expression of the native gene from the at least one cell, wherein the intron is selected from the group of SEQ ID NO. 82-92.

Other embodiments provide, a method for increasing the expression of a native gene, comprising the following steps: (a) replacing in at least one cell of a plant the nucleic acid molecule of an intron; (b) introducing of a site-directed nuclease and a repair matrix into at least one cell of a plant, wherein the site-directed nuclease is able to generate at least two double strand breaks of the DNA in the genome in an intron of the native gene of the at least one cell and the repair matrix comprises the nucleic acid molecule of an intron or a fragment thereof; (c) cultivation of the at least one cell of (b) under conditions that allow a homology-directed repair or a homologous recombination, wherein the nucleic acid molecule is integrated from the repair matrix into the genome of the plant in the intron of the native gene; and (d) obtaining the plant having a replaced intron and the resulting increased expression of the native gene from the at least one cell, wherein the native gene is selected from the group of the maize benzoxazinone synthesis9 (bx9) gene and maize enolpyruvylshikimate phosphate synthase1 gene.

Other embodiments provide, a method for increasing the expression of a native gene, comprising the following steps: (a) replacing in at least one cell of a plant the nucleic acid molecule of a terminator; (b) introducing of a site-directed nuclease and a repair matrix into at least one cell of a plant, wherein the site-directed nuclease is able to generate at least two double strand break of the DNA in the genome in of an intron of the native gene of the at least one cell and the repair matrix comprises the nucleic acid molecule an intron or a fragment thereof; (c) cultivation of the at least one cell of (b) under conditions that allow a homology-directed repair or a homologous recombination, wherein the nucleic acid molecule is integrated from the repair matrix into the genome of the plant in the terminator of the native gene; and (d) obtaining the plant having a replaced terminator and the resulting increased expression of the native gene from the at least one cell.

Other embodiments provide, a method for increasing the expression of a native gene, comprising the following steps: (a) replacing in at least one cell of a plant the nucleic acid molecule of a terminator; (b) introducing of a site-directed nuclease and a repair matrix into at least one cell of a plant, wherein the site-directed nuclease is able to generate at least two double strand break of the DNA in the genome in of an intron of the native gene of the at least one cell and the repair matrix comprises the nucleic acid molecule an intron or a fragment thereof; (c) cultivation of the at least one cell of (b) under conditions that allow a homology-directed repair or a homologous recombination, wherein the nucleic acid molecule is integrated from the repair matrix into the genome of the plant in the terminator of the native gene; and (d) obtaining the plant having a replaced terminator and the resulting increased expression of the native gene from the at least one cell, wherein the intron is selected from the group of SEQ ID NO. 93-101.

Other embodiments provide, a method for increasing the expression of a native gene, comprising the following steps: (a) replacing in at least one cell of a plant the nucleic acid molecule of a terminator; (b) introducing of a site-directed nuclease and a repair matrix into at least one cell of a plant, wherein the site-directed nuclease is able to generate at least two double strand break of the DNA in the genome in of an intron of the native gene of the at least one cell and the repair matrix comprises the nucleic acid molecule an intron or a fragment thereof; (c) cultivation of the at least one cell of (b) under conditions that allow a homology-directed repair or a homologous recombination, wherein the nucleic acid molecule is integrated from the repair matrix into the genome of the plant in the terminator of the native gene; and (d) obtaining the plant having a replaced terminator and the resulting increased expression of the native gene from the at least one, wherein the native gene is selected from the group of the maize benzoxazinone synthesis9 (bx9) gene and maize enolpyruvylshikimate phosphate synthase1 gene.

Hereinafter, the present invention will be described in detail by the following examples. However, the following examples are illustrative of the present invention, and the scope of the present invention is not limited by the following examples.

### EXAMPLES

### Example 1: Identification of Novel Enhancer Sequences

Enhancers were selected from both maize and plant viruses for evaluation in the study. A report by Oka, et al. (2017) describing the identification of enhancer candidates in maize was mined for potential elements. In the report, data was obtained from two maize tissues, namely husk leaves and V2 stage inner stem tissue. The data generated included ChIPSeq data obtained from antibodies to H3K9ac (shown to be a mark of active enhancers in plants), DNAse-seq data, methylation data from bisulfate sequencing, and RNAseq data to profile gene expression in the two tissues. Targets were identified as genes 5 kb upstream or downstream of the candidate enhancers where a single gene in closest proximity in either direction was nominated as the target for a given enhancer. Genic and intergenic regions of the genome were categorized to distinguish transcriptional enhancer elements (“enhancers”) from promoters, exons, introns (including putative enhancer elements present within introns), flanking and distal intergenic regions, and terminators. Enhancers in close proximity to genes more highly expressed in either tissue were considered tissue specific; genes that were expressed significantly but not statistically different were considered to be constitutively expressed. The present disclosure focused on candidate enhancer sequences that were considered to be constitutively expressed, as they were deemed to be potentially more valuable to Insect Control trait gene expression. 221 candidate enhancer elements identified upstream of annotated genes were examined further. The genes were all named using accession IDs from the publicly available Maize genome, specifically, genome version B73 RefGen_v4, AGPv4) sourced from the website: https://www.maizegdb.org/. Gene expression profiles of all the genes found downstream of the constitutive enhancer candidates were examined using over 3500 samples (corresponding to 80+ tissue types) found in an internal installation of Genevestigator. The internal installation of Genevestigator included both publicly available data as well as proprietary Syngenta data. Data were first sorted by developmental stage to find genes expressed moderately to highly in most developmental stages (e.g., genes in the top quartile of all gene expression values across all samples in the local Genevestigator installation). Next, a subset of expressed genes was examined across tissue types. Data were also obtained for the ubiquitin gene Zm00001d015327 as a reference comparison. None of the assessed genes coupled downstream of the tested enhancer candidates had expression levels as high as ubiquitin, nor expressed as strongly in as many tissues as ubiquitin.

A second study was conducted to identify introns that might help drive and stabilize transgene expression. As part of that study, genes were ranked by low variance and high expression across 63 tissues from a Syngenta maize inbred line, an internal study, and by high protein expression from a subset of the same tissue samples. A list of the top 100 candidate genes was used. In total, 22 enhancer candidates were identified, including one downstream of a gene highly expressed in green tissue, one upstream of a gene highly expressed with low variance in 63 tissues from the Syngenta maize inbred line, one with high expression is silk tissue, and 19 genes with moderate to high expression in 75 tissues.

Sequences of viral genomes that contain promoters were selected for enhancer discovery. Experimentally defined enhancer elements from plant viral promoters have been identified in sequences within a few hundred nucleotides upstream of the conserved TATA box upstream of the transcriptional start site. The viral genomes examined were Chlorella virus (CviBIII, Mitra and Higgens (1994), Cestrum yellow leaf curling virus (CmYLCV, Stavolone et al 2003), Figwort Mosaic Virus (FMV Richins et al 1987), Cassava vein mosaic virus (CsVMV, Verdaguer et al 1996), Mirabilis mosaic virus (MiMV, Dey and Maiti 1999), Peanut chlorotic streak virus (PCISV, Maiti and Shepherd (1998), Rice Tungro bacilliform virus (RTBV, Hay et al 1991), and two strains of sugar cane bacilliform virus (ScBV Bouhida et al 1993; ScBv IM, Davies et al 2014), Blueberry Ringspot Virus (BRRV; Glasheen, et al 2002), Carnation etched ring virus (CERV; Hull et al 1986), Commelina Yellow Mottle Virus (CoYMV; Medberry, et al 1992), Dahlia Common Mosaic Virus (DCMV, Banerjee, 2015), Maize Streak Virus (MSV, Martin et al), Milk Vetch Dwarf Virus (MVDV, Shirasawa-Seo, et al 2005). The software tool NSITE (Shahmuradov I., Solovyev V. (2015) Nsite, NsiteH and NsiteM Computer Tools for Studying Transcription Regulatory Elements. Bioinformatics, doi: 10.1093ref) was used to identify potential regulatory elements (RE) within the sequences using default parameters. A total of 26 potential enhancer elements were identified from the viral sources.

### Example 2: Construction of Recombinant DNA Expression Cassettes Comprising Chimeric Enhancer Sequences

Recombinant DNA expression cassettes comprising one or more enhancer sequences, including the novel enhancer sequences described above were constructed using a combination of methods well known to those skilled in the art such as overlap PCR, DNA synthesis, restriction fragment sub-cloning and ligation. Multiple expression cassettes were constructed wherein each expression cassette comprised multiple operably linked components. The operably linked components included an enhancer element either in the form for of an individual enhancer (plant or viral, novel or known), or in the form of chimeric enhancers (herein provided as dual enhancers). The chimeric enhancers comprised a first plant enhancer contiguous to a second viral enhancer with the plant enhancer positioned upstream of the viral enhancer in some expression cassettes, and the viral enhancer positioned upstream of the plant enhancer in other expression cassettes. The individual or chimeric enhancer was operably linked to a plant promoter, which was operably linked to a heterologous polynucleotide transcribable by the promoter. In some constructs, the plant promoter was a constitutive plant promoter. In other constructs, the plant promoter was a tissue-specific plant promoter configured to drive expression of the operably linked to a heterologous polynucleotide in a tissue-specific manner. The cassette further comprised a terminator. As a result of the operably linked components, it was expected that transcription of the polynucleotide by the promoter would be increased by the chimeric enhancer in the expression cassette.

Control constructs were also created comprising chimeric enhancers having, for example, a first and second viral enhancer, a first and a second plant enhancer, or a first known plant enhancer coupled to a known viral enhancer, wherein the known plant enhancer and the known viral enhancer were enhancers whose expression profile from the given promoter was previously studied and known.

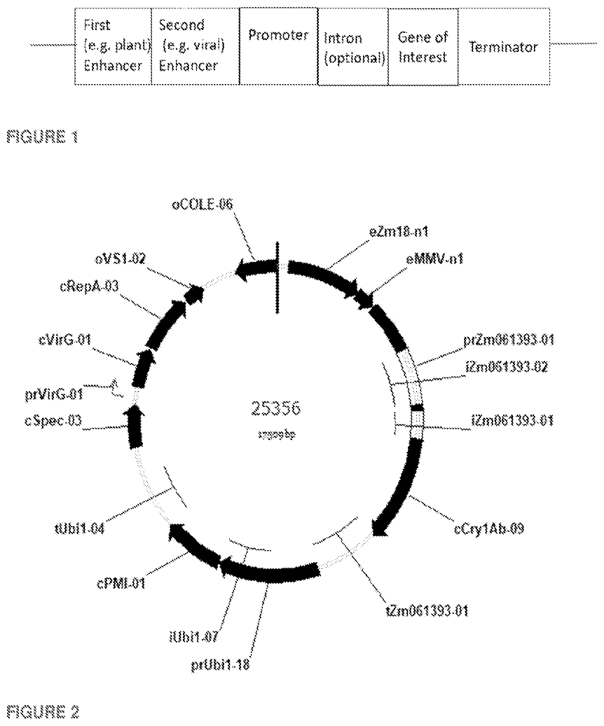

FIG. 1 shows an example design of a generic construct comprising the chimeric enhancer elements of the present disclosure. The example constructs comprise the chimeric enhancers of the present disclosure in an expression cassette operably linked to a promoter (which, in one example, as disclosed in Figure. 2, is a plant promoter, such as Maize Ubi1 promoter or Maize 393 promoter). The construct further comprises, operably linked to the chimeric enhancer, an optional intron, a gene of interest (such as a maize optimized truncated version of Cry1Ab), and a terminator (for example Nos promoter in Figure. 2). It will be appreciated that while the chimeric enhancer of FIG. 1 shows a plant enhancer element upstream of a viral enhancer element, this arrangement is not meant to be limiting and that in other constructs, the viral enhancer may be positioned upstream of the plant enhancer without departing from the scope of the invention.

Constructs generated using plant viral enhancers were pre-screened in a corn leaf transient assay (as described below) to determine their level of expression (data shown in Table 1). Those enhancers which improved expression levels in the pre-screening were selected to be further evaluated in combination with the newly identified plant transcriptional enhancers. The selection of plant transcriptional enhancers was random, but preferentially selected those that were smaller in length (<1 Kb).

Vectors were designed for individual or double candidate enhancer sequences, each of which contained a maize ubiquitin-1 promoter driving a truncated, maize optimized Cry1Ab gene, and the Nos terminator sequence. The truncated insect control gene Cry1Ab was used to evaluate all the enhancer elements of the present disclosure. For cloning convenience, enhancer sequences were synthesized as fragments flanked with SanDI (Kfll) and Acc65I restriction sites; these restriction sites were removed from other elements within the construct if present. Components of the double or chimeric enhancers were separated by a Xhol restriction site inserted between individual enhancers for cloning purposes.

The unique structures of the constructs are made explicit through the example of vector 25356 shown in FIG. 2.

It will be appreciated that while the constructs described above contain a maize ubiquitin-1 promoter, this is not meant to be limiting, and that similar constructs having similar or higher expression profiles may be generated using other promoters, such as other plant promoters as well as viral promoters, and other heterologous promoters. Likewise, while the constructs are used to describe the transcription enhancing effect of the chimeric enhancers on Cry1Ab, in other constructs, the chimeric enhancers may be similarly used to augment the transcription of an alternate gene of interest imparting a desired characteristic on a plant transformed with the construct.

### Example 3: Characterization of Constructs Comprising Chimeric Enhancer Sequences

### Methods

Vectors comprising the enhancer sequences were initially screened by transient transformation. Expression levels of all vectors were compared to average expression levels of a reference vector comprising a baseline positive control dual viral enhancer (eFMV+e35S, SEQ ID NO: 81). All vectors containing enhancer sequences were transformed into Agrobacterium tumefaciens strain EHA101 using Multi-well Electroporation 96 well format (www.harvardapparatus.com). Transformed Agrobacterium was grown for the in-planta corn leaf transient assay as described by Azhakanandam et al., (2007). Transient transformation was performed using 9-day old maize plants. Plants were cut off above the second node and damaged using a wire brush. Three plants were infiltrated per test construct. One milliliter of Agrobacterium suspension containing the test construct in addition to an internal control construct at a 1:1 ratio was pipetted on the top of the damaged stem tissue. Each Agrobacterium was adjusted to OD=1.0 at 600 nm. The infected plants were placed into a growth chamber for 3-4 days after which the infiltrated leaves were harvested in a 96 well block for a quantitative sandwich ELISA assay. A maximum of 8 data points was collected per construct based on the available tissue. The ELISA employed a monoclonal and a polyclonal antibody which had been produced against the Cry1Ab protein. High-binding polystyrene plates (Nunc Maxisorp #430341) were coated at 4° C. overnight with 1 ug/ml anti-Cry1Ab MAb in 25 mM borate, 75 mM NaCl, pH 8.5. Plates were washed five times with Phosphate Buffered Saline+0.05% Tween-20 (PBST). Standards (80, 40, 20, 10, 5, 2.5, 1.25 and 0 ng/ml of purified Cry1Ab protein) were prepared in ELISA diluent. One hundred microliters of each appropriately diluted sample or standard was added to the plate, incubated for 1 hr at ambient temperature with shaking at 200 rpm, and washed five times. Rabbit anti-Cry1Ab serum (100 ul/well) diluted 1/50,000 in ELISA diluent was then added to the plate, incubated for 1 hour at ambient temperature with shaking at 200 rpm, and washed as before. Donkey anti-rabbit serum conjugated to alkaline phosphatase (Jackson ImmunoResearch, West Grove, PA) at 1 ug/ml in ELISA diluent was added to the plate (100 ul/well), incubated at ambient temperature with shaking at 200 rpm, and washed. Substrate p-nitrophenyl phosphate (Surmodics, Eden Prairie, MN) was added and allowed to develop for 15-30 min at ambient temperature. The absorbance was measured at 405 nm using a microplate reader (BioTek Powerwave XS2, Winooski, VT). The standard curve used a four-parameter curve fit to plot the concentrations versus the absorbance. To normalize for extraction efficiency, the concentration of the analyte (Cry1Ab) was divided by the concentration of the total soluble protein (TSP). TSP was measured using the Pierce™ BCA (bicinchoninic acid) protein assay (ThermoFisher Scientific).

### Results

80 vectors correspondingly comprising 80 different enhancer sequences were tested in a transient transformation assay. Of these 80 vectors, 48 vectors contained single enhancers (SEQ ID NOs: 1-48) and 32 contained dual enhancers (SEQ ID NOS: 49-80). Of the 80 tested vectors, 23 were found to have expression levels comparable to or higher than the positive control (eFMV+e35S, SEQ ID NO: 81) comprising dual enhancers (Table 1). The majority of tested expression cassettes exhibiting expression levels higher than the positive control were those containing dual enhancers. Three viral enhancers eMMV (SEQ ID NO: 30), eCsVMV (SEQ ID NO: 26), and ePCISV (SEQ ID NO: 32), were present in dual enhancer combinations in most of the constructs exhibiting highest expression levels.

Expression cassettes were ranked based on their expression levels relative to the baseline positive cassette. The top performing cassettes were selected for further experiments using stable transformants (Table 2). As seen in the table, the dual transcriptional enhancer combination of plant enhancer eZm18 and viral enhancer eMMV (construct 25356) more than doubled the expression level of truncated Cry1Ab in leaf tissue relative to the positive control cassette (construct 25078). This result is unexpected due to the lower expression levels observed with the corresponding individual plant and viral enhancers (constructs 25370 and 25491). Additionally, expression cassettes comprising chimeric enhancers having other combinations of plant and viral enhancers, such as constructs with the combination of plant enhancer eZm19 and viral enhancer ePCISV (construct 25361), and plant enhancer eZm18-n1 and viral enhancer eMMV-n1 (construct 25356), exhibited expression levels which exceeded that of the dual enhancer positive control construct (25078).

TABLE 1
Enhancer transient transformation expression assay results

| | Enhancer | | |
|---|---|---|---|
| | SEQ ID | Avg | % of |
| Enhancer Name | NO | Cry1Ab/CFP | 25078 |
| eZm00001d052734 (eZm18): eMMV | 77 | 189.1 | 195% |
| eCmYLCV: ePCISV | 58 | 179.1 | 185% |
| eMMV: eScBV | 56 | 166.2 | 172% |
| eZm00001d002338 (eZm1): eMMV | 67 | 161.7 | 167% |
| ePCISV: eCsVMV | 62 | 158.7 | 164% |
| eMMV: eCsVMV | 57 | 158.1 | 163% |
| eCsVMV: eMMV | 52 | 157.2 | 162% |
| eZm00001d053090 | 79 | 153.7 | 159% |
| (eZm19): ePCISV | | | |
| eZm00001d015463 | 78 | 148.3 | 153% |
| (eZm22): eCmYLCV | | | |
| eCsVMV | 26 | 142.2 | 147% |
| eCmYLCV: eScBV | 59 | 133.3 | 138% |
| eCvi: eCsVMV | 54 | 130.7 | 135% |
| eZm00001d041480 | 75 | 125.1 | 129% |
| (eZm14): eCsVMV | | | |
| ePCISV: eScBV | 61 | 125.1 | 129% |
| eCsVMV: eScBV | 51 | 125.0 | 129% |
| eFMVsg: ePCISV | 64 | 123.7 | 128% |
| eZm00001d005179 (eZm3): eMMV | 68 | 123.6 | 128% |
| ePCISV | 32 | 121.3 | 125% |
| eZm00001d030618 (eZm8): eScBV | 70 | 112.2 | 116% |
| ePCISV: eCvi | 63 | 111.6 | 115% |
| eFMV | 28 | 106.6 | 110% |
| eMMV: eNOS | 55 | 102.6 | 106% |
| eZm00001d002338 | 65 | 98.4 | 102% |
| (eZm1): eCsVMV | | | |
| eFMV: e35s (positive control) | | 165.1 | 100% |
| eFMV: e35s (positive control) | | 96.7 | 100% |
| eCmYLCV: eCvi | 60 | 95.6 | 99% |
| eCsVMV: eNOS | 50 | 91.4 | 95% |
| eZm00001d017850 (eZm5): eScBV | 72 | 89.0 | 92% |
| eZm00001d050403 (eZm21): eScBV | 80 | 84.1 | 87% |
| eScBV | 34 | 83.2 | 86% |
| eCsVMV: eZm00001d002338 | 66 | 77.0 | 80% |
| (eZm1) | | | |
| eMMV | 30 | 76.6 | 79% |
| eScBV: eZm00001d030618 (eZm8) | 71 | 75.6 | 78% |
| e35S: eFMV | 49 | 70.1 | 72% |
| eCmYLCV | 31 | 69.1 | 71% |
| eScBVIM | 35 | 65.4 | 68% |
| eMMV: eZm00001d005179 (eZm3) | 69 | 64.2 | 66% |
| e35S | 25 | 56.7 | 59% |
| eFMV | 24 | 28.9 | 30% |
| eZm00001d035657(eZm11): eFMV | 74 | 23.1 | 24% |
| eFMVsg | 29 | 17.9 | 18% |
| eZm00001d035657 (eZm11) | 11 | 28.3 | 17% |
| eMDV | 48 | 27.8 | 17% |
| eCERV-L | 38 | 24.9 | 15% |
| eNOS | 23 | 14.3 | 15% |
| eDCMV_p2 | 40 | 22.9 | 14% |
| eZm00001d035693 (eZm12) | 12 | 19.5 | 12% |
| eZm00001d005179 (eZm3) | 3 | 17.8 | 11% |
| 25189 baseline without enhancers | | 11.3 | 7% |
| eCoYMV | 39 | 9.4 | 6% |
| eZm00001d034223 (eZm9) | 9 | 7.1 | 4% |
| eZm15: eCvi | 76 | 4.1 | 4% |
| eZm00001d045635 (eZm15) | 15 | 5.2 | 3% |
| eCvi: eNOS | 53 | 2.9 | 3% |
| eZm00001d030618 (eZm8) | 8 | 4.3 | 3% |
| eMSV6 | 46 | 4.1 | 2% |
| eMSV3 | 43 | 3.9 | 2% |
| eCERV-s | 37 | 3.8 | 2% |
| eMSV5 | 45 | 2.4 | 1% |
| eRTBV | 33 | 1.3 | 1% |
| eMSV7 | 47 | 2.2 | 1% |
| eZm00001d004841 (eZm2) | 2 | 2.1 | 1% |
| eZm00001d015463 (eZm22) | 22 | 1.8 | 1% |
| eCvi | 27 | 1.0 | 1% |
| eMSV1 | 41 | 1.6 | 1% |
| eZm7: eNOS | 73 | 0.8 | 1% |
| eZm00001d017850 (eZm5) | 5 | 1.3 | 1% |
| eMSV4 | 44 | 1.1 | 1% |
| eZm00001d002338 (eZm1) | 1 | 0.0 | 0% |
| eZm00001d005798 (eZm4) | 4 | 0.0 | 0% |
| eZm00001d020804 (eZm6) | 6 | 0.0 | 0% |
| eZm00001d029426 (eZm7) | 7 | 0.0 | 0% |
| eZm00001d034223 (eZm10) | 10 | 0.0 | 0% |
| eZm00001d039526 (eZm13) | 13 | 0.0 | 0% |
| eZm00001d041480 (eZm14) | 14 | 0.0 | 0% |
| eZm00001d046472 (eZm16) | 16 | 0.0 | 0% |
| eZm00001d048707 (eZm17) | 17 | 0.0 | 0% |
| eZm00001d052734 (eZm18) | 18 | 0.0 | 0% |
| eZm00001d053090 (eZm19) | 19 | 0.0 | 0% |
| eMSV8 | 20 | 0.0 | 0% |
| eZm00001d050403 (eZm21) | 21 | 0.0 | 0% |
| eBBRV | 36 | 0.0 | 0% |
| eMSV2 | 42 | 0.0 | 0% |

TABLE 2
Enhancer stable transformation assay results (T0 events, V2 stage leaf tissue)

| | | | | | | X-fold |
|---|---|---|---|---|---|---|
| | | | | | | increase |
| | | # of | | | | over |
| | | single | | | | enhancer- |
| Construct | | copy | Cry1Ab ng/mg (TSP) | | | less |
| ID | Cassette | events | Low- High | Average | Median | construct |
| 25356 | 1. eZm18-n1, eMMV-n1, | 10 | 526-967 | 792 | 781 | 205 |
| | prZm061393-01, cCry1Ab-09, | | | | | |
| | tZm061393-01 | | | | | |
| 25365 | ePCISV-n1, prZm061393-01, | 10 | 235-714 | 541 | 498 | 131 |
| | cCry1Ab-09, tZm061393-01 | | | | | |
| 25361 | eZm19-n1, ePCISV-n1, | 10 | 203-470 | 328 | 354 | 93 |
| | prZm061393-01, cCry1Ab-09, | | | | | |
| | tZm061393-01 | | | | | |
| 25370 | eMMV-n1, prZm061393-01, | 10 | 137-464 | 342 | 326 | 86 |
| | cCry1Ab-09, tZm061393-01 | | | | | |
| 25362 | eCsVMV-n1, eMMV-n1, | 10 | 144-574 | 351 | 312 | 82 |
| | prZm061393-01, cCry1Ab-09, | | | | | |
| | tZm061393-01 | | | | | |
| 25078 | eFMV-06, e35S-11, prZm061393- | 7 | 4-471 | 224 | 284 | 75 |
| (positive | 01, cCry1Ab-09, tZm061393-01 | | | | | |
| control) | | | | | | |
| 25368 | eZm8-n1, eScBV-n2, prZm061393- | 10 | 94-654 | 287 | 259 | 68 |
| | 01, cCry1Ab-09, tZm061393-01 | | | | | |
| 25369 | eScBV-n2, prZm061393-01, | 7 | 146-1442 | 412 | 248 | 65 |
| | cCry1Ab-09, tZm061393-01 | | | | | |
| 25366 | eCsVMV-n1, prZm061393-01, | 8 | 127-231 | 179 | 173 | 46 |
| | cCry1Ab-09, tZm061393-01 | | | | | |
| 25367 | eFMV-06, prZm061393-01, | 10 | 63-151 | 101 | 99 | 26 |
| | cCry1Ab-09, tZm061393-01 | | | | | |
| 25491 | eZm18-n2, prZm061393-01, | 14 | 2.4-18 | 6 | 4.4 | 1.1 |
| | cCry1Ab-09, tZm061393-01 | | | | | |
| 25189 | prZm061393-01, cCry1Ab-09, | 10 | 1.4-7.7 | 3.6 | 3.8 | 1 |
| (base | tZm061393-01 | | | | | |
| promoter | | | | | | |
| with no | | | | | | |
| enhancers) | | | | | | |

### Example 4: Characterization of Tissue-Specific Expression of Constructs Comprising Chimeric Enhancer Sequences

Expression of trCry1Ab was evaluated for tissue specificity in the events comprising expression cassettes described in Example 3. The addition of enhancers and combinations of enhances increased the expression level over that seen with the promoter alone. High levels of expression are desirable for insect control genes. Particularly, high expression in root is beneficial for expression of corn rootworm traits. Additional samples were collected at V8 stage from tissue types including root, silk, husk, pollen, and kernel and assayed via ELISA (Table 3). Surprisingly, the constructs with chimeric enhancers maintained low expression of trCry1Ab in pollen, despite higher expression in other tissues such as leaf, root, and husk. In the majority of constructs the expression levels in pollen were lower than that of construct 25189 with no enhancer element.

TABLE 3
Tissue specific expression analysis for maize
events comprising enhancer expression cassettes

| | | An average Cry1Ab ng/mg (TSP) in T0 single copy events | | | | | |
|---|---|---|---|---|---|---|---|
| Construct | | Leaf | Root | Silk | Husk | Pollen | Kernel |
| ID | Enhancers | 10 | 8 | 10 | 10 | 9 | 20 |
| 25189 | No enhancer | 3.61 | 37.10 | 1.51 | 23.48 | 1.52 | 907.65 |
| 25078 | eFMV + 35S | 224.79 | 562.21 | 75.50 | 393.04 | 0.53 | 177.45 |
| 25356 | eZm18 + eMMV | 792.98 | 754.46 | 374.55 | 902.78 | 1.39 | 355.44 |
| 25361 | eZm19 + ePCISV | 328.94 | 1142.31 | 221.51 | 646.17 | 0.88 | 666.97 |
| 25368 | eZm8 + eScBV | 287.98 | 660.32 | 67.21 | 486.41 | 5.57 | 139.51 |
| 25367 | eFMV-06 | 101.08 | 162.76 | 46.43 | 220.96 | 0.51 | 1208.04 |
| 25370 | eMMV | 342.92 | 771.21 | 335.08 | 558.39 | 0.98 | 338.58 |
| 25365 | ePCISV | 541.21 | 1151.53 | 366.84 | 531.14 | 1.24 | 429.32 |
| 25369 | eScBV | 412.73 | 1623.68 | 158.96 | 1283.01 | 27.04 | 2312.07 |
| 25368 | eZm8 | 289 | 660 | 67 | 86 | 5.5 | 139 |
| 25489 | eZm11 | 6 | 73 | 7 | 37 | 8.8 | 153 |
| 25490 | eZm12 | 7.3 | 74 | NA | 29 | 23 | 161 |
| 25491 | eZm18 | 6.3 | 35 | 4.2 | 34 | 1.8 | 221 |
| 25492 | eZm19 | 5 | 38 | 4.6 | 23 | 8.5 | 247 |

### Example 5: Identification and Characterization of Novel Intron Sequences

### Methods

Candidate intron sequences were selected based on potential to enhance gene expression. First, genes with relatively high stable expression across various tissues and conditions were selected from available transcript and proteomics data. Transcript level data was analyzed to identify gene isoforms with high, stable expression across all samples tested (data not shown). Genes with high transcript expression were then filtered using maize proteomics data from multiple tissues. The top 50 most abundant proteins were selected. The first introns for 44 of the genes were analyzed using IMEter v2.1 (http://korflab.ucdavis.edu/cgi-bin/IMEter_2014/web-imeter2.1.pl). Ten introns were selected based on high transcript, high protein, and highest IMEter scores. Introns iBdUbi10, iBdEF1a (Coussens, et al 2012) and iPvUbi1 (Mann, et al 2011) were selected based on strong constitutive activities of their corresponding promoters and the prediction that they would have the same enhancing capabilities as iUbi1 (positive control). Intron sequences were annotated from their promoter sequences provided by reference papers. iZmABP3, iZm061393, and iZm009722 were identified from internally tested plant promoters, with annotation checked/confirmed by Persephone. iZmHSP70 was identified based on its use in the art for multiple events (Mann et al 2011). iUbi1-V31 is a truncated version of the positive control iUbi1 that displayed an enhanced effect. Both introns iZmGRMZM2G149768 and iZmGRMZM2G088088 were identified using bioinformatics.

Vectors were designed for individual intron sequences, each of which contained a maize ubiquitin-1 promoter driving a truncated, maize optimized Cry1Ab gene, and the Nos terminator sequence. The truncated insect control gene Cry1Ab was used to evaluate all the elements in the invention. Intron sequences, often with short flanking promoter sequences on both ends to ensure proper splicing, were synthesized as fragments flanked with Avrll and Ncol sites for cloning convenience. These restriction sites were removed from other tested elements within the construct if present.

The vectors were then stably transformed into maize plants, validated at TO events, and compared to control events. A. tumefaciens strain LBA4404 (pAL4404, pVGW7) was used for maize transformation. Detailed information about the pAL4404 and pVGW7 helper plasmid and the virulence region is described by Imayama et al (U.S. Pat. No. 10,266,835), Ishida et al. (Nat. Biotechnol., 1996, 14:745-750) and Negrotto et al. (Plant Cell Rep., 2000, 19:798-803). A. tumefaciens strains containing the binary vectors containing all test constructs were prepared as described by Li et al. (Plant Physiol., 2003, 133:736-47). For maize transformation, immature embryos from greenhouse grown maize inbred line NP2222 were used as explants (Zhong, et al., 2018, Methods Mol. Biol., 1676:41-59). Immature embryo isolation, Agrobacterium inoculation and co-cultivation of Agrobacterium with the immature embryos were performed as described by Li et al. (reference incorporated above) with modifications (Sivamani, et al., 2019, Mol. Biol. Rep., 46:3009-3017). Transformed tissues and putative transgenic events were generated on media using mannose selection as described earlier.

Cultivation of maize inbred NP2222 occurred in the greenhouse to generate immature embryos for transformation. Seeds were sown in pots containing Fafard® Redi-Earth media. Two-week old seedlings are transplanted to 3-gallon pots with Fafard® 3 media. Stock plants were grown in 14-hour photoperiod at 27° C. day/21° C. night and irrigated systematically with fertilizer water. Emerged ear shoots were covered with pollination bags to prevent contamination. Controlled pollinations were carried out manually and immature ears with developing kernels were harvested at 9-10 days after pollination for immature embryo extraction.

Events were generated through transformation and returned to the greenhouse when rooted in an agar media. Plantlets were transferred to pots with Fafard® Redi-Earth media for 10 to 14 days. Plantlets were sampled for Taqman copy number analysis and ELISA analysis (Example 1) prior to transplantation. Specifically, real-time PCR was set up in 384-well plates. Reactions were multiplexed to simultaneously amplify the target gene and endogenous control gene. For each sample, the Taqman Assay was setup by combining 3 μl of extracted genomic DNA with 3 μl master mix containing Jumpstart Taq ReadyMix (Sigma) supplemented with primers to a final concentration of 300 nM each and probes to a final concentration of 100 nM each. The 384-well plates were heat sealed, and the real-time PCR was carried out in either the ABI 7900 Real-time PCR machine or the Life Technologies Quant Studio Flex 7 instrument, using the following parameters: 95° C. for 5 minutes, 40 cycles of 95° C. for 5 seconds and 60° C. for 30 seconds. Post-run data analysis was performed according to the manufacturer's instructions. Single copy events were transplanted to 3-gallon pots with Fafard@ 3 media, maintained in 14-hour photoperiod at 27° C. day/21° C. night and irrigated systematically with fertilizer water. During the vegetative life cycle, leaf tissue and reproductive tissues were collected at various timepoints for ELISA protein expression analysis as described in Example 1. During reproductive stage, silk and pollen were collected and stored at −80° C. until sample plates were prepared for lab delivery. Events were self-pollinated and/or outcrossed to a tester inbred to create T1/F1 hybrids for further expression analysis.

### Results

10 vectors containing 10 different candidate introns were tested in stable maize transformants. The data revealed that the intron-mediated enhancement is more prominent for some introns than others. For example, iBdUbi10-v2 enhanced expression 4× more compared with iZmGRMZM2G088088. Of those 10 introns tested, 4 expression cassettes were found to have expression levels of truncated Cry1Ab that were comparable or better in stable transformants than the cassette containing positive control intron iUbi1-30, which is one of the strongest enhancing introns as currently reported in the literature (SEQ ID NO: 92) (Table 4).

TABLE 4
Expression of reporter, cCry1Ab-09, in leaf tissue

| | | Average |
|---|---|---|
| | | ELISA |
| Construct | | (ng/mg |
| ID | Cassette details | TSP) |
| 24832 | prUbi1-44, iBdUbi10-v2, cCry1Ab-09, tNOS-05-01 | 568 |
| 24833 | prUbi1-44, iBdEF1a-v1, cCry1Ab-09, tNOS-05-01 | 337 |
| 24835 | prUbi1-44, iUbi1-v31, cCry1Ab-09, tNOS-05-01 | 312 |
| 24838 | prUbi1-44, iPvUbi1, cCry1Ab-09, tNOS-05-01 | 302 |
| 24310 | prUbi1-44, iUbi1-30, cCry1Ab-09, tNOS-05-01 | 277 |
| (Positive | | |
| control) | | |
| 24834 | prUbi1-44, iZmABP3-02, cCry1Ab-09, tNOS-05-01 | 220 |
| 24837 | prUbi1-44, iZmGRMZM2G149768, cCry1Ab-09, tNOS-05-01 | 197 |
| 24839 | prUbi1-44, iZm061393-v3, cCry1Ab-09, tNOS-05-01 | 136 |
| 24836 | prUbi1-44, iZm009722-01, cCry1Ab-09, tNOS-05-01 | 128 |
| 24846 | prUbi1-44, iZmHSP70-V2, cCry1Ab-09, tNOS-05-01 | 127 |
| 24840 | prUbi1-44, iZmGRMZM2G088088, cCry1Ab-09, tNOS-05-01 | 112 |

### Example 6: Identification and Characterization of Novel Terminator Sequences

### Methods

Candidate terminator sequences were selected from internal data as well as from literature which were used for overexpression of different proteins in monocots. tSoUbi4 (Wei et al 2003), tBdEF1a (Coussens et al 2012), tOsAct1 (McElroy et al 1990), tOsRubiq2 (Wang et al 2000), and tPvUbi1 (Mann et al 2011) were selected based on strong constitutive expression of their associated genes and the assumption that they contributed to the expression levels. The sequences were selected based on annotation of their corresponding gene models in their specific genomes. tZmUGTBx9 and tZmABP1 were also selected based on their performance when evaluated with internally identified genes.

Vectors were designed for individual terminator sequences, each of which contained a maize ubiquitin-1 promoter driving a truncated, maize optimized Cry1Ab gene, and the unique terminator sequence. The truncated insect control gene Cry1Ab was used to evaluate all the elements in the invention. Terminator sequences were synthesized as fragments flanked with Sacl and Rsrll sites for cloning convenience; these restriction sites were removed from other tested elements within the construct if present.

The vectors were then stably transformed into maize plants, validated at TO events, and compared to control events. The transformation and analysis methods are the same as those described in Example 2.

### Results

8 vectors containing 8 different candidate terminators were tested in a stable maize transformants. The data revealed that the terminator-mediated enhancement is prominent for most of the terminators tested. Terminator-mediated enhancement was at a maximum 6-fold increase with tOsRubiq2. Of those 8, 6 expression cassettes were found to have expression levels of truncated Cry1Ab comparable or better in stable transformants than the cassette containing the positive control terminator tNos-05-01 (SEQ ID NO: 101) (Table 5).

TABLE 5
Expression of reporter, cCry1Ab-09, in leaf tissue

| | | Average T0 |
|---|---|---|
| | | expression |
| Construct | | ng/mg |
| ID | Cassette details | (TSP) |
| 24824 | prUbi1-44, cCry1Ab-09, | 842 |
| | tOsRubiq2 | |
| 24826 | prUbi1-44, cCry1Ab-09, | 779 |
| | tZmABP-01 | |
| 24827 | prUbi1-44, cCry1Ab-09, | 480 |
| | tZmUGTBx9 | |
| 24825 | prUbi1-44, cCry1Ab-09, tPvUbi1 | 404 |
| 24831 | prUbi1-44, cCry1Ab-09, | 368 |
| | tOsAct1-V1 | |
| 24830 | prUbi1-44, cCry1Ab-09, tUbi1- | 322 |
| | 04 | |
| 24310 | prUbi1-44, cCry1Ab-09, tNOS- | 277 |
| (positive | 05-01 | |
| control) | | |
| 24823 | prUbi1-44, cCry1Ab-09, tSoUbi4 | 242 |
| 24822 | prUbi1-44, cCry1Ab-09, tBdEF1a | 131 |

### Example 7: Characterization of Tissue-Specific Expression of Constructs Containing Novel Intron and Terminator Sequences

### Methods

Selected intron and terminator sequences shown can enhance leaf expression of prUbi1 were used to test improving expression of the tissue-specific promoter.

Vectors were designed for testing the promoter prZm061393, published in WO2021043942, with its leader intron swapped with iBdUbi10 or iPvUbi1, or paired with tOsAct1. Additional vectors were also designed to test combinations of stronger introns and terminators on prZm061393 compared with nature prZm061393-tZm061393 pair. A truncated, maize optimized Cry1Ab gene was used to evaluate all the combinations of elements in this experiment. Intron sequences, often with short flanking promoter sequences at 5′end and the rest of 3′end to ensure proper splicing, were synthesized as fragments flanked with Avrll and Ncol sites for cloning convenience. Terminator sequences were synthesized as fragments flanked with Sacl and Rsrll sites for cloning convenience; these restriction sites were removed from other tested elements within the construct if present.

The vectors were then stably transformed into maize plants, validated at TO events, and compared to control events. The transformation and analysis methods are the same as those described in Example 2.

### Results

Five vectors were tested in stable maize transformants (See Table 6). The data revealed that introns iBdUbi10 or iPvUbi1 can effectively enhance the activities or prZm061393 in multiple tissues, including leaf, root, silk, husk, and causing significant expression in pollen-tissues, different from when enhanced by transcriptional enhancers shown in Table 3. Differently, tested terminators did not exhibit as significant impact, to either expression levels or tissue-specificity.

TABLE 6
Tissue specific expression analysis for maize events comprising expression
cassettes with intron and terminator swapped with heterologous sequences.

| Construct | | An average Cry1Ab ng/mg (TSP) in T0 single copy events | | | | | |
|---|---|---|---|---|---|---|---|
| ID | Enhancers | Leaf | Root | Silk | Husk | Pollen | Kernel |
| 25189 | No enhancer | 3.61 | 37.10 | 1.51 | 23.48 | 1.52 | 907.65 |
| 25078 | eFMV + 35S | 224.79 | 562.21 | 75.50 | 393.04 | 0.53 | 177.45 |
| 25494 | iBdUbi10 | 43.14 | 144.7 | 51.1 | 266.86 | 90.48 | 402.21 |
| 25496 | iPvUbi1 | 29.84 | 139.58 | 56 | 104 | 101.58 | 552.2 |
| 25497 | tOsAct1 | 2.73 | 16.59 | ND | 16.18 | 6.8 | 178.24 |
| 25538 | iBdUbi10 + tSoUbi | 48.36 | 287.53 | 62.12 | 361.16 | 80.17 | 450.12 |
| 25539 | iPvUbi1 + tOsAct1 | 39.9 | 83.51 | 53.73 | 202.23 | 61.56 | 253.31 |

### Example 8: Characterization of Tissue-Specific Expression of a Root-Specific Promoter Enhanced by a Viral Enhancer eMMV

### Methods

Vectors were designed for testing the root-specific promoter prOsRcc3 described in patent application US2006101541 paired with its native terminator without or in the presence of the eMMV enhancer. A maize optimized FR8A gene was used to evaluate combinations of elements in this experiment.

The vectors were then stably transformed into maize plants, validated at TO events, and compared to control events. The transformation and analysis methods are the same as those described in Example 2.

### Results

Two vectors were tested in stable maize transformants (See Table 7). The data revealed that eMMV can effectively enhance this root-specific promoter without causing significant expression (>1/10 of root expression levels) in all other tested tissues.

TABLE 7
Tissue specific expression analysis for maize events with
and without eMMV enhancement on a root-specific promoter

| Construct | | An average Cry1Ab ng/mg (TSP) in T0 single copy events | | | | | |
|---|---|---|---|---|---|---|---|
| ID | Enhancers | Leaf | Root | Silk | Husk | Pollen | Kernel |
| 26155 | No enhancer | 2.24 | 54.51 | 0.96 | 0 | 0.04 | 0 |
| 26159 | eMMV | 5.48 | 435.08 | 1.95 | 6.84 | 0.02 | 0.15 |

### Example 9: Uses of Regulatory Elements for Native Genes

In another aspect, provided herein are methods of upregulating the expression of a native gene in maize or soybean that involves using genome editing (i.e. CRISPR) to stably insert an enhancer (for instance, viral or maize enhancers in this application) into the native gene, or to replace introns or terminators using introns or terminators (such as those in this application from same or different plant species) to boost the expression of that gene with stronger activities, or for desirable tissue-specificity. In some embodiments, the process will involve providing to a plant cell, via biolistic bombardment, a CRISPR-Cas12a or CRISPR-Cas9 protein enzyme in addition to a guide RNA targeted to a specific location in a genome of interest, along with donor DNA comprising one or more of the enhancers in this application. In another embodiment, the CRISPR enzyme and gRNA and donor DNA are provided in a plasmid to the plant cell via biolistic or agrobacterium-mediated transformation.

The target gene for upregulation may be an herbicide tolerance gene, a disease resistance or tolerance gene, a drought tolerance gene, a nutrient composition gene, a plant morphology gene, or a yield component gene, or any gene relevant to crop improvement. The target location for insertion of the enhancers could be in the promoter, the 5′ UTR, the first intron, the 3′ UTR, or the terminator, or any other part of the gene or surrounding gene regulatory sequence.

For example, the promoter region of the maize benzoxazinone synthesis9 (bx9) gene will be modified by CRISPR-Cas12a-mediated insertion of an enhancer. The Bx9 gene is Zm00001eb033030 at chromosome 1, position 184,390,111-184,394,952 in maize B73_NAM_v5 reference genome (or Zm00001d031209 in B73_v4 and GRMZM2G161335 in B73_v3). Cas12a ribonucleoprotein particles (RNP), complexed with a guide RNA with the target sequence 5′-GCGAGAGGGGCGTGGGACCCACG-3′ (SEQ ID NO. 108)(PAM site for this target sequence is TTTC) will be delivered to maize embryo or callus cells in the genetic background NP2222 in order to make a double stranded break at a specific site in the bx9 gene, 20 bp upstream of 5′UTR region. In addition to this machinery for making a double stranded break, donor DNA sequence comprising one of the enhancers will be provided to the same cells and tissues to enable insertion at the target site.

As another example, the promoter region, 5′UTR, first exon or intron of the maize enolpyruvylshikimate phosphate synthase1 gene (abbreviated as EPSPS or EPS1) gene (Zm00001d045450) will be targeted for CRISPR-mediated insertion of one of the enhancers listed in this application, in order to upregulate that gene to confer stronger tolerance to the herbicide glyphosate. Alternatively, one of the native introns or the native terminator will be replaced with one of the introns or terminators listed in this application, in order to strengthen the expression to confer stronger tolerance to glyphosate. In other embodiments, these enhancements which will strengthen the expression of the EPS1 or EPSPS gene will be combined with a modification to the coding sequence, changing an amino acid to confer glyphosate tolerance.

In some embodiments, the donor DNA will consist of the enhancer, intron, or terminator alone or it will be flanked by <30 bp primer or spacer sequences and the repair pathway mediating the insertion will be non-homologous end joining. In other embodiments, the DNA donor comprising the enhancer, intron or terminator will be flanked on either side by extended DNA sequences (>30 bp but <1500 bp) matching (or with high homology to) the upstream and downstream sequences flanking the target site. These sequences would be included to promote efficient insertion or sequence replacement mediated by the repair pathway called homology directed repair (HDR) (in this method the donor is recombined into the target site). For example, using 50 bp flanking homologous sequences, the DNA donor described above for insertion of an enhancer about 20 bp upstream of the 5′-UTR would comprise the following sequence, CTCCCTTCCCCCCACTAATAAATTACTAACACATCGTGGGTCCCACGCCCNNN enhancer sequence is put here NNNNCCCACGCCCCTCTGGGGTTACACGCACGCGCCGTCCTTTTCTCCCCTCTA (SEQ ID NO. 109). Another example would be to use the flanking sequence from the first and second exon of the EPSPS gene as homologous sequences that flank the donor DNA comprising the enhancing intron to be inserted. This way, via HDR, the native first intron is replaced with the exogenous “stronger” intron without affecting EPSPS splicing.

In some cases, the CRISPR Cas12a-crRNA RNP complex and donor DNA fragment (with or without the flanking template matching the target site) will be co-delivered to maize immature embryos through biolistic transformation. In other cases, the CRISPR and gRNA will be delivered via agrobacterium mediated transformation while the donor DNA will be delivered via biolistic transformation. In yet other embodiments, the CRISPR enzyme, guide and donor DNA will all be delivered via agrobacterium. After transformation, transgenic events will be recovered and analyzed for the targeted enhancer insertion.

A PCR assay will be run to establish which events have the insertion correctly inserted, and then the expression of the gene will be measured by qRT-PCR (RNA level) or ELISA (protein level) to detect the increased expression in any number of target tissues (e.g. embryo, callus, leaf, root, flower, pollen, stem, or other tissue). Events with the successful enhancer insertion and increased expression level will then be evaluated phenotypically for the improvement of the trait of interest.

1. (canceled)

2. (canceled)

3. An expression cassette comprising a chimeric transcriptional enhancer operably linked to a promoter sequence, wherein the chimeric transcriptional enhancer comprises a first nucleotide sequence operably linked to a second nucleotide sequence, wherein the first nucleotide sequence is a plant transcriptional enhancer and the second nucleotide sequence is a viral transcriptional enhancer.

4. The expression cassette of claim 3, wherein the operably linked nucleotide sequences are contiguous and wherein the chimeric transcriptional enhancer has at least 90% identity with or comprises one or more of SEQ ID NOS: 49 to 80.

5. The expression cassette claim 3, wherein the nucleotide sequence of the plant enhancer has at least 90% identity with or comprises one of SEQ ID NOs: 1 to 21.

6. The expression cassette of claim 3, wherein the nucleotide sequence of the viral transcriptional enhancer has at least 90% identity with or comprises one of SEQ ID NOs: 22 to 48.

7. The expression cassette of claim 5, wherein the plant enhancer is selected from the group consisting of SEQ ID NO: 18, SEQ ID NO: 19, SEQ ID NO: 8, SEQ ID NO: 11, and SEQ ID NO: 12.

8. The expression cassette of claim 5, wherein the viral enhancer is selected from the group consisting of SEQ ID NO: 30, SEQ ID NO: 32, SEQ ID NO: 26, and SEQ ID NO: 34.

9. The expression cassette of claim 1, wherein the chimeric transcriptional enhancer is operably linked to a heterologous nucleotide sequence.

10. The expression cassette of claim 1, wherein the heterologous nucleotide sequence is a nucleic acid of interest that encodes an RNA or protein of interest.

11. The expression cassette of claim 10, wherein the RNA or protein of interest is capable of conferring upon a plant a desired characteristic such as antibiotic resistance, virus resistance, insect resistance, disease resistance, resistance to other pests, herbicide tolerance, improved nutritional value, improved performance in an industrial process or altered reproductive capability.

12. The expression cassette of any of claim 1, wherein the heterologous nucleotide sequence encodes a selectable marker or wherein the expression cassette further comprises a selectable marker.

13. A vector comprising the expression cassette of claim 1.

14. The vector of claim 13, wherein the vector is a plasmid, virus, or Agrobacterium.

15. A plant cell comprising the expression cassette or vector of any one of claims 1 to 14.

16. The plant cell of claim 15, wherein the plant cell is a monocot cell or a dicot cell.

17. The plant cell of claim 16, wherein the monocot plant cell is a Zea mays cell.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A transcription terminator comprising a nucleotide sequence selected from the group comprising:
(a) SEQ ID NOs: 93 to 100; and
(b) a nucleotide sequence having at least 90% sequence identity with one or more of SEQ ID NOs: 93 to 100.

36. An expression cassette comprising:
a promoter operably linked to a transcribable nucleotide sequence; and
the terminator of claim 35 operably linked to the promoter.

37. The expression cassette of claim 36, wherein the terminator enhances expression of the transcribable nucleotide sequence by the promoter.

38. An intron comprising a nucleotide sequence selected from the group comprising:
(a) SEQ ID NOs: 82 to 91; and
(b) a nucleotide sequence having at least 90% sequence identity with one or more of SEQ ID NOs: 82 to 91.

39. (canceled)

40. (canceled)

41. (canceled)

42. (canceled)

43. (canceled)

44. (canceled)

45. (canceled)

46. (canceled)

47. (canceled)

48. A method for increasing the expression of a native gene, comprising the following steps: (a) introducing into at least one cell of a plant the nucleic acid molecule of an enhancer element; (b) introducing of a site-directed nuclease and a repair matrix into at least one cell of a plant, wherein the site-directed nuclease is able to generate at least one double strand break of the DNA in the genome in the promoter, the 5′ UTR, the first intron, the 3′ UTR, or the terminator, or any other part of the gene or surrounding gene regulatory sequence of the native gene of the at least one cell and the repair matrix comprises the nucleic acid molecule of an enhancer element or a fragment thereof; (c) cultivation of the at least one cell of (b) under conditions that allow a homology-directed repair or a homologous recombination, wherein the nucleic acid molecule is integrated from the repair matrix into the genome of the plant in the promoter, the 5′ UTR, the first intron, the 3′ UTR, or the terminator, or any other part of the gene or surrounding gene regulatory sequence of the native gene; and (d) obtaining the plant having increased expression of the native gene from the at least one cell.

49. (canceled)

50. (canceled)
